SEQUENCE LISTING (85 sequences)

SEQ ID NO: 1 - protein, 439 aa, Mus musculus
QAYLQQSGAELVRSGASVKMSCKASGYTFTSYNMHWVKQTPGQGLEWIGYIYPGNGDINYNQKFKGKATLTADTSSNTAYMQISSLTSEDSAVYFCARGGVFDYWGQGTTLIVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 115 aa, Mus musculus
QAYLQQSGAELVRSGASVKMSCKASGYTFTSYNMHWVKQTPGQGLEWIGYIYPGNGDINYNQKFKGKATLTADTSSNTAYMQISSLTSEDSAVYFCARGGVFDYWGQGTTLIVSS

SEQ ID NO: 3 - protein, 25 aa, Mus musculus
QAYLQQSGAELVRSGASVKMSCKAS

SEQ ID NO: 4 - protein, 8 aa, Mus musculus
GYTFTSYN

SEQ ID NO: 5 - protein, 17 aa, Mus musculus
MHWVKQTPGQGLEWIGY

SEQ ID NO: 6 - protein, 8 aa, Mus musculus
IYPGNGDI

SEQ ID NO: 7 - protein, 38 aa, Mus musculus
NYNQKFKGKATLTADTSSNTAYMQISSLTSEDSAVYFC

SEQ ID NO: 8 - protein, 8 aa, Mus musculus
ARGGVFDY

SEQ ID NO: 9 - protein, 11 aa, Mus musculus
WGQGTTLIVSS

SEQ ID NO: 10 - protein, 324 aa, Mus musculus
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - protein, 213 aa, Mus musculus
QIVLTQSPAIVSASPGEKVTITCSGSSSVNYMHWFQQKPGTSPKLWIYSTSNLASGVPGRFSGSGSGTSYSLTISRMEAEDAATYYCQQRTSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 106 aa, Mus musculus
QIVLTQSPAIVSASPGEKVTITCSGSSSVNYMHWFQQKPGTSPKLWIYSTSNLASGVPGRFSGSGSGTSYSLTISRMEAEDAATYYCQQRTSYPYTFGGGTKLEIK

SEQ ID NO: 13 - protein, 23 aa, Mus musculus
QIVLTQSPAIVSASPGEKVTITC

SEQ ID NO: 14 - protein, 5 aa, Mus musculus
SSVNY

SEQ ID NO: 15 - protein, 17 aa, Mus musculus
MHWFQQKPGTSPKLWIY

SEQ ID NO: 16 - skipped

SEQ ID NO: 17 - protein, 36 aa, Mus musculus
NLASGVPGRFSGSGSGTSYSLTISRMEAEDAATYYC

SEQ ID NO: 18 - protein, 9 aa, Mus musculus
QQRTSYPYT

SEQ ID NO: 19 - protein, 10 aa, Mus musculus
FGGGTKLEIK

SEQ ID NO: 20 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - DNA, 1317 bp, Mus musculus
  source 1..1317: mol_type = other DNA
caggcttatctacagcagtctggggctgagctggtgaggtctggggcctcagtgaagatgtcctgcaaggcttctggctacacatttaccagttacaatatgcactgggtaaagcagacacctggacagggcctggaatggattggatatatttatcctggaaatggtgatattaattacaatcagaagtttaagggcaaggccacattgactgcagacacatcctccaacacagcctacatgcagatcagcagcctgacatctgaagactctgcggtctatttctgtgcaagagggggggtctttgactactggggccaaggcaccactctcatagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 22 - DNA, 345 bp, Mus musculus
  source 1..345: mol_type = other DNA
caggcttatctacagcagtctggggctgagctggtgaggtctggggcctcagtgaagatgtcctgcaaggcttctggctacacatttaccagttacaatatgcactgggtaaagcagacacctggacagggcctggaatggattggatatatttatcctggaaatggtgatattaattacaatcagaagtttaagggcaaggccacattgactgcagacacatcctccaacacagcctacatgcagatcagcagcctgacatctgaagactctgcggtctatttctgtgcaagagggggggtctttgactactggggccaaggcaccactctcatagtctcctca

SEQ ID NO: 23 - DNA, 75 bp, Mus musculus
  source 1..75: mol_type = other DNA
caggcttatctacagcagtctggggctgagctggtgaggtctggggcctcagtgaagatgtcctgcaaggcttct

SEQ ID NO: 24 - DNA, 24 bp, Mus musculus
  source 1..24: mol_type = other DNA
ggctacacatttaccagttacaat

SEQ ID NO: 25 - DNA, 51 bp, Mus musculus
  source 1..51: mol_type = other DNA
atgcactgggtaaagcagacacctggacagggcctggaatggattggatat

SEQ ID NO: 26 - DNA, 24 bp, Mus musculus
  source 1..24: mol_type = other DNA
atttatcctggaaatggtgatatt

SEQ ID NO: 27 - DNA, 114 bp, Mus musculus
  source 1..114: mol_type = other DNA
aattacaatcagaagtttaagggcaaggccacattgactgcagacacatcctccaacacagcctacatgcagatcagcagcctgacatctgaagactctgcggtctatttctgt

SEQ ID NO: 28 - DNA, 24 bp, Mus musculus
  source 1..24: mol_type = other DNA
gcaagagggggggtctttgactac

SEQ ID NO: 29 - DNA, 33 bp, Mus musculus
  source 1..33: mol_type = other DNA
tggggccaaggcaccactctcatagtctcctca

SEQ ID NO: 30 - DNA, 972 bp, Mus musculus
  source 1..972: mol_type = other DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 31 - DNA, 639 bp, Mus musculus
  source 1..639: mol_type = other DNA
caaattgttctcacccagtctccagcaatcgtgtctgcatctccaggggagaaggtcaccataacctgcagtggcagctcaagtgtaaattacatgcactggttccagcagaagccaggcacttctcccaaactctggatttatagcacatccaacctggcttctggagtccctggtcgcttcagtggcagtggatctgggacctcttactctctcacaatcagccgaatggaggctgaagatgctgccacttattactgccaacaaaggactagttacccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 32 - DNA, 318 bp, Mus musculus
  source 1..318: mol_type = other DNA
caaattgttctcacccagtctccagcaatcgtgtctgcatctccaggggagaaggtcaccataacctgcagtggcagctcaagtgtaaattacatgcactggttccagcagaagccaggcacttctcccaaactctggatttatagcacatccaacctggcttctggagtccctggtcgcttcagtggcagtggatctgggacctcttactctctcacaatcagccgaatggaggctgaagatgctgccacttattactgccaacaaaggactagttacccgtacacgttcggaggggggaccaagctggaaataaaa

SEQ ID NO: 33 - DNA, 78 bp, Mus musculus
  source 1..78: mol_type = other DNA
caaattgttctcacccagtctccagcaatcgtgtctgcatctccaggggagaaggtcaccataacctgcagtggcagc

SEQ ID NO: 34 - DNA, 15 bp, Mus musculus
  source 1..15: mol_type = other DNA
tcaagtgtaaattac

SEQ ID NO: 35 - DNA, 51 bp, Mus musculus
  source 1..51: mol_type = other DNA
atgcactggttccagcagaagccaggcacttctcccaaactctggatttat

SEQ ID NO: 36 - skipped

SEQ ID NO: 37 - DNA, 108 bp, Mus musculus
  source 1..108: mol_type = other DNA
aacctggcttctggagtccctggtcgcttcagtggcagtggatctgggacctcttactctctcacaatcagccgaatggaggctgaagatgctgccacttattactgc

SEQ ID NO: 38 - DNA, 27 bp, Mus musculus
  source 1..27: mol_type = other DNA
caacaaaggactagttacccgtacacg

SEQ ID NO: 39 - DNA, 30 bp, Mus musculus
  source 1..30: mol_type = other DNA
ttcggaggggggaccaagctggaaataaaa

SEQ ID NO: 40 - DNA, 321 bp, Mus musculus
  source 1..321: mol_type = other DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 41 - protein, 151 aa, Escherichia coli
NSSASSAQVTGTLLGTGKTNTTQMPALYTWQHQIYNVNFIPSSSGTLTCQAGTILVWKNGRETQYALECRVSIHHSSGSINESQWGQQSQVGFGTACGNKKCRFTGFEISLRIPPNAQTYPLSSGDLKGSFSLTNKEVNWSASIYVPAIAK

SEQ ID NO: 42 - protein, 150 aa, Escherichia coli
NLTPQISGTVGDTIQLGTVAPSGAGREIPFALKASSNVGGCASLSTKTADITWSGQLTEKGFANQGGVANDSYVALKTVNGKTQGQEVKASNSTVSFDASKATTDGFKFTAQLKGGQTPGDFQGAAAYAVTYKGGGGGGQQGDVKFFGNV

SEQ ID NO: 43 - protein, 262 aa, Escherichia coli
WMTGDFNGSVDIGGSITADDYRQKWEWKVGTGLNGFGNVLNDLTNGGTKLTITVTGNKPILLGRTKEAFATPVTGGVDGIPHIAFTDYEGASVVLRNPDGETNKKGLAYFVLPMKNAEGTKVGSVKVNASYAGVLGRGGVTSADGELLSLFADGLSSIFYGGLPRGSELSAGSAAAARTKLFGSLSRNDILGQIQRVNANITSLVDVAGSYRENMEYTDGTVVSAAYALGIANGQTIEATFNQAVTTSTQWSAPLNVAITYY

SEQ ID NO: 44 - DNA, 57 bp, Mus musculus
  source 1..57: mol_type = other DNA
atgggattcagcaggatctttctcttcctcctgtcaataactacaggtgtccactcc

SEQ ID NO: 45 - protein, 19 aa, Mus musculus
MGFSRIFLFLLSITTGVHS

SEQ ID NO: 46 - DNA, 66 bp, Mus musculus
  source 1..66: mol_type = other DNA
atgcattttcaagtgcagattttcagcttcctgctaatcagtgcctcagtcatcatgtcccgagga

SEQ ID NO: 47 - protein, 22 aa, Mus musculus
MHFQVQIFSFLLISASVIMSRG

SEQ ID NO: 48 - DNA, 57 bp, Sus scrofa
  source 1..57: mol_type = other DNA
atggagtttggcctgtcctgggtgttcctggtggccctgtttcgcggagtgcagtgc

SEQ ID NO: 49 - protein, 19 aa, Sus scrofa
MEFGLSWVFLVALFRGVQC

SEQ ID NO: 50 - DNA, 66 bp, Sus scrofa
  source 1..66: mol_type = other DNA
atgaagtacctgctgcccacagctgctgctggactgctgctgctggctgctcagcctgctatggcc

SEQ ID NO: 51 - protein, 22 aa, Sus scrofa
MKYLLPTAAAGLLLLAAQPAMA

SEQ ID NO: 52 - protein, 14 aa, Sus scrofa
LLCQEPTDVIIIHS

SEQ ID NO: 53 - protein, 16 aa, Sus scrofa
FQSNETAQNGVLIRIW

SEQ ID NO: 54 - protein, 14 aa, Sus scrofa
ALNVTGPILNFFAN

SEQ ID NO: 55 - protein, 17 aa, Sus scrofa
PQSCSISNKERVVTVIA

SEQ ID NO: 56 - protein, 22 aa, Sus scrofa
LPDTVRAIMDRWTLQMGFPVIT

SEQ ID NO: 57 - protein, 14 aa, Sus scrofa
QHQLQTNLSVIPVI

SEQ ID NO: 58 - protein, 109 aa, Sus scrofa
RADAKPSVFIFPPSKEQLETQTVSVVCLLNSFFPREVNVKWKVDGVVQSSGILDSVTEQDSKDSTYSLSSTLSLPTSQYLSHNLYSCEVTHKTLASPLVKSFNRNECEA

SEQ ID NO: 59 - protein, 328 aa, Sus scrofa
APKTAPSVYPLAPCGRDTSGPNVALGCLASSYFPEPVTMTWNSGALTSGVHTFPSVLQPSGLYSLSSMVTVPASSLSSKSYTCNVNHPATTTKVDKRVGTKTKPPCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTVTCLVIGFYPPDIHVEWKSNGQPEPEGNYRTTPPQQDVDGTFFLYSKLAVDKARWDHGETFECAVMHEALHNHYTQKSISKTQGK

SEQ ID NO: 60 - protein, 328 aa, Sus scrofa
APKTAPSVYPLAPCGRDVSGPNVALGCLASSYFPEPVTVTWNSGALTSGVHTFPSVLQPSGLYSLSSMVTVPASSLSSKSYTCNVNHPATTTKVDKRVGIHQPQTCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTLTCLVIGFYPPDIHVEWKSNGQPEPENTYRTTPPQQDVDGTFFLYSKLAVDKARWDHGDKFECAVMHEALHNHYTQKSISKTQGK

SEQ ID NO: 61 - protein, 328 aa, Sus scrofa
APKTAPSVYPLAPCSRDTSGPNVALGCLASSYFPEPVTVTWNSGALSSGVHTFPSVLQPSGLYSLSSMVTVPASSLSSKSYTCNVNHPATTTKVDKRVGTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 62 - protein, 328 aa, Sus scrofa
APKTAPLVYPLAPCGRDTSGPNVALGCLASSYFPEPVTVTWNSGALTSGVHTFPSVLQPSGLYSLSSMVTVPASSLSSKSYTCNVNHPATTTKVDKRVGTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 63 - protein, 333 aa, Sus scrofa
AYNTAPSVYPLAPCGRDVSDHNVALGCLVSSYFPEPVTVTWNSGALSRVVHTFPSVLQPSGLYSLSSMVIVAASSLSTLSYTCNVYHPATNTKVDKRVDIEPPTPICPEICSCPAAEVLGAPSVFLFPPKPKDILMISRTPKVTCVVVDVSQEEAEVQFSWYVDGVQLYTAQTRPMEEQFNSTYRVVSVLPIQHQDWLKGKEFKCKVNNKDLLSPITRTISKATGPSRVPQVYTLPPAWEELSKSKVSITCLVTGFYPPDIDVEWQSNGQQEPEGNYRTTPPQQDVDGTYFLYSKLAVDKVRWQRGDLFQCAVMHEALHNHYTQKSISKTQGK

SEQ ID NO: 64 - protein, 277 aa, Sus scrofa
TFPSVLQPSGLYSLSSMVTVPASSLSSKSYTCNVNHPATTTKVDKRVGTKTKPPCPICPACEGPGPSAFIFPPKPKDTLMISRTPKVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPPTEELSRSKVTLTCLVTGFYPPDIDVEWQRNGQPEPEGNYRTTPPQQDVDGTYFLYSKLAVDKASWQRGDTFQCAVMHEALHNHYTQKSIFKTPGK

SEQ ID NO: 65 - protein, 318 aa, Sus scrofa
APKTAPSVYPLAPCGRDVSGPNVALGCLASSYFPEPVTVTWNSGALTSGVHTFPSVLQPSGLYSLSSMVTVPASSLSSKSYTCNVNHPATTTKVDKRVGIHQPQTCPICPACEGPGPSAFIFPPKPKDTLMISRTPKVTCVVVDVSQENPEVQFSWYVDGVEVHTAQTRPKEEQFNSTYRVVSVLLIQHQDWLNGKEFKCKVNNKDLPAPITRIISKAKGQTREPQVYTLPPPTEELSRSKVTLTCLVTGFYPPDIDVEWQRNGQPEPEGNYRTTPPQQDVDGTYFLYSKLAVDKASWQRGDTFQCAVMHEALHNHYT

SEQ ID NO: 66 - protein, 324 aa, Sus scrofa
APKTAPSVYPLAPCSRDTSGPNVALGCLVSSYFPEPVTVTWNSGALTSGVHTFPSVLQPSGLYSLSSMVTVPAHSLSSKRYTCNVNHPATKTKVDLCVGRPCPICPGCEVAGPSVFIFPPKPKDILMISRTPEVTCVVVDVSKEHAEVQFSWYVDGEEVHTAETRPKEEQFNSTYRVVSVLPIQHEDWLKGKEFECKVNNEDLPGPITRTISKAKGVVRSPEVYTLPPPAEELSKSIVTLTCLVKSIFPXFIHVEWKINGKPEPENAYRTTPPQEDEDRTYFLYSKLAVDKARWDHGETFECAVMHEALHNHYTQKSISKTQGK

SEQ ID NO: 67 - protein, 317 aa, Sus scrofa
AYNTAPSVYPLAPCGRDVSDHNVALGCLVSSYFPEPVTVTWNWGAQTSGVHTFPSVLQPSGLYSLSSTVTVPAHSLSSKCFTCNVNHPATTTKVDLCVGKKTKPRCPICPGCEVAGPSVFIFPPKPKDILMISRTPEVTCVVVDVSKEHAEVQFSWYVDGEEVHTAETRPKEEQFNSTYRVVSVLPIQHEDWLKGKEFECKVNNEDLPGPITRTISKAKGVVRSPEVYTLPPPAEELSKSIVTLTCLVKSFFPPFIHVEWKINGKPEPENAYRTTPPQEDEDGTYFLYSKFSVEKFRWHSGGIHCAVMHEALHNHYT

SEQ ID NO: 68 - protein, 314 aa, Sus scrofa
APKTAPSVYPLAPCGRDTSGPNVALGCLASSYFPEPVTLTWNSGALTSGVHTFPSVLQPSGLYSLSSMVTVPASSLSSKSYTCNVNHPATTTKVDLCVGRPCPICPACEGPGPSVFIFPPKPKDTLMISRTPQVTCVVVDVSQENPEVQFSWYVDGVEVHTAQTRPKEAQFNSTYRVVSVLPIQHEDWLKGKEFECKVNNKDLPAPITRIISKAKGPSREPQVYTLSPSAEELSRSKVSITCLVTGFYPPDIDVEWKSNGQPEPEGNYRTTPPQQDVDGTYFLYSKLAVDKASWQRGDPFQCAVMHEALHNHYT

SEQ ID NO: 69 - protein, 320 aa, Sus scrofa
APKTAPSVYPLAPCGRDTSGPNVALGCLASSYFPEPVTVTWNSGALTSGVHTFPSVLQPSGLYSLSSTVTVPARSSSRKCFTCNVNHPATTTKVDLCVGRPCPICPACEGNGPSVFIFPPKPKDTLMISRTPEVTCVVVDVSQENPEVQFSWYVDGEEVHTAETRPKEEQFNSTYRVVSVLPIQHQDWLKGKEFECKVNNKDLPAPITRIISKAKGPSREPQVYTLSPSAEELSRSKVSITCLVTGFYPPDIDVEWKSNGQPEPEGNYRSTPPQEDEDGTYFLYSKLAVDKARLQSGGIHCAVMHEALHNHYTQKSISKT

SEQ ID NO: 70 - protein, 341 aa, Sus scrofa
SETSPKIFPLTLGSSEPAGYVVIACLVRDFFPSEPLTVTWSPSREGVIVRNFPPAQAGGLYTMSSQLTLPVEQCPADQILKCQVQHLSKSSQSVNVPCKVLPSDPCPQCCKPSLSLQPPALADLLLGSNASLTCTLSGLKKSEGVSFTWQPSGGKDAVQASPTRDSCGCYSVSSILPGCADPWNKGETFSCTAAHSELKSALTATITKPKVNTFRPQVHLLPPPSEELALNELVTLTCLVRGFSPKDVLVRWLQGGQELPRDKYLVWESLPEPGQAIPTYAVTSVLRVDAEDWKQGDTFSCMVGHEALPLAFTQKTIDRLAGKPTHVNVSVVMAEAEGICY

SEQ ID NO: 71 - DNA, 327 bp, Sus scrofa
  source 1..327: mol_type = other DNA
cgggctgatgccaagccatccgtcttcatcttcccgccatcgaaggagcagttagagacccaaactgtctctgtggtgtgcttgctcaatagcttcttccccagagaagtcaatgtcaagtggaaagtggatggggtggtccaaagcagtggcatcttggatagtgtcacagagcaggacagcaaggacagcacctacagcctcagcagcaccctctcgctgcccacgtcacagtacctaagtcataatttatattcctgtgaggtcacccacaagaccctggcctcccctctggtcaaaagcttcaacaggaacgagtgtgaggct

SEQ ID NO: 72 - DNA, 1079 bp, Sus scrofa
  source 1..1079: mol_type = other DNA
gcccccaagacggccccatcggtctaccctctggccccctgcggcagggacacgtctggccctaacgtggccttgggctgcctggcctcaagctacttccccgagccagtgaccatgacctggaactcgggcgccctgaccagtggcgtgcataccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaacaaagaccaaaccaccatgtcccatatgcccaggctgtgaagtggccgggccctcggtcttcatcttccctccaaaacccaaggacaccctcatgatctcccagacccccgaggtcacgtgcgtggtggtggacgtcagcaaggagcacgccgaggtccagttctcctggtacgtggacggcgtagaggtgcacacggccgagacgagaccaaaggaggagcagttcaacagcacctaccgtgtggtcagcgtcctgcccatccagcaccaggactggctgaaggggaaggagttcaagtgcaaggtcaacaacgtagacctcccagcccccatcacgaggaccatctccaaggctatagggcagagccgggagccgcaggtgtacaccctgcccccacccgccgaggagctgtccaggagcaaagtcaccgtaacctgcctggtcattggcttctacccacctgacatccatgttgagtggaagagcaacggacagccggagccagagggcaattaccgcaccaccccgccccagcaggacgtggacgggaccttcttcctgtacagcaagctcgcggtggacaaggcaagatgggaccatggagaaacatttgagtgtgcggtgatgcacgaggctctgcacaaccactacacccagaagtccatctccaagactcagggtaaatgagccacccgctgcaccccacgtgctctcgggtcccaagagctcgcctaagccccagcgctgtgtacatacgtcccgggccagcatgaaataaa

SEQ ID NO: 73 - DNA, 1079 bp, Sus scrofa
  source 1..1079: mol_type = other DNA
gcccccaagacggccccatcggtctaccctctggccccctgcggcagggacgtgtctggccctaacgtggccttgggctgcctggcctcaagctacttccccgagccagtgaccgtgacctggaactcgggcgccctgaccagtggcgtgcacaccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaatacaccagccgcaaacatgtcccatatgcccaggctgtgaagtggccgggccctcggtcttcatcttccctccaaaacccaaggacaccctcatgatctcccagacccccgaggtcacgtgcgtggtggtggacgtcagcaaggagcacgccgaggtccagttctcctggtacgtggacggcgtagaggtgcacacggccgagacgagaccaaaggaggagcagttcaacagcacctaccgtgtggtcagcgtcctgcccatccagcaccaggactggctgaaggggaaggagttcaagtgcaaggtcaacaacgtagacctcccagcccccatcacgaggaccatctccaaggctatagggcagagccgggagccgcaggtgtacaccctgcccccacccgccgaggagctgtccaggagcaaagtcacgctaacctgcctggtcattggcttctacccacctgacatccatgttgagtggaagagcaacggacagccggagccagagaacacataccgcaccaccccgccccagcaggacgtggacgggaccttcttcctgtacagcaaactcgcggtggacaaggcaagatgggaccatggagacaaatttgagtgtgcggtgatgcacgaggctctgcacaaccactacacccagaagtccatctccaagactcagggtaaatgagccacccgctgcaccccacgtgctctcgggtcccgcgagctcgcctgagccccagcgctgtgtacatacgtcccgggccagcatgaaataaa

SEQ ID NO: 74 - DNA, 1079 bp, Sus scrofa
  source 1..1079: mol_type = other DNA
gcccccaagacggccccatcggtctaccctctggccccctgcagcagggacacgtctggccctaacgtggccttgggctgcctggcctcaagctacttccccgagccagtgaccgtgacctggaactcgggcgccctgtccagtggcgtgcataccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaacaaagaccaaaccaccatgtcccatatgcccagcctgtgaatcaccagggccctcggtcttcatcttccctccaaaacccaaggacaccctcatgatctcccggacaccccaggtcacgtgcgtggtggttgatgtgagccaggagaacccggaggtccagttctcctggtacgtggacggcgtagaggtgcacacggcccagacgaggccaaaggaggagcagttcaacagcacctaccgcgtggtcagcgtcctacccatccagcaccaggactggctgaacgggaaggagttcaagtgcaaggtcaacaacaaagacctcccagcccccatcacaaggatcatctccaaggccaaagggcagacccgggagccgcaggtgtacaccctgcccccacacgccgaggagctgtccaggagcaaagtcagcataacctgcctggtcattggcttctacccacctgacatcgatgtcgagtggcaaagaaacggacagccggagccagagggcaattaccgcaccaccccgccccagcaggacgtggacgggacctacttcctgtacagcaagttctcggtggacaaggccagctggcagggtggaggcatattccagtgtgcggtgatgcacgaggctctgcacaaccactacacccagaagtctatctccaagactccgggtaaatgagccactcgctgcacccctcaagctcttgggtcccaagagctcacctgagccccagccctgtgtacatatcgcccgggccagcatgaaataaa

SEQ ID NO: 75 - DNA, 1079 bp, Sus scrofa
  source 1..1079: mol_type = other DNA
gcccccaagacggccccattggtctaccctctggccccctgcggcagggacacgtctggccctaacgtggccttgggctgcctggcctcaagctacttccccgagccagtgaccgtgacctggaactcgggcgccctgaccagtggcgtgcataccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaacaaagaccaaaccaccatgtcccatatgcccagcctgtgaatcgccagggccctcggtcttcatcttccctccaaaacccaaggacaccctcatgatctcccggacaccccaggtcacgtgcgtggtagttgatgtgagccaggagaacccggaggtccagttctcctggtacgtggacggcgtagaggtgcacacggcccagacgaggccaaaggaggagcagttcaacagcacctaccgcgtggtcagcgtcctgcccatccagcaccaggactggctgaacgggaaggagttcaagtgcaaggtcaacaacaaagacctcccagcccccatcacaaggatcatctccaaggccaaagggcagacccgggagccgcaggtgtacaccctgcccccacacgccgaggagctgtccaggagcaaagtcagcataacctgcctggtcattggcttctacccacctgacatcgatgtcgagtggcaaagaaacggacagccggagccagagggcaattaccgcaccaccccgccccagcaggacgtggacgggacctacttcctgtacagcaagttctcggtggacaaggccagctggcagggtggaggcatattccagtgtgcggtgatgcacgaggctctgcacaaccactacacccagaagtctatctccaagactccgggtaaatgagccactcgctgcacccctcatgctcttgggtcccaagagctcacctgagccccagcgctgtgtacatacgtcccgggccagcatgaaataaa

SEQ ID NO: 76 - DNA, 1517 bp, Sus scrofa
  source 1..1517: mol_type = other DNA
gcctacaacacagctccatcggtctaccctctggccccctgtggcagggacgtgtctgatcataacgtggccttgggctgccttgtctcaagctacttccccgagccagtgaccgtgacctggaactcgggtgccctgtccagagtcgtgcataccttcccatccgtcctgcagccgtcagggctctactccctcagcagcatggtgatcgtggcggccagcagcctgtccaccctgagctacacgtgcaacgtctaccacccggccaccaacaccaaggtggacaagcgtgttggtgagcgcctgcgctgggggcgggagtgcacgtcaagacaggccggggtgagccccctgcccgcagggaccagttcccaggatggagagtcccacccagggtgtccccctcacctgcaggcccaggctcagggaggggtcaactcggcacttcggagaggccagggtgggcacaggcgggacccctcccccggccctgggcccccagcagcacgtgatgctctggcatgtaccaagacctgaccctgacctgagcccagccctgggccctccccacccccagtgaccctgtgtgttctctctgcagacatcgaaccccccacacccatctgtcccgaaatttgctcatgcccaggtgagtcagtcgggcgtgaccctcctcctgaagaggtggcccgagctgggtctcaacacgagggtgcacgggtgcccaggagcaccccaggacaggtgctgaccccacactgtgtctcctccaccagctgcagaggtcctgggagcaccgtcggtcttcctcttccctccaaaacccaaggacatcctcatgatctcccggacacccaaggtcacgtgcgtggtggtggacgtgagccaggaggaggctgaagtccagttctcctggtacgtggacggcgtacagttgtacacggcccagacgaggccaatggaggagcagttcaacagcacctaccgcgtggtcagcgtcctgcccatccagcaccaggactggctgaaggggaaggagttcaagtgcaaggtcaacaacaaagacctcctttcccccatcacgaggaccatctccaaggctacaggtgcatggggtgagaggcagggagggtcccgtggggccacctggggggaccatcatgctaacagaggtgtctggcctcacagggccgagccgggtgccgcaggtgtacaccctgcccccagcctgggaagagctgtccaagagcaaagtcagcataacctgcctggtcactggcttctacccacctgacatcgatgtcgagtggcagagcaacggacaacaagagccagagggcaattaccgcaccaccccgccccagcaggacgtggatgggacctacttcctgtacagcaagctcgcggtggacaaggtcaggtggcagcgtggagacctattccagtgtgcggtgatgcacgaggctctgcacaaccactacacccagaagtccatctccaagactcagggtaaatga

SEQ ID NO: 77 - DNA, 928 bp, Sus scrofa
  source 1..928: mol_type = other DNA
ataccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaacaaagaccaaaccaccatgtcccatatgcccagcctgtgaagggcccgggccctcggccttcatcttccctccaaaacccaaggacaccctcatgatctcccggacccccaaggtcacgtgcgtggtggtagatgtgagccaggagaacccggaggtccagttctcctggtacgtggacggcgtagaggtgcacacggcccagacgaggccaaaggaggagcagttcaacagcacctaccgcgtggtcagcgtcctgcccatccagcaccaggactggctgaacgggaaggagttcaagtgcaaggtcaacaacaaagacctcccagcccccatcacaaggatcatctccaaggccaaagggcagacccgggagccgcaggtgtacaccctgcccccacccaccgaggagctgtccaggagcaaagtcacgctaacctgcctggtcactggcttctacccacctgacatcgatgtcgagtggcaaagaaacggacagccggagccagagggcaattaccgcaccaccccgccccagcaggacgtggacgggacctacttcctgtacagcaagctcgcggtggacaaggccagctggcagcgtggagacacattccagtgtgcggtgatgcacgaggctctgcacaaccactacacccagaagtccatcttcaagactccgggtaaatgagccacttgctgcaccccacgtgctctcgggtcccacgagctcgcctgagccccagccctgtgtacatacgtcccgggccagcatgaaataaa

SEQ ID NO: 78 - DNA, 1489 bp, Sus scrofa
  source 1..1489: mol_type = other DNA
gcccccaagacggccccatcggtctaccctctggccccctgcggcagggacgtgtctggccctaacgtggccttgggctgcctggcctcaagctacttccccgagccagtgaccgtgacctggaactcgggcgccctgaccagtggcgtgcacaccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggtgagcgcctgcgctgggggcgggagtgcacgtcgagacaggccggggtgagccccctgcccgcagggaccagttcccaggatggagagtcccgcccagggtgtcagggcctctcacctctgtccccttcacctgcaggcccaggctcagggaggggtcaactcggcacttcggagaggccagggtgggcacaggcgggacccctcccccggccctgggcccccagcagcacgagctgctctgggatgtaccaagacctgaccctgacctgagcccagccctgggccctccccacccccagtgactctgagtgttctctcctcaggaatacaccagccgcaaacatgtcccatatgcccaggtaagaaactcgggcctcgccctcctccccaaggaggtagcctgagcccaagttcggcccgagggggacaggtgcccaggaggaccccaggccacgagctgacccgcactctgtctcccccaccagcctgtgaagggcccgggccctcggccttcatcttccctccaaaacccaaggacaccctcatgatctcccggacccccaaggtcacgtgcgtggtggttgatgtgagccaggagaacccggaggtccagttctcctggtacgtggacggcgtagaggtgcacacggcccagacgaggccaaaggaggagcagttcaacagcacctaccgcgtggtcagcgtcctgctcatccagcaccaggactggctgaacgggaaggagttcaagtgcaaggtcaacaacaaagacctcccagcccccatcacaaggatcatctccaaggccaaaggtgggcaaggggggcaggcggagagggacctgtggggccacctggggggaccatcatgctaactgaggtgcctggcctcacagggcagacccgggagccgcaggtgtacaccctgcccccacccaccgaggagctgtccaggagcaaagtcacgctaacctgcctggtcactggcttctacccacctgacatcgatgtcgagtggcaaagaaacggacagccggagccagagggcaattaccgcaccaccccgccccagcaggacgtggacgggacctacttcctgtacagcaagctcgcggtggacaaggccagctggcagcgtggagacacattccagtgtgcggtgatgcacgaggctctgcacaaccactacaccc

SEQ ID NO: 79 - DNA, 1485 bp, Sus scrofa
  unsure 1258: note = /n="a or g or c or t"
  source 1..1485: mol_type = other DNA
gcccccaagacggccccatcggtctaccctctggccccctgcagcagggacacgtctggccctaacgtggccttgggctgcctggtctcaagctacttccccgagccagtgaccgtgacctggaactcgggcgccctgaccagtggcgtgcacaccttcccatccgtcctgcagccgtcagggctctactccctcagcagcatggtgaccgtgccggcccacagcttgtccagcaagcgctatacgtgcaatgtcaaccacccagccaccaaaaccaaggtggacctgtgtgttggtgagtgcctgggctgggggcgggtgtgcagacgggctgaggtcagccttcctgccgagagaccaccccaccagggtgtcagggccgctcacctctgtccccttcacctgcaggcccaggctcagggaggggtcaactcggcacttcggagaggccagggtgggcacaggcgggacccctcccccggccctgggcccccagcagcacgagctgctctgggatgtaccaagacctgaccctgacctgagcccagccctggaccctccccacccccagtgactctgtgtgttctctcctcaggacgaccatgtcccatatgcccaggtaagaaactcgggcctcgccctcctccccaaggaggtagcctgagcccaggttcggcccgaggggggacaggtgcccaggaggaccccaggccacgagctgacccgcactctgtctcccccaccaggctgtgaagtggccgggccctcggtcttcatcttccctccaaaacccaaggacatcctcatgatctcccggacccccgaggtcacgtgcgtggtggtggacgtcagcaaggagcacgccgaggtccagttctcctggtacgtggacggcgaagaggtgcacacggccgagacgaggccaaaggaggagcagttcaacagcacctaccgcgtggtcagcgtcctgcccatccagcacgaggactggctgaaggggaaggagttcgagtgcaaggtcaacaacgaagacctcccaggccccatcacgaggaccatctccaaggccaaaggtgggcaaggtgggcaggcggagagggtcccgtgcggccacctggggggaccatcatgctaactgaggtgcctggcttcacaggggtggtacggagcccggaggtgtacaccctgcccccacccgccgaggagctgtccaagagcatagtcacgctaacctgcctggtcaaaagcatcttcccgnctttcatccatgttgagtggaaaatcaacggaaaaccagagccagagaacgcatatcgcaccaccccgcctcaggaggacgaggacaggacctacttcctgtacagcaagctcgcggtggacaaggcaagatgggaccatggagaaacatttgagtgtgcggtgatgcacgaggctctgcacaaccactacacccagaagtccatctccaagactcagggtaaatga

SEQ ID NO: 80 - DNA, 1462 bp, Sus scrofa
  source 1..1462: mol_type = other DNA
gcctacaacacagctccatcggtctaccctctggccccctgtggcagggacgtgtctgatcataacgtggccttgggctgcctggtctcaagctacttccccgagccagtgaccgtgacctggaactggggcgcccagaccagtggcgtgcacaccttcccatccgtcctgcagccgtcagggctctactccctcagcagcacggtgaccgtgccggcccacagcttgtccagcaagtgcttcacgtgcaatgtcaaccacccggccaccaccaccaaggtggacctgtgtgttggtgagtgcctggactgggggcgggtgtgcagacgggctgaggtcagccttcctgccgagagaccacccccccagggtgtcagggccgctcacctctgtccccctcacctgtaggcccaggctcagggaggggtcaactcggcacttcggagaggccagggtgggcacaggcgggacccctcccccggccctgggcccccagcagcacgagctgctctgggatgtaccaagacctgaccctgacctgagcccagccctgggccctccccacccccagtgactctgtgtgttctctcctcaggaaaaaagaccaagcctcgatgtcccatatgcccaggtaagaaactcgggcctcgccctcctccccaaggaggtagcctgagcccaggttcggcccgaggggggacaggtgcccaggaggaccccaggccacgagctgacccgcactctgtctcccccaccaggctgtgaagtggccgggccctcggtcttcatcttccctccaaaacccaaggacatcctcatgatctcccggacccccgaggtcacgtgcgtggtggtggacgtcagcaaggagcacgccgaggtccagttctcctggtacgtggacggcgaagaggtgcacacggccgagacgagaccaaaggaggagcagttcaacagcacttaccgcgtggtcagcgtcctgcccatccagcacgaggactggctgaaggggaaggagttcgagtgcaaggtcaacaacgaagacctcccaggccccatcacgaggaccatctccaaggccaaaggtgggcaaggtgggcaggcggagagggtcccgtgcggccacctggggggaccatcatgctaactgaggtgcctggcttcacaggggtggtacggagcccggaggtgtacaccctgcccccacccgccgaggagctgtccaagagcatagtcacgctaacctgcctggtcaaaagcttcttcccgcctttcatccatgttgagtggaaaatcaacggaaaaccagagccagagaacgcataccgcaccaccccgccccaggaggacgaggacgggacctacttcctgtacagcaagttctcggtggaaaagttcaggtggcacagtggaggcatccactgtgcggtgatgcacgaggctctgcacaaccactacaccc

SEQ ID NO: 81 - DNA, 1583 bp, Sus scrofa
  source 1..1583: mol_type = other DNA
gcccccaagacggccccatcggtctaccctctggccccctgcggcagggacacgtctggccctaacgtggccttgggctgcctggcctcaagctacttccccgagccagtgaccctgacctggaactcgggcgccctgaccagtggcgtgcataccttcccatccgtcctgcagccgtcagggctctactccctcagcagcatggtgaccgtgccggccagcagcctgtccagcaagagctacacctgcaatgtcaaccacccggccaccaccaccaaggtggacctgtgtgttggtgagtgcctgggctgggggcaggtgtgtagacgggctgaggtcagccttcctgccgagagaccaccccaccagggtgtcagggccgctcacctctgtccccctcacctgcaggcccaggctcagggaggggtcaactcggcacttcggagaggccagggtgggcacaggcgggacccctcccccggccctgggcccccagcagcacgagctgctctgggatgtaccaagacctgaccctgacctgagcccaaccctggaccctccctacccccagtgactctgtgtgttctctcctcaggacgaccatgtcccatatgcccaggtaagaaactcgggcttcgccctcctccccaaggaggtagcctgagcccaggttcggcccgaggggggacaggtgcccaggaggaccccaggccacgagctgacccgcactctgtctcccccaccagcctgtgaagggcccgggccctcggtcttcatcttccctccaaaacccaaggacaccctcatgatctcccggacaccccaggtcacgtgcgtggtggtagatgtgagccaggaaaacccggaggtccagttctcctggtatgtggacggtgtagaggtgcacacggcccagacgaggccaaaggaggcgcagttcaacagcacctaccgtgtggtcagcgtcctgcccatccagcacgaggactggctgaaggggaaggagttcgagtgcaaggtcaacaacaaagacctcccagcccccatcacaaggatcatctccaaggccaaaggtgggcaaggggggcaaggggggcaggcggagagggacctgtggggccacctggggggaccatcatgctaacagaggtgcctggcttcacagggccgagccgggagccgcaggtgtacaccctgtccccatccgccgaggagctgtccaggagcaaagtcagcataacctgcctggtcactggcttctacccacctgacatcgatgtcgagtggaagagcaacggacagccggagccagagggcaattaccgcaccaccccgccccagcaggacgtggacgggacctacttcctgtacagcaagctcgcggtggacaaggccagctggcagcgtggagacccattccagtgtgcggtgatgcacgaggctctgcacaaccactacacccagaagtccatcttcaagactccgggtaaatgagccacttgcaccccacgtgctctcgggtcccacgagctcgcctgagccccagccctgtgtacatacgtcccgggccagcatgaaataaa

SEQ ID NO: 82 - DNA, 1479 bp, Sus scrofa
  source 1..1479: mol_type = other DNA
gcccccaagacggccccatcggtctaccctctggccccctgcggcagggacacgtctggccctaacgtggccttgggctgcctggcctcaagctacttccccgagccagtgaccgtgacctggaactcgggcgccctgaccagtggcgtgcacaccttcccatccgtcctgcagccgtcagggctctactccctcagcagcacggtgaccgtgccggccaggagctcgtccagaaagtgcttcacgtgcaatgtcaaccacccggccaccaccaccaaggtggacctgtgtgttggtgagtgcctgggctgggggcaggtgtgtagacgggctgaggtcagccttcctgccgagagaccaccccaccagggtgtcagggccgctcacctctgtccccctcacctgcaggcccaggctcagggaggggtcaactcggcacttcggagaggccagggtgggcacaggcgggacccctcccccggccctgggcccccagcagcacgagctgctctgggatgtaccaagacctgaccctgacctgagcccaaccctggaccctccctacccccagtgactctgtgtgttctctcctcaggacgaccatgtcccatatgcccaggtaagaaactcgggcttcgccctcctccccaaggaggtagcctgagcccaggttcggcccgaggggggacaggtgcccaggaggaccccaggccacgagctgacccgcactctgtctcccccaccagcctgtgaagggaacgggccctcggtcttcatcttccctccaaaacccaaggacaccctcatgatctcccggacccccgaggtcacgtgcgtggtggtagatgtgagccaggaaaacccggaggtccagttctcctggtacgtggacggcgaagaggtgcacacggccgagacgaggccaaaggaggagcagttcaacagcacctaccgtgtggtcagcgtcctgcccatccagcaccaggactggctgaagggaaaggagttcgagtgcaaggtcaacaacaaagacctcccagcccccatcacaaggatcatctccaaggccaaaggtgggcaaggggggcaaggggggcaggcggagagggacctgtggggccacctggggggaccatcatgctaacagaggtgcctggcttcacagggccgagccgggagccgcaggtgtacaccctgtccccatccgccgaggagctgtccaggagcaaagtcagcataacctgcctggtcactggcttctacccacctgacatcgatgtcgagtggaagagcaacggacagccggagccagagggcaattaccgctccaccccgccccaggaggacgaggacgggacctacttcctgtacagcaaactcgcggtggacaaggcgaggttgcagagtggaggcatccactgtgcggtgatgcacgaggctctgcacaaccactacacccagaagtccatctccaagact

SEQ ID NO: 83 - DNA, 1023 bp, Sus scrofa
  source 1..1023: mol_type = other DNA
agcgagacctctccaaagatcttcccactgacactgggatcttccgagccagctggctacgtggtcatcgcctgtctggtgagggacttctttccttccgagccactgaccgtgacatggtcccctagcagggagggcgtgatcgtgcggaattttccaccagctcaggctggaggactgtataccatgagctctcagctgacactgcctgtggagcagtgcccagccgatcagatcctgaagtgtcaggtgcagcatctgagcaagtccagccagtctgtgaatgtgccctgcaaggtgctgccttctgacccctgtcctcagtgctgtaagccttctctgtccctgcagcctccagctctggccgatctgctgctgggcagcaacgcttctctgacctgcacactgagcggcctgaagaagtctgagggcgtgtccttcacctggcagccatctggaggcaaggacgctgtgcaggcttccccaacaagggatagctgcggatgttactccgtgtcctccatcctgccaggatgcgctgacccatggaacaagggagagacattctcctgtaccgctgcccacagcgagctgaagtctgctctgaccgccacaatcaccaagcccaaggtgaacacctttagacctcaggtgcatctgctgccccctccaagcgaggagctggctctgaatgagctggtgacactgacctgtctggtgagaggcttttctcctaaggacgtgctggtgcgctggctgcagggaggacaggagctgccaagggataagtacctggtgtgggagtccctgccagagccaggacaggctatcccaacatatgctgtgaccagcgtgctgagggtggacgctgaggattggaagcagggcgacaccttcagctgcatggtgggacacgaggctctgccactggcctttacacagaagaccatcgataggctggctggcaagcctacacatgtgaacgtgtctgtggtcatggctgaggccgagggcatctgttac

SEQ ID NO: 84 - DNA, 1377 bp, Mus musculus
  source 1..1377: mol_type = other DNA
atgggattcagcaggatctttctcttcctcctgtcaataactacaggtgtccactcccaggcttatctacagcagtctggggctgagctggtgaggtctggggcctcagtgaagatgtcctgcaaggcttctggctacacatttaccagttacaatatgcactgggtaaagcagacacctggacagggcctggaatggattggatatatttatcctggaaatggtgatattaattacaatcagaagtttaagggcaaggccacattgactgcagacacatcctccaacacagcctacatgcagatcagcagcctgacatctgaagactctgcggtctatttctgtgcaagagggggggtctttgactactggggccaaggcaccactctcatagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 85 - DNA, 708 bp, Mus musculus
  source 1..708: mol_type = other DNA
atgcattttcaagtgcagattttcagcttcctgctaatcagtgcctcagtcatcatgtcccgaggacaaattgttctcacccagtctccagcaatcgtgtctgcatctccaggggagaaggtcaccataacctgcagtggcagctcaagtgtaaattacatgcactggttccagcagaagccaggcacttctcccaaactctggatttatagcacatccaacctggcttctggagtccctggtcgcttcagtggcagtggatctgggacctcttactctctcacaatcagccgaatggaggctgaagatgctgccacttattactgccaacaaaggactagttacccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

(19) United States
(12) Patent Application Publication
(10) Pub. No.: US 2024/0392036 A1
(43) Pub. Date: Nov. 28, 2024

(54) AMINOPEPTIDASE N-SPECIFIC MONOCLONAL ANTIBODIES AND USES THEREOF

(71) Applicant: Universiteit Gent, Gent (BE)

(72) Inventors: Bert Devriendt, Mariakerke (BE); Eric Cox, Merelbeke (BE)

(73) Assignee: Universiteit Gent, Gent (BE)

(21) Appl. No.: 18/691,384

(22) PCT Filed: Sep. 13, 2022
(86) PCT No.: PCT/EP22/75455

§ 371 Date: Mar. 12, 2024

(30) Foreign Application Priority Data
Sep. 13, 2021 (EP) 21196439.0

(51) Int. Cl. C07K 16/40; A61K 9/00; A61K 39/00; A61K 47/68; A61K 49/00; A61P 37/04

(52) U.S. Cl. CPC C07K 16/40; A61K 9/0053; A61K 47/6871; A61K 49/0002; A61P 37/04; A61K 2039/505; C07K 2317/622

(57) ABSTRACT

The present invention relates to antibodies and antigen-binding fragments that bind specifically to amino peptidase N, fusion constructs and uses thereof, in particular for the targeted delivery of molecules. More specific, the invention provides methods for vaccination, in particular oral vaccination inducing both mucosal and systemic immune responses and methods for treating, reducing or preventing gastrointestinal diseases using said antibodies or fragments.

### FIELD OF THE INVENTION

The present invention relates to antibodies and antigen-binding fragments that bind specifically to aminopeptidase N, fusion constructs and uses thereof, in particular for the targeted delivery of molecules. More specific, the invention provides methods for vaccination, in particular oral vaccination inducing both mucosal and systemic immune responses and methods for treating, reducing or preventing gastrointestinal diseases using said antibodies or fragments.

### BACKGROUND TO THE INVENTION

Most pathogens invade the host at the mucosal surfaces, such as the gut. Frontline protection against these enteropathogens requires robust intestinal immune responses at the site of infection, more specific pathogen-specific secretory immunoglobulin A (SIgA). In contrast to systemic administration, delivery of vaccines to the intestinal mucosa can elicit protective SIgA responses at both local and distal mucosal sites as well as systemic immunity. Oral vaccines have many advantages, nonetheless, oral vaccination and the induction of robust protective immune responses faces many hurdles. Vaccine antigens not only need to survive the gastric pH and degradation by proteolytic enzymes in the gastrointestinal tract, they also must reach the gut-associated lymphoid tissue. However, the small intestinal epithelial barrier restricts uptake of macromolecules, leading to a poor uptake of vaccines at the intestinal surfaces. In addition, without proper activation and correct dosing, tolerance is induced rather than protective immunity.

To overcome these challenges in oral vaccination, current efforts are focused on different encapsulation strategies to preserve antigen stability in the gut, novel mucosal adjuvants to surmount tolerance or targeting antigens to intestinal cell populations to enhance vaccine uptake. For instance, the glycoprotein-2 (GP2) protein is specifically expressed on the apical side of mature M cells and can recognize the bacterial FimH, a component of type I pili on the bacterial outer membrane. Uptake of FimH+ bacteria by M-cells via GP2 was able to initiate mucosal immune responses in mice. An alternative strategy would be to target vaccine antigens towards enterocytes, since these cells are more abundant than M cells in the small intestinal epithelium. For example, targeting receptors involved in transcytosis such as the neonatal Fc-receptor (FcRn) enabled the uptake of antigen-bound IgG Fc-fragments. Another interesting target is aminopeptidase N (APN; CD13). In enterocytes, this membrane glycoprotein is involved in digestive processes by removing N-terminal amino acids from peptides. APN is also expressed on specific subsets of dendritic cells in humans, pigs and mice, which play a central role in the induction of adaptive immune responses. Previous research identified APN as a receptor for F4 fimbriae and was shown to be involved in the epithelial transcytosis of these fimbriae. Interestingly, oral administration of purified F4 fimbriae to piglets triggered protective SIgA responses (Van den Broeck W. 1999). Moreover, delivery of antigens and/or microparticles to aminopeptidase N by different antibody formats facilitated their uptake by the small intestinal epithelium and elicited strong immune responses in piglets upon oral administration (Cox E. WO2009103555; Melkebeek V. 2012; Baert K. 2015).

The inventors of the present invention have identified specific monoclonal antibody constructs which are able to specifically target a clinically relevant antigen towards APN and may be used in for example oral vaccination strategies. The unique data in the present invention demonstrate that after oral delivery the polypeptide not only binds APN, but also leads to endocytosis (transport into the cell) by the intestinal enterocytes and subsequent transcytosis (transport across the interior of the cell) through the epithelial barrier with appropriate presentation to the mucosal immune system. In an example, the inventors demonstrate that fusion constructs linked with the FedF tip adhesin from F18 fimbriated E. coli, which is a clinically relevant but low immunogenic antigen triggers immune responses in piglets upon oral administration. Based on the obtained amino acid data, an isolated antibody, or antigen-binding fragment thereof or chimeric antibody is proposed as well as a chimeric molecule comprising the antibody and an antigen.

Furthermore, the invention provides a method for vaccination, in particular oral vaccination inducing both mucosal and systemic immune responses; and a method for treating, reducing or preventing for example gastrointestinal diseases using said antibodies or fragments.

### SUMMARY OF THE INVENTION

The current invention provides a polypeptide capable of binding APN, inducing transport across the intestinal epithelium after oral delivery and inducing an immune response, in particular against a linked compound.

In a first aspect, the present invention relates to an isolated antibody, antigen-binding fragment thereof or chimeric antibody, that binds aminopeptidase N, wherein said antibody comprises: three heavy chain complementarity determining regions (CDRs) (HCDR1, HCDR2 and HCDR3) as set forth in resp. amino acid sequences SEQ ID NOs: 4, 6 and 8, and optionally one, two or three light chain complementarity determining regions (LCDR1, LCDR2 and LCDR3) as set forth in amino acid sequences resp. SEQ ID NOs: 14, 16 and 18.

In a particular aspect, the present invention relates to an isolated antibody, antigen-binding fragment thereof or chimeric antibody, that binds aminopeptidase N, wherein said antibody comprises: three heavy chain complementarity determining regions (CDRs) (HCDR1, HCDR2 and HCDR3) as set forth in resp. amino acid sequences SEQ ID NOs: 4, 6 and 8, and three light chain complementarity determining regions (LCDR1, LCDR2 and LCDR3) as set forth in amino acid sequences resp. SEQ ID NOs: 14, 16 and 18.

In a specific embodiment of the present invention, the heavy chain variable region (HCVR) has at least 80% amino acid sequence identity to SEQ ID NO: 2; and optionally a light chain variable region (LCVR) having at least 80% amino acid sequence identity to SEQ ID NO: 12.

In another specific embodiment of the present invention, the antibody comprises a HCVR as set forth in SEQ ID NO: 2 and optionally a LCVR as set forth in SEQ ID NO: 12, in particular a heavy chain as set forth in SEQ ID NO: 1 and optionally a light chain as set forth in SEQ ID NO: 11.

In yet a further embodiment, the antibody comprises a HCVR as set forth in SEQ ID NO: 2, an LCVR as set forth in SEQ ID NO: 12, a heavy chain constant region (HCCR) as set forth in SEQ ID NOs: 59-70 and a light chain constant region (LCCR) as set forth in SEQ ID NO: 58.

In a specific embodiment of the present invention, the antibody is a monoclonal antibody, a purified antibody, a recombinant antibody, single domain antibody (also referred to as nanobody or VHH antibody), Fab, Fab′, F(ab′)2, Fv or scFv.

In yet another embodiment, the present invention provides an antibody or antigen-binding fragment thereof, wherein the antibody specifically binds an epitope in the aminopeptidase N protein; and wherein said epitope comprises at least one of the following amino acids sequence SEQ ID NO: 52, 53, 54, 55, 56, or 57 or a sequence having at least 92% identity hereto.

In a further aspect, the present invention provides a chimeric molecule comprising the antibody or antigen-binding fragment thereof and a compound, in particular a bio-active compound, more in particular an antigen.

In another specific embodiment, the present invention provides a chimeric molecule, or an antibody or antigen-binding fragment thereof; wherein said chimeric molecule or antibody or antigen-binding fragment thereof binds aminopeptidase N with a binding affinity between about 10⁻¹² to 10⁻⁸ M, about 10⁻¹¹ to 10⁻⁸ M, more specifically between about 10⁻¹ to 10⁻⁹ M, even more specifically with a binding affinity of about 4×10⁻⁹ M or less, about 3×10⁻⁹ M or less, about 2×10⁻⁹ M or less, preferably about 4×10^{˜9} M or less. In another embodiment, the present invention provides an isolated nucleic acid encoding the antibody, an antigen-binding fragment thereof or chimeric antibody; or a chimeric molecule; wherein said nucleic acids preferably comprises a nucleic acid sequence that has at least 95% identity to the HCVR as set forth in SEQ ID NO: 22 and optionally comprising a nucleic acid sequence that has at least 95% identity to the LCVR as set forth in SEQ ID NO: 32.

In yet a further embodiment, the present invention provides a vector comprising said isolated nucleic acid.

In another particular embodiment, the present invention provides a host cell expressing the antibody, or an antigen-binding fragment thereof; or comprising said nucleic acid or said vector.

In a further aspect, the present invention also provides a pharmaceutical composition comprising said antibody or an antigen-binding fragment thereof; or said chimeric molecule; or said nucleic acid; or said vector; or said host cell; and a pharmaceutically acceptable diluent and/or carrier, and optionally a further therapeutic agent.

In another embodiment, the present invention provides said antibody or an antigen-binding fragment thereof; or said chimeric molecule; or said nucleic acid; or said vector; or said host cell; or said pharmaceutical composition, for use in human or veterinary medicine.

In a specific embodiment, the present invention provides said antibody or an antigen-binding fragment thereof; or said chimeric molecule; or said nucleic acid; or said vector; or said host cell; or said pharmaceutical composition, for use in vaccination; in particular mucosal vaccination; alternatively for use in treating or preventing an intestinal disease, in particular a gut or intestinal infection or inflammation.

In yet a further aspect, the present invention relates to a method for making said antibody or antigen-binding fragment, comprising: (a) introducing into a host cell one or more polynucleotides encoding said antibody or antigen-binding fragment; (b) culturing the host cell under conditions favorable to expression of the one or more polynucleotides; and (c) optionally, isolating the antibody or antigen-binding fragment from the host cell and/or a medium in which the host cell is grown.

In another embodiment, said antibody or antigen-binding fragment is obtainable by said method.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Screening of APN-specific monoclonal antibodies. Binding to porcine aminopeptidase N (APN) was analyzed using (a) enzyme-linked immunosorbent assay (ELISA) with purified kidney APN and (b) flow cytometry using an APN-expressing cell line. O.D.: Optical density; MFI: Mean fluorescence intensity. O.D. values are subtracted from mean background absorbance. MFI values are subtracted from relevant isotype controls. (c) Binding kinetics of several mAbs using bio-layer interferometry (BLI) with resulting affinity (Kn) values. Shift in wavelength (nm) is given over time (s). (d) Resultant heat map of epitope binning assay using BLI. Grey indicates ability of secondary Ab (top row) to bind to APN after primary Ab binding (left column). Black indicates inability of secondary Ab to further bind APN.

FIG. 2: Immune responses after oral administration of APN-specific mouse-IgG2a antibody constructs. FedF-specific IgG (Left) and IgA (Right) serum titers at 0, 9, 14, 21 and 28 dppi (days post primary immunization). Arrows indicate days of oral immunization. n=3.

FIG. 3: Binding kinetics, production and uptake of IMM013-derived constructs. (a) Binding kinetics for α-APN-mIgG1 (clone IMM013) and FedF-linked α-APN-mIgG1 using bio-layer interferometry (BLI) with corresponding affinity values (KD). Shift in wavelength (nm) is given over time (s). (b) Production (O.D.) and binding (MFI) analysis of α-APN-pIgA-FedF and its mutant control construct pIgA-FedF by a porcine IgA-specific ELISA and flow cytometry (FCM) using BHK-APN cells respectively. Medium represents the BHK-APN cell culture medium. O.D.: Optical density; MFI: Mean fluorescence intensity.

FIG. 4: Experimental overview. (a) Timeline of oral immunization experiment with serum and PBMC collection days and oral immunization time points. (b) Overview of different antibody constructs. mIgG1: mouse IgG1; pIgA: pig IgA.

FIG. 5: Increased serum responses after oral immunization with APN-specific antibody constructs. (a) FedF-specific and (b) IMM013-specific IgG and IgA serum titers 0, 9, 14, 21 and 28 dppi (days post primary immunization). OD: optical density. Arrows indicate days of immunization. Multiplicity adjusted p-values: *: p<0.05; **: p<0.01; ***: p<0.001; ****: p<0.0001; * indicates significant differences compared to mIgG1 isotype ctrl or pIgA-FedF ctrl; A indicates significant differences compared to pIgA-FedF ctrl.

FIG. 6: Antigen-specific antibody secreting cells after oral immunization with APN-specific antibody constructs. ELISpot of (a) IgG1- and (b) FedF-specific IgA ASCs from PBMCs isolated on 0, 9, 14, 21 and 28 dppi (days post primary immunization) and mononuclear cells isolated from mesenteric lymph nodes and intestinal tissues 28 dppi. Arrows indicate days of immunization. Multiplicity adjusted p-values: *: p<0.05; **: p<0.01; ***: p<0.001; ****: p<0.0001; * indicates significant differences compared to migG1 isotype or pIgA-FedF ctrl on same day, while A indicates significant differences for each group compared to day 0. migG1: mouse IgG1; pIgA: pig IgA; MLN: mesenteric lymph nodes; JJLP: jejunal lamina propria; JJPP: Jejunal Peyer's Patches; ILP: Ileal lamina propria; IPP: Ileal Peyer's Patches.

FIG. 7: Nucleic acid sequence anti-APN monoclonal antibodies heavy and light chain. Abbreviation: CDR=complementarity determining regions; FR=framework region.

FIG. 8: Decrease in bacterial shedding after oral immunization with an APN-specific antibody-antigen fusion construct. a) Timeline of oral challenge experiment with different oral immunization time points, day of challenge infection and fecal collection time points. b) Mean fecal excretion of F18-fimbriated Shiga-toxin producing E. coli (STEC) per gram of feces (CFU/g). CFU: Colony forming units. *indicates significant differences compared to the PBS control group at each specific day. *: p<0.05; t: One animal was euthanized in the PBS control group due to severe symptoms of edema disease. n=8.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements, or method steps. The terms also encompass “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those skilled in the art. For example, in the following claims, any of the claimed embodiments can be used in any combination.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention. All documents cited in the present specification are hereby incorporated by reference in their entirety.

The present invention relates to antibodies and antigen-binding fragments that bind specifically to amino peptidase N, fusion constructs and uses thereof, in particular for the targeted delivery of molecules. More specific, the invention provides methods for vaccination, in particular oral vaccination inducing both mucosal and systemic immune responses and methods for treating, reducing or preventing gastrointestinal diseases using said antibodies or fragments.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As defined herein, the term “polypeptide” is in particular used in the context of an antibody or antigen-binding fragment thereof. Thus, when the term polypeptide is used, it is meant to be an antibody or antigen-binding fragment thereof.

In one embodiment, the present invention provides a polypeptide, in particular an antibody or antigen-binding fragment thereof, specifically binding APN and furthermore showing specific uptake by cells via the APN receptor, in particular by APN expressing cells, more in particular by enterocytes. As used herein, “enterocytes”, or intestinal absorptive cells (also referred to as absorptive villus epithelial cells), are epithelial cells which line the inner surface of the small and large intestines and possess phagocytosis and transcytosis capacities to transport enteric pathogens or macromolecules across the epithelial barrier. “Aminopeptidase N (APN)” (also known as CD13, ANPEP, PEPN, alanyl aminopeptidase) is a type II membrane glycoprotein, which belongs to the family of membrane-bound metalloproteases and is expressed in a variety of mammalian tissues among which the intestinal brush border membranes.

Relevant structural information for APN may be found, for example, at UniProt or GenBank Accession Numbers as depicted in the Table 1 below.

TABLE 1

| Protein | | |
|---|---|---|
| Accession | | |
| No. | cDNA Accession No. | Organism |
| P15145.4 | NM_214277 (version: NM_214277.1) | Sus scrofa |
| P15144.4 | NM_001150 (version: NM_001150.3) | Homo sapiens |
| P79143.2 | NM_001146034 | Canis lupus |
| | (version: NM_001146034.1) | familiaris |
| P79171.3 | NM_001009252 | Felis catus |
| | (version: NM_001009252.2) | |
| | GDHK01043038.1 | Equus caballus |
| P79098.4 | NM_001075144 | Bos Taurus |
| | (version: NM_001075144.1) | |

As used herein the “APN” or “CD13” polypeptide is meant to be a protein encoded by a mammalian APN gene, including allelic variants as well as biologically active fragments thereof containing conservative or non-conservative changes as well as artificial proteins that are substantially identical, i.e. at least 70%, 75%, 80%, 85%, 87%, 89%, 90%, 92%, 93%, 94%, 95%, 96%, 97%, 98%, or 99% identical to any one of the aforementioned APN polypeptides. In a particular embodiment the APN polypeptide is at least 70%, 75%, 80%, 85%, 87%, 89%, 90%, 92%, 93%, 94%, 95%, 96%, 97%, 98%, or 99% identical to the porcine or pig APN (Sus scrofa; Accession No. ADX53333.1). In a further embodiment, the APN polypeptides as defined herein are further characterized in that they are glycosylated.

By analogy, the “APN” or “CD13” polynucleotide is meant to include allelic variants as well as biologically active fragments thereof containing conservative or non-conservative changes as well as any nucleic acid molecule that is substantially identical, i.e. at least 70%, 75%, 80%, 85%, 87%, 89%, 90%, 92%, 93%, 94%, 95%, 96%, 97%, 98%, or 99% identical to any one of the aforementioned APN encoding polynucleotides.

In a particular embodiment the APN polynucleotide is at least 70%, 75%, 80%, 85%, 87%, 89%, 90%, 92%, 93%, 94%, 95%, 96%, 97%, 98%, or 99% identical to the nucleic acid molecule encoding for porcine APN (Genbank Accession No HQ824547.1).

In one embodiment, the polypeptide of the invention specifically binds or reacts to and is internalized by mammalian intestinal APN, in particular porcine APN, more in particular porcine intestinal APN, more in particular expressed on epithelial cells or enterocytes present in the small intestine, said APN comprising the amino acid sequence represented by protein accession No. ADX53333.1. The unique data in the present invention demonstrate that after oral delivery the polypeptide not only binds APN, but also leads to endocytosis (transport into the cell) by the intestinal enterocytes and subsequent transcytosis (transport across the interior of the cell) through the epithelial barrier with appropriate presentation to the mucosal immune system.

APN expressing cells can be used to determine uptake or internalization of the polypeptide of the invention. The “expression” generally refers to the process by which polynucleotides are transcribed into mRNA and/or the process by which the mRNA is subsequently translated into peptides, polypeptides or proteins. APN expression may be facilitated or increased by methods that involve the introduction of exogenous nucleic acid into the cell. Such a cell may comprise a polynucleotide or vector in a manner that permits expression of an encoded APN polypeptide. Polynucleotides that encode APN as provided herein may be introduced into the host cell as part of a circular plasmid, or as linear DNA comprising an isolated protein-coding region, or in a viral vector. Methods for introducing exogenous nucleic acid into the host cell well known and routinely practiced in the art include transformation, transfection, electroporation, nuclear injection, or fusion with carriers such as liposomes, micelles, ghost cells, and protoplasts. Host cell systems of the invention include plant, invertebrate and vertebrate cells systems.

Cells to be used for studying the internalization are primary cells isolated from small intestinal epithelial tissue, such as for example enterocyte preparations; or continuous cells including recombinant cell lines expressing APN, in particular porcine intestinal epithelial cells (IPEC-J2, IPEC-I, IPI-2i), baby hamster kidney (BHK) cells (e.g. BHK21 cells), or “mini-intestines” derived either from adult ISCs (enteroids/organoids) or from induced pluripotent stem cells (iPSCs)(organoids). Further cells may include, but are not limited to, the following: insect cells, porcine kidney (PK) cells, porcine kidney cortex (SK-RST) cells, feline kidney (FK) cells, felis catus whole foetus cells (Fcwf-4), swine testicular (ST) cells, African green monkey kidney cells (MA-104, MARC-145, VERO, and COS cells), Chinese hamster ovary (CHO) cells, human 293 cells, and murine 3T3 fibroblasts, human colon carcinoma epithelial (CaCo2) cells, human lymphoblast (Kasumi-3), human myeloblast (Kasumi-4), human myeloblast (Kasumi-6), human basophil cell line (KU812), human B lymphoblast (SUP-B15), human epithelial kidney cortex cells (WT 9-7, WT 9-12).

As an alternative, uptake or internalization of polypeptides can be determined by using porcine intestinal tissue in a gut ligated loop experiment, as is disclosed in the present examples. These data support the efficient transport of the APN-targeted antibodies across the small intestinal epithelium.

Because APN sequences are known to exist in cells from various species, the endogenous gene may be modified to permit, or increase, expression of the APN polypeptide. Cells can be modified (e.g., by homologous recombination) to provide increased expression by replacing, in whole or in part, the naturally occurring APN promoter with all or part of a heterologous promoter, so that the cells express APN polypeptide at higher levels. Alternatively, APN expression may also be induced by treatment with compounds known to induce expression of APN in a cell, such as for example a treatment with basic fibroblast growth factor (bFGF), or with bestatin.

In one embodiment, the polypeptide of the invention, is said to be “cross-reactive” for two different antigens or antigenic determinants (such as e.g., APN from different species of mammal, such as e.g. human APN, pig APN, dog APN, cat APN, horse APN, bovine APN, rat APN, mouse APN, and/or rhesus APN) if it is specific for (as defined herein) these different antigens or antigenic determinants. It will be appreciated that a polypeptide, in particular an antibody or antigen-binding fragment thereof, may be considered to be cross-reactive although the binding affinity for the two different antigens can differ, such as by a factor, 2, 5, 10, 50, 100 or even more, provided it is specific for (as defined herein) these different antigens or antigenic determinants.

The present invention provides isolated antigen-binding polypeptides, in particular antibodies and antigen-binding fragments thereof, that specifically bind to aminopeptidase N (APN) or an antigenic fragment thereof.

As used herein, the term “isolated” or “purified” in association with a polypeptide or nucleic acid means that the polypeptide or nucleic acid is not in its natural medium or in its natural form. Thus, the term “isolated” includes polypeptides or nucleic acids taken from the original environment, for example, if it is naturally occurring. For example, an isolated polypeptide generally does not contain at least some proteins or other cellular components that it is usually bound to or usually mixed with or in solution. Isolated polypeptides include the naturally produced polypeptides contained in cell lysates, the polypeptides in purified or partially purified form, recombinant polypeptides, the polypeptides expressed or secreted by cells, and in heterologous host cells or cultures of the polypeptide. In connection with nucleic acids, the term isolated or purified indicates that the nucleic acid is not in its natural genomic background (e.g., in a vector, as an expression cassette, linked to a promoter, or artificially introduced into a heterologous host cell).

The term “antibody”, as used herein, refers to immunoglobulin molecules comprising four polypeptide chains, two heavy chains (HCs) and two light chains (LCs) inter-connected by disulfide bonds (i.e., “full antibody molecules”), as well as multimers thereof (e.g. IgM). Each heavy chain comprises a heavy chain variable region (“HCVR” or “VH”) and a heavy chain constant region (comprised of domains CH1, CH2 and CH3). Each light chain is comprised of a light chain variable region (“LCVR or “VL”) and one light chain constant region (CL). The light or heavy chain variable region is composed of three hypervariable regions called “complementarity determining regions” or “CDRs” and a framework region that separates them. The framework region (FR) of the antibody, that is, the framework region that constitutes the combination of the light chain and the heavy chain, plays a role of locating and aligning CDRs, which are mainly responsible for binding to the antigen.

Each VH and VL comprises three CDRs and four FRs, arranged from amino-terminus to carboxy-terminus in the following order: FR1, CDR1, FR2, CDR2, FR3, CDR3, FR4. Heavy chain CDRs can also be referred to as HCDRs or CDR-Hs, and numbered HCDR1, HCDR2, and HCDR3 (or CDR-H1, CDR-H2, and CDR-H3). Likewise, light chain CDRs can be referred to as LCDRs or CDR-Ls, and numbered LCDR1, LCDR2, and LCDR3 (or CDR-L1, CDR-L2, and CDR-L3).

The term “antibody” includes polyclonal antibodies and monoclonal antibodies, but in particular monoclonal antibodies, and antigen-binding fragments (also referred to as antibody fragments or fragments) of these antibodies, including recombinant antibodies, single chain antibodies, single domain antibodies (also referred to as nanobodies or VHH antibodies), Fab, Fab′, F(ab′)2, Fv and scFv. The type of antibody can be IgG1, IgG2, IgG3, IgG4, IgA, IgM, IgE, IgD. In addition, the term “antibody” includes, purified antibodies, naturally occurring antibodies as well as non-naturally occurring antibodies, including, for example, chimeric, bifunctional, and humanized antibodies, and related synthetic isomeric forms (isoforms).

In the context of the present invention, the term “nanobody” or “VHH antibody” is to be understood as an antigen-binding fragment of heavy chain only antibodies. A nanobody has a very small size of around 15 kDa and is more stable and robust than a whole antibody. The advantage of these antibody-derived molecules is their small size which enables their binding to hidden epitopes not accessible to whole antibodies. In the context of therapeutic applications, a small molecular weight also means a rapid renal clearance and an efficient tissue penetration in for example tumors or through the blood brain barrier.

In an embodiment of the invention, an APN-binding polypeptide, e.g., antibody or antigen-binding fragment comprises a heavy chain constant domain, e.g., of the type IgA (e.g., IgA1 or IgA2), IgD, IgE, IgG (e.g., IgG1, IgG2, IgG3 and IgG4) or IgM. In an embodiment of the invention, an APN-binding polypeptide, e.g. antibody or antigen-binding fragment comprises a light chain constant domain, e.g. of the type kappa or lambda.

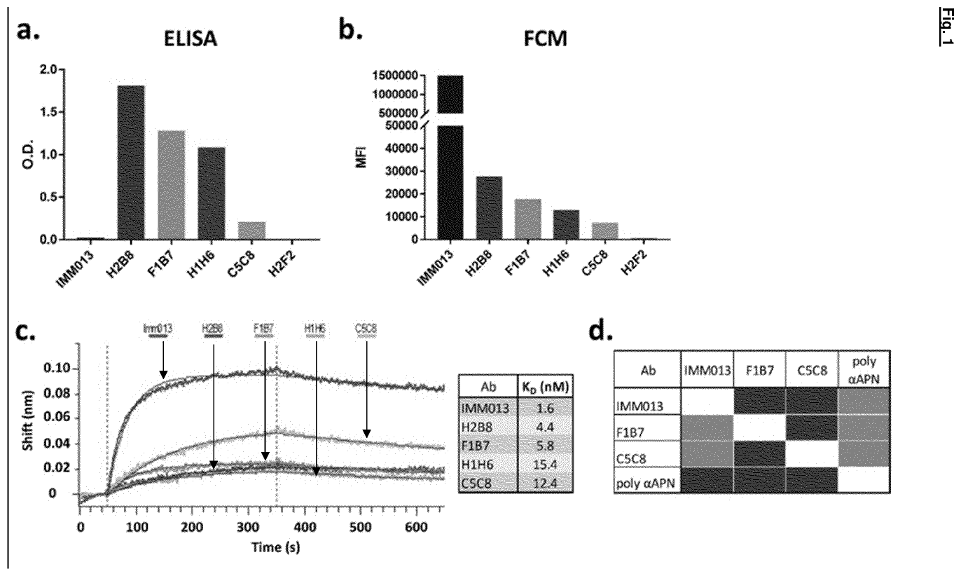

An amino acid sequence (such as antibody or fragment of the invention, or generally an antigen-binding protein or polypeptide) that can “bind to” or “specifically bind to”, that “has affinity for” and/or that “has specificity for” a certain epitope, antigen or protein (or for at least one part, fragment or epitope thereof) is said to be “against” or “directed against” said epitope, antigen or protein or is a “binding” molecule with respect to such epitope, antigen or protein, or is said to be “anti”-epitope, “anti”-antigen or “anti”-protein (e.g., “anti”-APN). As used herein, the terms “specifically binding” or “specifically bind(s)” mean that a polypeptide exhibits significant affinity for a particular protein or antigen (in the context of the present invention: APN) and, generally, does not exhibit significant reactivity with proteins or antigens other than APN. “Significant affinity” includes binding with an affinity of about 10⁻⁶ M (KD) or stronger; such as about or stronger then 10⁻⁷ M (KD), 10⁻⁸ M, 10⁻⁹ M, 10⁻¹⁰ M, 10⁻¹¹ M, or 10⁻¹² M. Preferably, binding is considered specific when binding affinity is about 10⁻¹² to 10⁻⁸ M, 10⁻¹² to 10⁻⁹ M, 10⁻¹¹ to 10⁻⁸ M, preferably of about 10⁻¹¹ to 10⁻⁹ M. Whether a polypeptide specifically reacts with or binds to a target can be tested readily by, inter alia, comparing the reaction of said polypeptide with a target protein or antigen (in the context of the present invention: APN) with the reaction of said polypeptide with proteins or antigens other than APN. Preferably, an antibody or fragment of the invention does not essentially bind or is not capable of binding to proteins or antigens other than APN. In one embodiment, the invention provides a polypeptide, in particular an antibody or antigen-binding fragment thereof, specifically binding APN with a binding affinity between about 10⁻¹² to 10⁻⁸ M, about 10⁻¹² to 10⁻⁹ M, about 10⁻¹¹ to 10⁻⁸ M, more specifically between about 10⁻¹¹ to 10⁻⁹ M, even more specifically with a binding affinity of about 4×10⁻⁹ M or less, about 3×10⁻⁹ M or less, about 2×10⁻⁹ M or less, preferably about 4×10⁻⁹ M or less, as determined by bio-layer interferometry (BLI). Other antibodies described in the context of the present invention that serve as a comparative example have a binding affinity that is significantly higher compared to the antibody or antigen-binding fragment thereof of the invention (see FIG. 1C) and are in the range of about 5×10⁻⁹ M or higher.

The present invention further includes anti-APN chimeric antibodies and antigen-binding fragments thereof, and methods of use thereof. As used herein, a “chimeric antibody” is well known to the skilled person and includes an antibody having the variable domain from a first antibody and the constant domain from a second antibody, where the first and second antibodies are from different species. Thus, when the term antibody or antigen-binding fragment thereof is used, it can also be a chimeric antibody. Specific examples are disclosed herein below.

In a further embodiment, the present invention provides a “chimeric molecule” (optionally also referred to as a construct) comprising at least one polypeptide as defined herein, in particular at least one antibody or antigen-binding fragment thereof as provided herein, or a chimeric antibody as provided herein, coupled, linked or conjugated (directly or indirectly) to at least one compound having a biological or functional activity, such as a chemical (e.g. small molecules) or biological, in particular a drug/therapeutic, a bio-active compound, an antigen, a toxin, and/or a diagnostic (e.g. a label, marker or imaging agent). In a specific embodiment, the present invention provides a chimeric molecule comprising the antibody or antigen-binding fragment thereof, or a chimeric antibody, and a compound, in particular a bio-active compound, more in particular an antigen. In said embodiment, the polypeptide or antibody will act as a carrier for the delivery of said compound across the intestinal barrier. The drug or therapeutic will be active in the intestinal submucosa or in the intestinal mucosa-associated lymphoid tissue and/or the antigen will induce an immune response in the intestinal submucosa or in the intestinal mucosa-associated lymphoid tissue. Said therapeutic agent may be an anti-inflammatory, anticancer, cytotoxic, anti-infective (e.g., anti-fungal, antibacterial, anti-parasitic, anti-viral, a toxin, a cytotoxic drug, a radionuclide, etc.) agent. Antigens include, but are not limited to proteins, peptides, lipids, nucleic acids, glycolipids and glycoproteins, carbohydrates, oligosaccharides, and polysaccharides. Also different drug delivery systems or (drug-loaded) carriers e.g. based on nanoparticles (NPs) can be conjugated to the polypeptide of the invention, including inorganic, magnetic, and polymeric particles or NPs.

In another specific embodiment, the present invention provides a chimeric molecule, or an antibody or antigen-binding fragment thereof; wherein said antibody or chimeric molecule binds aminopeptidase N with a binding affinity between about 10⁻¹² to 10⁻⁸ M, 10⁻¹¹ to 10⁻⁸ M, more specifically between about 10⁻¹¹ to 10⁻⁹ M, even more specifically with a binding affinity of about 4×10⁻⁹ M or less, about 3×10⁻⁹ M or less, about 2×10⁻⁹ M or less, preferably about 4×10⁻⁹ M or less.

In one embodiment, the antibody or fragment of the invention is conjugated to the tip adhesin FedF of F18 fimbriae, more specific to amino acids 15 to 165 of FedF. F18 fimbriae are composed of the major structural subunit FedA and several minor subunits including FedF. The latter is located at the tip of the fimbriae and is crucial for adherence of F18 fimbriated E. coli to fucosylated glycosphingolipids present in the apical membrane of small intestinal epithelial cells (Coddens et al., 2009).

### SEQ FedF₁₅₋₁₆₅:

(SEQ ID NO: 41)
NSSASSAQVTGTLLGTGKTNTTQMPALYTWQHQIYNVNFIPSSSGTLT
CQAGTILVWKNGRETQYALECRVSIHHSSGSINESQWGQQSQVGFGTA
CGNKKCRFTGFEISLRIPPNAQTYPLSSGDLKGSFSLTNKEVNWSASI
YVPAIAK

In another embodiment, the antibody or fragment of the invention is conjugated to the major structural subunit FedA of F18 fimbriae.

### SEQ FedA:

(SEQ ID NO: 42)
NLTPQISGTVGDTIQLGTVAPSGAGREIPFALKASSNVGGCASLSTKT
ADITWSGQLTEKGFANQGGVANDSYVALKTVNGKTQGQEVKASNSTVS
FDASKATTDGFKFTAQLKGGQTPGDFQGAAAYAVTYKGGGGGGQQGDV
KFFGNV

In yet another embodiment, the antibody or fragment of the invention is conjugated to the major structural subunit and adhesin FaeG of F4 fimbriae.

### SEQ FaeGac:

(SEQ ID NO: 43)
WMTGDFNGSVDIGGSITADDYRQKWEWKVGTGLNGFGNVLNDLTNGGT
KLTITVTGNKPILLGRTKEAFATPVTGGVDGIPHIAFTDYEGASVVLR
NPDGETNKKGLAYFVLPMKNAEGTKVGSVKVNASYAGVLGRGGVTSAD
GELLSLFADGLSSIFYGGLPRGSELSAGSAAAARTKLFGSLSRNDILG
QIQRVNANITSLVDVAGSYRENMEYTDGTVVSAAYALGIANGQTIEAT
FNQAVTTSTQWSAPLNVAITYY

The term “conjugated to”, as used herein, refers, in particular, to chemical and/or enzymatic conjugation resulting in a stable covalent link. Coupling to obtain the chimeric molecule can occur via a specific amino acid (e.g. lysine, cysteine) present in the antibody. As already mentioned, any moiety can be coupled, such as agents (e.g. proteins, nucleotide sequences, lipids, carbohydrates, peptides, drug moieties (e.g. cytotoxic drugs, antibody drug-conjugates or payload), tracers and detection agents) with a particular biological or specific functional activity. In the case where the coupled moiety is a genetically encoded therapeutic or diagnostic protein or nucleotide sequence, the coupled moiety may be synthesized or expressed by either peptide synthesis or recombinant DNA methods that are well known in the art. In another aspect, where the conjugated moiety is a non-genetically encoded peptide, e.g. a drug moiety, the conjugated moiety may be synthesized artificially or purified from a natural source.

In a further embodiment, the polypeptide or the chimeric molecule as provided herein can further comprise one or more other groups, residues, moieties or binding units. The one or more other groups, residues, moieties or binding units are preferably chosen from the group consisting of a polyethylene glycol molecule, serum proteins or fragments thereof, binding units that can bind to serum proteins, an Fc portion or small proteins or peptides that can bind to serum proteins, further amino acid residues, tags or other functional moieties, e.g., toxins, labels, radiochemicals, etc. The other groups, residues, moieties or binding units may for example be chemical groups, residues, moieties, which may or may not by themselves be biologically and/or pharmacologically active. For example, and without limitation, such groups may be linked to the one or more antibodies of the invention so as to provide a “derivative” of the polypeptide or construct of the invention.

In general, various linkers known in the art can be used to link the antibody or fragment and the (bio-active) compound, agent or moiety according to the invention. As should be clear, cleavable and non-cleavable linkers can be employed to achieve the desired release profile. In general, the optimal combination of linker and conjugation chemistry must be uniquely tailored to correlate each unique facet: the antibody or fragment, the conjugated moiety, and the profile of the disease to be treated. Still other suitable spacers or linkers will be clear to the skilled person, and may generally be any linker or spacer used in the art. In specific aspects the linkers or spacers are suitable for use in applications which are intended for pharmaceutical use. For example, a linker between the lysine and the conjugate may in certain aspects also be a suitable amino acid sequence, and in particular amino acid sequences of between 1 and 50, or more specifically, between 1 and 30 amino acid residues. Some examples of such amino acid sequences include Gly-Ser (GS) linkers. Still other suitable linkers generally comprise organic compounds or polymers, in particular those suitable for use in polypeptides for pharmaceutical use. For instance, poly(ethyleneglycol) moieties have been used to link antibody domains. It is encompassed within the scope of the invention that the length, the degree of flexibility and/or other properties of the linker may have some influence on the properties of the final antibody conjugate of the invention, including but not limited to the affinity, specificity or avidity for a specific target. Based on the disclosure herein, the skilled person will be able to determine the optimal linker for use in a specific antibody of the invention, optionally after some limited routine experiments.

Several approaches to ensure the prolongation of half-life as known to the skilled person are considered embodiments of the present invention. Hence, the efficacy of the current polypeptides can be improved by fine-tuning the residence time. The polypeptides provided herein can be chemically modified in order to increase their molecular weight. Such a chemical modification, for example with a polyethylene glycol (PEG) group, may also protect against proteases. Another strategy to extend the half-life is by coupling the polypeptide to long-lived serum proteins or to building blocks that target these long-lived proteins. Furthermore, it has been observed that negatively charged, small proteins remain longer in circulation than neutral proteins. There are several ways to add negative charges to proteins including the addition of sialic acid polymers (polysialylation) or hydroxyethal starch (HESylation) and by fusion with the highly sialylated beta carboxyterminal peptide (CTP) amino acid-residue found in the human chorionic gonadotropin (hCG) hormone.

Finally, ‘Fc domain-based fusion constructs’ are also widely used to extend the half-life of therapeutic proteins, including antibodies or fragments. Hence, a further strategy is to fuse the polypeptide of the invention to the Fc region or other Fc-moieties of an IgG molecule. However, other Fc domains are also qualified, such as the Fc domain of IgA which is more stable in the intestinal environment. Suitable Fc domains and methods for the production of Fc domain-based fusion constructs are well known to the skilled person.

For example, the antibody or fragments of the present invention may be fused to the Fc domain of mouse IgG, more specifically mouse IgG1. Antibodies or fragments of the present invention fused to the Fc domain of pig IgG or pig IgA or an Fc receptor binding moiety may also be part of the present invention.

In one embodiment, the present invention provides a chimeric antibody comprising the variable domains of at least one polypeptide, in particular an antibody or fragment, as provided herein, and the constant domains of the light (LCCR) and heavy chain (HCCR), in particular the LCCR and HCCR of IgG or IgA, with sequences derived from but not limited to human, pig, dog, cat, horse, bovine, rat, mouse, rhesus, etc. In a specific embodiment, also referred to herein as α-APN-mIgG1-FedF fusion construct, the invention provides a polypeptide comprising or consisting of SEQ ID NO: 2 and optionally SEQ ID NO: 12 and the constant domain of mouse light chain and the constant domains of the heavy chain of mouse IgG (anti-APN-mIgG) comprising at least part of the sequence of respectively SEQ ID NO: 10 and SEQ ID NO: 20 or a sequence having at least 95%, 96%, 97%, 98%, 99% identity thereto. The present invention may also provide chimeric antibodies wherein the constant domain sequences are replaced by the respective porcine sequences of IgA (anti-APN-pIgA) or IgG1 to IgG6 (anti-APN-pIgG1 to 6), e.g. as presented in Table 5, or a part thereof or a sequence having at least 95%, 96%, 97%, 98%, 99% identity thereto. Examples are the α-APN-pIgA or α-APN-pIgG fusion constructs as disclosed herein.

The present invention also provides an isolated nucleic acid encoding the aforementioned APN-binding polypeptides, in particular antibodies and antigen-binding fragments thereof, or encoding the chimeric antibody or chimeric molecule. In this context, the nucleic acid contains variants of its conservative substitutions (e.g. substitution of degenerate codons) and complementary sequences. The terms “nucleic acid” and “polynucleotide” are synonymous and include genes, cDNA molecules, mRNA molecules, and fragments thereof such as oligonucleotides.

The present invention also provides a vector including the above-mentioned nucleic acid. The nucleic acid sequence is operably linked to at least one regulatory sequence. “Operably linked” means that the coding sequence is linked to the regulatory sequence in a manner that allows expression of the coding sequence. Regulatory sequences are selected to direct the expression of the protein of interest in a suitable host cell, and include promoters, enhancers, and other expression control elements. Hence, in one embodiment, the vector includes a promoter for driving expression of the nucleic acid disclosed herein, optionally a nucleic acid sequence encoding a signal peptide (also referred to as leader sequence) that secretes or integrates the peptide expression product on the membrane, the nucleic acid of the present invention, and optional a nucleic acid sequence encoding a terminator. When the expression vector is manipulated in a production strain or cell line, the vector may or may not be integrated into the genome of the host cell when introduced into the host cell. The vector usually carries a replication site and a marker sequence that can provide phenotypic selection in the transformed cell.

In this context, a vector may refer to a molecule or agent that contains the nucleic acid or a fragment thereof, is capable of carrying genetic information, and can deliver genetic information to cells. Typical vectors include plasmids, viruses, bacteriophages, cosmids, and mini-chromosomes. The vector can be a cloning vector (i.e. a vector used to transfer genetic information into a cell, the cell can be propagated and the cell can be selected with or without the genetic information) or an expression vector (i.e. contains the necessary genetic elements thereby allowing the genetic information of the vector to be expressed in the cell). Thus, a cloning vector may contain a selection marker and an origin of replication that matches the cell type specified by the cloning vector, while an expression vector contains the regulatory elements necessary to affect expression in the designated target cell.

The expression vector of the invention is used to transform host cells. Such transformed cells are also part of the present invention and can be cultured cells or cell lines used to propagate the nucleic acid and vectors provided herein, or to recombinantly prepare the polypeptides of the invention. The transformed cells of the present invention include microorganisms such as bacteria (e.g., Escherichia coli, Bacillus, etc.). Host cells also include cells from multicellular organisms such as fungi, insect cells, plant cells or mammalian cells, preferably cells from mammals, such as Chinese hamster ovary (CHO) cells. Further examples of host cells include NS0, SP2 cells, HeLa cells, baby hamster kidney (BHK) cells, monkey kidney cells (COS), human hepatocellular carcinoma cells (e.g., Hep G2), A549 cells, 3T3 cells, and HEK-293 cells, Pichia sp., Saccharomyces sp., Hansenula polymorpha, Kluyveromyces sp., etc. The transformed cell can replicate the nucleic acid provided herein. When recombinantly preparing the polypeptide of the present invention, the expression product may be exported to the culture medium or carried on the surface of the transformed cell. Introduction of the vector in host cells can be effected by, but not limited to, calcium phosphate transfection, virus infection, DEAE-dextran mediated transfection, lipofectamin transfection or electroporation, and any person skilled in the art can select and use an introduction method suitable for the expression vector and host cell used.

In one embodiment, the present invention includes an isolated host cell (e.g., a CHO cell) comprising an (chimeric) antibody of the invention, such as the antibodies comprising sequences as disclosed in Tables 2 or 4; or a fragment thereof, or a nucleic acid encoding such a polypeptide (Tables 3 or 5) or a signal peptide.

In the context of the present invention, the term “signal peptide”, also referred to as “signal sequence”, “targeting signal”, “localization signal”, “localization sequence”, “transit peptide”, “leader sequence” or “leader peptide”, is a short peptide (usually 16-30 amino acids long) present at the N-terminus (or occasionally C-terminus) of most newly synthesized proteins that are destined toward the secretory pathway. Signal peptides function to prompt a cell to translocate the protein, usually to the cellular membrane.

Examples of a leader sequence and corresponding amino acid of the signal peptide (as used herein for aAPN-mIgG1-FedF) may be:

Heavy chain nucleic acid leader sequence:
(SEQ ID NO: 44)
ATGGGATTCAGCAGGATCTTTCTCTTCCTCCTGTCAATAACTACAGGT
GTCCACTCC
Heavy chain amino acid leader sequence:
(SEQ ID NO: 45)
MGFSRIFLFLLSITTGVHS
Light chain nucleic acid leader sequence:
(SEQ ID NO: 46)
ATGCATTTTCAAGTGCAGATTTTCAGCTTCCTGCTAATCAGTGCCTCA
GTCATCATGTCCCGAGGA
Light chain amino acid leader sequence:
(SEQ ID NO: 47)
MHFQVQIFSFLLISASVIMSRG

Other examples of a leader sequence and corresponding amino acid of the signal peptide (as used herein for aAPN-pIgA-FedF) may be:

Heavy chain nucleic acid leader sequence:
(SEQ ID NO: 48)
ATGGAGTTTGGCCTGTCCTGGGTGTTCCTGGTGGCCCTGTTTCGCGGA
GTGCAGTGC
Heavy chain amino acid leader sequence:
(SEQ ID NO: 49)
MEFGLSWVFLVALFRGVQC
Light chain nucleic acid leader sequence:
(SEQ ID NO: 50)
ATGAAGTACCTGCTGCCCACAGCTGCTGCTGGACTGCTGCTGCTGGCT
GCTCAGCCTGCTATGGCC
Light chain amino acid leader sequence:
(SEQ ID NO: 51)
MKYLLPTAAAGLLLLAAQPAMA

In one embodiment, other signal peptides may be used that are well known to the skilled person for example as described in Haryadi R et al., 2015, PloS One, or for example IL-2 or non eukaryotic signal peptides.

In a further embodiment, the present invention includes recombinant methods for making an anti-APN antigen-binding polypeptide, such as an antibody or antigen-binding fragment thereof of the present invention, or an immunoglobulin chain thereof, comprising (i) introducing one or more nucleic acid sequences, in particular sequences including the nucleotide sequence of any one or more of the sequences of Tables 3 or 5, encoding light and/or heavy immunoglobulin chains, or CDRs, of the polypeptide, in particular amino acid sequences as disclosed in Tables 2 or 4, for example, wherein the nucleic acid is in a vector; and/or integrated into a host cell chromosome and/or is operably linked to a promoter; (ii) culturing the host cell (e.g., CHO or Pichia or Pichia pastoris) under conditions favorable to expression of the nucleic acid and, (iii) optionally, isolating the antigen-binding polypeptide, (e.g., antibody or fragment) or chain from the host cell and/or medium in which the host cell is grown. Hence, the invention also encompasses antibodies or fragments obtained by said method.

As used herein, the term “recombinant” antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell expression system, or a non-human cell expression system, or isolated from a recombinant combinatorial human antibody library. In some embodiments, a recombinant antibody shares a sequence with an antibody isolated from an organism (e.g., a human), but has been expressed via recombinant DNA technology. Such antibodies may have post-translational modifications (e.g., glycosylation) that differ from the antibody as isolated from the organism. There are several methods by which to produce recombinant antibodies which are known in the art.

In one embodiment, the SEQ ID numbers for the amino acid sequence for CDRs as well as for the VH and the VL region of exemplary antibodies of the invention, as well as the SEQ ID numbers for the nucleic acid sequences encoding them are listed in Tables 2, 3, 4 and 5.

In another embodiment, an antibody or antigen-binding fragment of the invention comprises or consists of a HCVR having an amino acid sequence that is at least and/or about 75%, 80%, 85%, 90%, 91%, 92%, 93%, 94%, 95%, 96%, 97%, 98%, 99% or 100% identical to the sequence recited in SEQ ID NO: 2. In another embodiment, an antibody or antigen-binding fragment of the invention comprises or consists of a LCVR having an amino acid sequence that is at least and/or about 75%, 80%, 85%, 90%, 91%, 92%, 93%, 94%, 95%, 96%, 97%, 98%, 99% or 100% identical to the sequence recited in SEQ ID NO: 12.

In an embodiment of the invention, the HCVR and/or LCVR of the anti-APN antibody or antigen-binding fragment can have a sequence variation as provided herein before, but the antibody or fragment comprises three, four, five, in particular six, CDRs of a single antibody as provided in Table 2 (e.g., HCDR1, HCDR2 and/or HCDR3; and/or LCDR1, LCDR2 and/or LCDR3). In particular an antibody, or antigen-binding fragment thereof, may comprise three heavy chain CDRs (HCDR1, HCDR2 and HCDR3) as set forth in resp. amino acid sequences SEQ ID NOs: 4, 6 and 8, and optionally one, two or three light chain CDRs (LCDR1, LCDR2 and LCDR3) as set forth in amino acid sequences resp. SEQ ID NOs: 14, 16 and 18. More in particular an antibody, or antigen-binding fragment thereof, may comprise three heavy chain CDRs (HCDR1, HCDR2 and HCDR3) as set forth in resp. amino acid sequences SEQ ID NOs: 4, 6 and 8, and three' light chain CDRs (LCDR1, LCDR2 and LCDR3) as set forth in amino acid sequences resp. SEQ ID NOs: 14, 16 and 18.

In a further embodiment, the invention provides a polypeptide comprising the three, four, five, in particular six, CDRs of a single antibody as provided in Table 2 (e.g., HCDR1, HCDR2 and/or HCDR3; and/or LCDR1, LCDR2 and/or LCDR3) and a light and heavy chain constant region of a single antibody as provided in Table 4.

More specific, the antibody, or antigen-binding fragment thereof, may comprise three heavy chain CDRs (HCDR1, HCDR2 and HCDR3) as set forth in resp. amino acid sequences SEQ ID NOs: 4, 6 and 8, three light chain CDRs (LCDR1, LCDR2 and LCDR3) as set forth in amino acid sequences resp. SEQ ID NOs: 14, 16 and 18, the light chain constant domain as set forth in amino acid sequence SEQ ID NO:58 and the heavy chain constant domain as set forth in amino acid sequence SEQ ID NO: 59-70.

In yet another embodiment, an antibody or antigen-binding fragment of the invention comprises or consists of a heavy chain or heavy chain constant region (HCCR) having an amino acid sequence that is at least and/or about 75%, 80%, 85%, 90%, 91%, 92%, 93%, 94%, 95%, 96%, 97%, 98%, 99% or 100% identical to the sequence recited in respectively SEQ ID NO: 1, 10, 59-70. In a further embodiment, an antibody or antigen-binding fragment of the invention comprises or consists of a light chain or light chain constant region (LCCR) having an amino acid sequence that is at least and/or about 75%, 80%, 85%, 90%, 91%, 92%, 93%, 94%, 95%, 96%, 97%, 98%, 99% or 100% identical to the sequence recited in respectively SEQ ID NO: 11, 20 or 58.

In yet another embodiment, the heavy chain, heavy chain variable region (HCVR), or heavy chain constant region (HCCR) of an antibody of the invention may be encoded by a nucleic acid that has a sequence that is at least and/or about 70%, 75%, 80%, 85%, 90%, 91%, 92%, 93%, 94%, 95%, 96%, 97%, 98%, 99% or 100% identical to the sequence recited in respectively SEQ ID NO: 21, 22 and 30. In another embodiment, the heavy chain constant region of the chimeric antibody of the invention may be encoded by a nucleic acid that has a sequence that is at least and/or about 70%, 75%, 80%, 85%, 90%, 91%, 92%, 93%, 94%, 95%, 96%, 97%, 98%, 99% or 100% identical to the sequence recited in SEQ ID NO: 72-83.

In yet another embodiment, the light chain, light chain variable region (LCVR), or light chain constant region (LCCR) of an antibody of the invention may be encoded by a nucleic acid that has a sequence that is at least and/or about 70%, 75%, 80%, 85%, 90%, 91%, 92%, 93%, 94%, 95%, 96%, 97%, 98%, 99% or 100% identical to the sequence recited in respectively SEQ ID NO: 31, 32 and 40. In another embodiment, the light chain constant region of the chimeric antibody of the invention may be encoded by a nucleic acid that has a sequence that is at least and/or about 70%, 75%, 80%, 85%, 90%, 91%, 92%, 93%, 94%, 95%, 96%, 97%, 98%, 99% or 100% identical to the sequence recited in respectively SEQ ID NO: 71.

The present invention also provides an isolated nucleic acid encoding the aforementioned APN-binding polypeptides, in particular antibodies and antigen-binding fragments thereof comprising three heavy chain CDRs (HCDR1, HCDR2 and HCDR3) as set forth in resp. nucleic acid sequences SEQ ID NOs: 24, 26 and 29, and optionally one, two or three light chain CDRs (LCDR1, LCDR2 and LCDR3) as set forth in nucleic acid sequences resp. SEQ ID NOs: 34, 36 and 38.

The present invention also provides an isolated nucleic acid encoding the aforementioned APN-binding polypeptides, in particular antibodies and antigen-binding fragments thereof comprising three heavy chain CDRs (HCDR1, HCDR2 and HCDR3) as set forth in resp. nucleic acid sequences SEQ ID NOs: 24, 26 and 29, and three light chain CDRs (LCDR1, LCDR2 and LCDR3) as set forth in nucleic acid sequences resp. SEQ ID NOs: 34, 36 and 38.

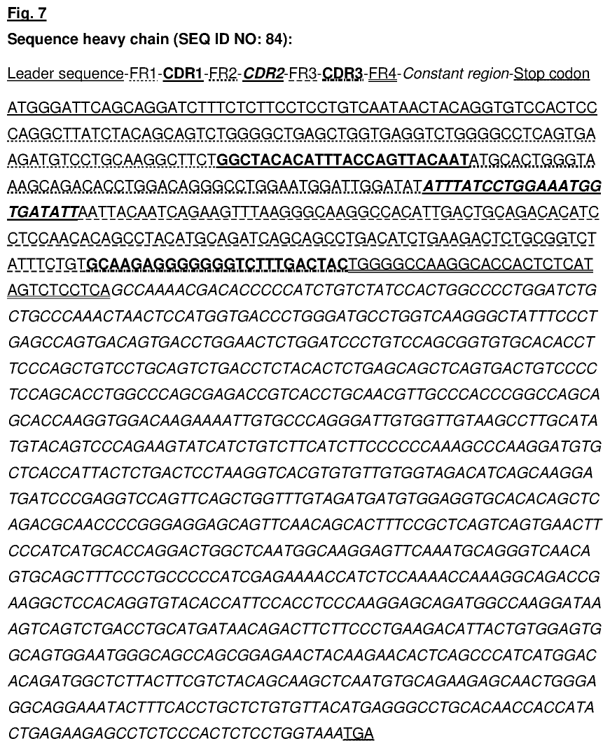

As an example, a nucleic acid sequence is provided in FIG. 7 (SEQ ID NOs: 84 and 85).

For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position). Alternatively, the degree of sequence identity between two or more nucleotide sequences may be calculated using a known computer algorithm for sequence alignment such as NCBI Blast v2.0, using standard settings.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings. A specific method utilizes the BLAST module of WU-BLAST-2 set to the default parameters, with overlap span and overlap fraction set to 1 and 0.125, respectively.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, lie, Val and Cys; and (e) aromatic residues: Phe, Tyr and Trp. Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into lie or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu. Hence, in one embodiment, a sequence having a given percentage sequence identity as given herein before is a sequence having one, two, three or more conservative amino acid substitutions as compared to the reference sequence.

In a specific embodiment, amino acid sequence modifications of the antibody or fragment described herein are contemplated. For example, it may be desirable to improve the binding affinity and/or other biological properties of the antibody. Amino acid sequence variants of the antibody are prepared by introducing appropriate nucleotide changes into the antibody constructs nucleic acid, or by peptide synthesis.

In one embodiment, the anti-APN antibody or fragment comprises or consists of an HCVR and an LCVR comprising an amino acid sequence provided in Table 2.

An antigen-binding fragment of an antibody will, in an embodiment of the invention, comprise at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In a further embodiment, an antigen-binding fragment of an antibody may contain at least one variable domain covalently linked to at least one constant domain. The variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule.

In one embodiment, the anti-APN antibody or chimeric antibody comprises or consists of a HCVR and a LCVR with the amino acid sequence provided in Table 2, and an amino acid sequence encoded by the following nucleic acid sequence constant regions provided in Table 3 (Heavy chain constant region (HCCR): SEQ ID NO: 30; and Light chain constant region (LCCR): SEQ ID NO: 40.) or as provided in Table 5 (HCCR: SEQ ID NO: 72-83; LCCR: SEQ ID NO: 71.

In one embodiment, the present invention provides an (chimeric) antibody or antigen-binding fragment thereof that specifically binds to epitopes in the aminopeptidase N protein. In one embodiment, the present invention provides an (chimeric) antibody or antigen-binding fragment thereof wherein the (chimeric) antibody or fragment thereof upon binding to epitopes of the aminopeptidase N protein induces conformational changes in the protein structure. In particular, specific three-dimensional structural characteristics, and/or specific charge characteristics may change upon binding of the (chimeric) antibody or antigen-binding fragment thereof; resulting in a conformational structure effect that leads to the masking of other epitopes.

In a particular embodiment, the epitopes of the aminopeptidase N protein may comprise or consist of the amino acid sequence LLCQEPTDVIIIHS (SEQ ID NO: 52), FQSNETAQNGVLIRIW (SEQ ID NO: 53), ALNVTGPILNFFAN (SEQ ID NO: 54), PQSCSISNKERVVTVIA (SEQ ID NO: 55), LPDTVRAIMDRWTLQMGFPVIT (SEQ ID NO: 56), QHQLQTNLSVIPVI (SEQ ID NO: 57) or combinations thereof, or a sequence having at least 92%, 93%, 94%, 95%, 96%, 97%, 98%, 99% identity thereto.

In a specific embodiment, the epitope of the aminopeptidase N protein comprises or consists of amino acid sequences FQSNETAQNGVLIRIW (SEQ ID NO: 53), ALNVTGPILNFFAN (SEQ ID NO: 54), PQSCSISNKERVVTVIA (SEQ ID NO: 55) or a sequence having at least 92%, 93%, 94%, 95%, 96%, 97%, 98%, 99% identity thereto.

In one embodiment, the present invention provides an (chimeric) antibody or antigen-binding fragment thereof wherein the (chimeric) antibody or fragment specifically binds an epitope in the aminopeptidase N protein comprising at least one of the following amino acids sequence SEQ ID NO: 52, 53, 54, 55, 56, or 57 or a sequence having at least 92% identity hereto.

In a further embodiment, the present invention provides an (chimeric) antibody or antigen-binding fragment thereof wherein the (chimeric) antibody or fragment specifically binds an epitope in the aminopeptidase N protein comprising at least two of the amino acid sequences SEQ ID NO: 53, 54, or 55 or sequences having at least 92% identity, hereto.

As described herein, amino acid sequences SEQ ID NO: 53, 54 and 55 may be separate epitopes of the aminopeptidase N protein or form subregions of one epitope of the aminopeptidase N protein. More specifically, the subregion may be formed by combinations of SEQ ID NO: 53, 54 and 55; or SEQ ID NO: 53 and 54; or SEQ ID NO: 53 and 55; or SEQ ID NO: 54 and 55, depending on the conformational structure of the aminopeptidase N protein.

In a further embodiment, the epitope amino acid sequence of the aminopeptidase N protein comprises an amino acid sequence of swine, mouse, equus, human, hamster, monkey, feline, cattle, in particular swine.

As used herein, the term “epitope” refers to an antigenic determinant (e.g., an APN polypeptide) that interacts with a specific antigen-binding site of an antigen-binding protein, e.g., a variable region of an antibody molecule, known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Methods for determining the epitope of an antibody or fragment are well known to the skilled person and include for example alanine scanning mutational analysis, peptide blot analysis, peptide cleavage analysis, crystallographic studies and NMR analysis. In the present invention epitopes were determined using a linear (pepscan) and conformational epitope mapping strategy (chemically linked peptides on scaffolds: CLIPS), which are a well-known procedures for mapping and characterizing epitopes involving the synthesis of overlapping peptides and analysis of the peptides in enzyme-linked immunosorbent assays (ELISAs).

In a further aspect, the present invention also provides a pharmaceutical composition comprising a polypeptide, in particular the (chimeric) antibody or fragment, a chimeric molecule, a nucleic acid, a vector, a host cell—each described herein—and further comprising at least one a pharmaceutically acceptable diluent and/or carrier.

In a further embodiment, the current invention also provides a polypeptide, in particular the (chimeric) antibody or fragment, chimeric molecule, the nucleic acid, the vector, the host cell or a pharmaceutical composition provided herein for use in human or veterinary medicine, more in particular for use in preventing, treating and/or reducing symptoms of an intestinal disease, even more in particular for use in oral vaccination against intestinal disease. In the context of the present invention, the term ‘preventing’ or alternatively ‘to prevent’ are meant to be to ‘stop’, to ‘avert’, to ‘arrest’, to ‘block’, or to ‘halt’, completely (i.e. 100%) reduce symptoms of a disease. In the context of the present application, the terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptoms but has not yet been diagnosed as having it; (b) inhibiting the disease symptoms, i.e. arresting its development; or (c) relieving the disease symptom, i.e. causing regression of the disease or symptom. ‘Intestinal disease’ as used herein refers to gut or enteric infections and inflammatory disease of the gut or intestines, more specific the small intestines (e.g. inflammatory bowel disease, Crohn's disease or ulcerative colitis). Such Inflammation can e.g. be caused by an autoimmune disease. Infections with intestinal pathogens include infection with a species selected from the group of genera consisting of: pathogenic Escherichia coli (ETEC, EHEC, EPEC, STEC, . . . ), Vibrio, Campylobacter, Clostridium, Salmonella, Yersinia, Lawsonia, Rotavirus, Shigella, PEDV, Cryptosporidium, and Isospora. In particular, the invention provides a method for treating and/or preventing an intestinal disease by administering a polypeptide, chimeric molecule or pharmaceutical composition of the invention to a subject. In one embodiment, administration is orally. The polypeptide, in particular antibody or fragment thereof, as provided herein can function as a carrier, targeting a compound, in particular a bio-active compound, more in particular an antigen, across the (gastro)intestinal mucosa. In one aspect, the present invention focuses on the polypeptide as a carrier for targeted vaccine delivery to the gut epithelium to induce strong mucosal immune responses, e.g. against gut pathogens. As used herein, “mucosal immune response” refers to an immune response (humoral and cellular response) that occurs at a mucosal membrane of the intestine, the urogenital tract and/or the respiratory system, i.e., surfaces that are in contact with the external environment. In the context of the present invention, the mucosal immune response is an intestinal mucosal immune response occurring at a mucosal membrane of the small intestine, in particular the production of immunoglobulin A (IgA). The invention furthermore provides a method for treating an intestinal disease and/or to induce an immune response against an intestinal pathogen in a subject by orally administering the polypeptide, chimeric molecule or composition as provided herein to the subject.

In another embodiment, the invention relates to the use of a polypeptide, in particular (chimeric) antibody or fragment, and/or chimeric molecule of the invention in the preparation of a pharmaceutical composition. Furthermore, the invention provides the use of an (chimeric) antibody or antigen-binding fragment thereof, the nucleic acid, the vector, the host cell, or the pharmaceutical composition (i) in the manufacture of a medicament for the treatment of an intestinal disease, (ii) in a vaccine, or (iii) in diagnosis. Hence, also provided is a pharmaceutical composition and its use in one or more of the methods of treatment mentioned herein. Typically, the pharmaceutical composition comprises the polypeptide and/or construct as described herein and a pharmaceutically acceptable carrier and/or excipient, optionally in combination with an adjuvant.

A “carrier”, or “adjuvant”, in particular a “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and safe to a patient at concentrations consistent with effective activity of the active ingredient so that any side effects ascribable to the carrier do not impair the beneficial effects of the active ingredient. Preferably, a pharmaceutically acceptable carrier or adjuvant enhances the immune response elicited by an antigen. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles.

The polypeptides, and chimeric molecules of the invention and optionally a pharmaceutically acceptable carrier, diluent and/or excipient can be administered by any suitable route such as any of those commonly known to those of ordinary skill in the art. For therapy, the pharmaceutical composition of the invention can be administered to any subject in accordance with standard techniques. The administration can be by any appropriate mode, including orally, parenterally, topically, nasally, ophthalmically, intrathecally, intraventricularly, sublingually, rectally, vaginally, and the like, and preferably orally. Still other techniques of formulation as nanotechnology and aerosol and inhalant are also within the scope of this invention. The dosage and frequency of administration will depend on the age, sex and condition of the patient, concurrent administration of other drugs, counter-indications and other parameters to be taken into account by the clinician. In one embodiment, the treatment regimen is a prime-boost regimen. The dosage and concentration of the carrier, excipient and stabilizer should be safe to the subject (human, pigs, mice and other mammals), including buffers such as phosphate, citrate, and other organic acid; antioxidant such as vitamin C, small polypeptide, protein such as serum albumin, gelatin or immunoglobulin; hydrophilic polymer such as PVP, amino acid such as amino acetate, glutamate, asparagine, arginine, lysine; glycose, disaccharide, and other carbohydrate such as glucose, mannose or dextrin, chelate agent such as EDTA, sugar alcohols such as mannitol, sorbitol; counterions such as Na+, and/or surfactant such as TWEEN®, PLURONICS® or PEG and the like.

TABLE 2

| | | SEQ ID |
|---|---|---|
| Region | Amino acid sequence | NO |
| Heavy | QAYLQQSGAELVRSGASVKMSCKASGYTFTSYNMHWVKQTPGQGLEWIGYIYPGNGDI | 1 |
| chain | NYNQKFKGKATLTADTSSNTAYMQISSLTSEDSAVYFCARGGVFDYWGQGTTLIVSSAKT | |
| | TPPSVYPLAPGSAAQTNSMVTLGCLVKGYFPEPVTVTWNSGSLSSGVHTFPAVLQSDLY | |
| | TLSSSVTVPSSTWPSETVTCNVAHPASSTKVDKKIVPRDCGCKPCICTVPEVSSVFIFPPK | |
| | PKDVLTITLTPKVTCVVVDISKDDPEVQFSWFVDDVEVHTAQTQPREEQFNSTFRSVSEL | |
| | PIMHQDWLNGKEFKCRVNSAAFPAPIEKTISKTKGRPKAPQVYTIPPPKEQMAKDKVSLT | |
| | CMITDFFPEDITVEWQWNGQPAENYKNTQPIMDTDGSYFVYSKLNVQKSNWEAGNTFTC | |
| | SVLHEGLHNHHTEKSLSHSPGK | |
| HCVR | QAYLQQSGAELVRSGASVKMSCKASGYTFTSYNMHWVKQTPGQGLEWIGYIYPGNGDI | 2 |
| | NYNQKFKGKATLTADTSSNTAYMQISSLTSEDSAVYFCARGGVFDYWGQGTTLIVSS | |
| HC FR1 | QAYLQQSGAELVRSGASVKMSCKAS | 3 |
| HCDR1 | GYTFTSYN | 4 |
| HC FR2 | MHWVKQTPGQGLEWIGY | 5 |
| HCDR2 | IYPGNGDI | 6 |
| HC FR3 | NYNQKFKGKATLTADTSSNTAYMQISSLTSEDSAVYFC | 7 |
| HCDR3 | ARGGVFDY | 8 |
| HC FR4 | WGQGTTLIVSS | 9 |
| HCCR | AKTTPPSVYPLAPGSAAQTNSMVTLGCLVKGYFPEPVTVTWNSGSLSSGVHTFPAVLQS | 10 |
| | DLYTLSSSVTVPSSTWPSETVTCNVAHPASSTKVDKKIVPRDCGCKPCICTVPEVSSVFIF | |
| | PPKPKDVLTITLTPKVTCVVVDISKDDPEVQFSWFVDDVEVHTAQTQPREEQFNSTFRSV | |
| | SELPIMHQDWLNGKEFKCRVNSAAFPAPIEKTISKTKGRPKAPQVYTIPPPKEQMAKDKV | |
| | SLTCMITDFFPEDITVEWQWNGQPAENYKNTQPIMDTDGSYFVYSKLNVQKSNWEAGNT | |
| | FTCSVLHEGLHNHHTEKSLSHSPGK | |
| Light | QIVLTQSPAIVSASPGEKVTITCSGSSSVNYMHWFQQKPGTSPKLWIYSTSNLASGVPGR | 11 |
| chain | FSGSGSGTSYSLTISRMEAEDAATYYCQQRTSYPYTFGGGTKLEIKRADAAPTVSIFPPSS | |
| | EQLTSGGASVVCFLNNFYPKDINVKWKIDGSERQNGVLNSWTDQDSKDSTYSMSSTLTL | |
| | TKDEYERHNSYTCEATHKTSTSPIVKSFNRNEC | |
| LCVR | QIVLTQSPAIVSASPGEKVTITCSGSSSVNYMHWFQQKPGTSPKLWIYSTSNLASGVPGR | 12 |
| | FSGSGSGTSYSLTISRMEAEDAATYYCQQRTSYPYTFGGGTKLEIK | |
| LC FR1 | QIVLTQSPAIVSASPGEKVTITC | 13 |
| LCDR1 | SSVNY | 14 |
| LC FR2 | MHWFQQKPGTSPKLWIY | 15 |
| LCDR2 | STS | 16 |
| LC FR3 | NLASGVPGRFSGSGSGTSYSLTISRMEAEDAATYYC | 17 |
| LCDR3 | QQRTSYPYT | 18 |
| LC FR4 | FGGGTKLEIK | 19 |
| LCCR | RADAAPTVSIFPPSSEQLTSGGASVVCFLNNFYPKDINVKWKIDGSERQNGVLNSWTDQ | 20 |
| | DSKDSTYSMSSTLTLTKDEYERHNSYTCEATHKTSTSPIVKSFNRNEC | |

TABLE 3

| | | SEQ ID |
|---|---|---|
| Region | Nucleic acid sequence | NO |
| Heavy | CAGGCTTATCTACAGCAGTCTGGGGCTGAGCTGGTGAGGTCTGGGGCCTCAGTGAA | 21 |
| chain | GATGTCCTGCAAGGCTTCTGGCTACACATTTACCAGTTACAATATGCACTGGGTAAAG | |
| | CAGACACCTGGACAGGGCCTGGAATGGATTGGATATATTTATCCTGGAAATGGTGATA | |
| | TTAATTACAATCAGAAGTTTAAGGGCAAGGCCACATTGACTGCAGACACATCCTCCAA | |
| | CACAGCCTACATGCAGATCAGCAGCCTGACATCTGAAGACTCTGCGGTCTATTTCTGT | |
| | GCAAGAGGGGGGGTCTTTGACTACTGGGGCCAAGGCACCACTCTCATAGTCTCCTCA | |
| | GCCAAAACGACACCCCCATCTGTCTATCCACTGGCCCCTGGATCTGCTGCCCAAACT | |
| | AACTCCATGGTGACCCTGGGATGCCTGGTCAAGGGCTATTTCCCTGAGCCAGTGACA | |
| | GTGACCTGGAACTCTGGATCCCTGTCCAGCGGTGTGCACACCTTCCCAGCTGTCCTG | |
| | CAGTCTGACCTCTACACTCTGAGCAGCTCAGTGACTGTCCCCTCCAGCACCTGGCCC | |
| | AGCGAGACCGTCACCTGCAACGTTGCCCACCCGGCCAGCAGCACCAAGGTGGACAA | |
| | GAAAATTGTGCCCAGGGATTGTGGTTGTAAGCCTTGCATATGTACAGTCCCAGAAGTA | |
| | TCATCTGTCTTCATCTTCCCCCCAAAGCCCAAGGATGTGCTCACCATTACTCTGACTC | |
| | CTAAGGTCACGTGTGTTGTGGTAGACATCAGCAAGGATGATCCCGAGGTCCAGTTCA | |
| | GCTGGTTTGTAGATGATGTGGAGGTGCACACAGCTCAGACGCAACCCCGGGAGGAG | |
| | CAGTTCAACAGCACTTTCCGCTCAGTCAGTGAACTTCCCATCATGCACCAGGACTGG | |
| | CTCAATGGCAAGGAGTTCAAATGCAGGGTCAACAGTGCAGCTTTCCCTGCCCCCATC | |
| | GAGAAAACCATCTCCAAAACCAAAGGCAGACCGAAGGCTCCACAGGTGTACACCATT | |
| | CCACCTCCCAAGGAGCAGATGGCCAAGGATAAAGTCAGTCTGACCTGCATGATAACA | |
| | GACTTCTTCCCTGAAGACATTACTGTGGAGTGGCAGTGGAATGGGCAGCCAGCGGAG | |
| | AACTACAAGAACACTCAGCCCATCATGGACACAGATGGCTCTTACTTCGTCTACAGCA | |
| | AGCTCAATGTGCAGAAGAGCAACTGGGAGGCAGGAAATACTTTCACCTGCTCTGTGT | |
| | TACATGAGGGCCTGCACAACCACCATACTGAGAAGAGCCTCTCCCACTCTCCTGGTA | |
| | AA | |
| HCVR | CAGGCTTATCTACAGCAGTCTGGGGCTGAGCTGGTGAGGTCTGGGGCCTCAGTGAA | 22 |
| | GATGTCCTGCAAGGCTTCTGGCTACACATTTACCAGTTACAATATGCACTGGGTAAAG | |
| | CAGACACCTGGACAGGGCCTGGAATGGATTGGATATATTTATCCTGGAAATGGTGATA | |
| | TTAATTACAATCAGAAGTTTAAGGGCAAGGCCACATTGACTGCAGACACATCCTCCAA | |
| | CACAGCCTACATGCAGATCAGCAGCCTGACATCTGAAGACTCTGCGGTCTATTTCTGT | |
| | GCAAGAGGGGGGGTCTTTGACTACTGGGGCCAAGGCACCACTCTCATAGTCTCCTCA | |
| HC FR1 | CAGGCTTATCTACAGCAGTCTGGGGCTGAGCTGGTGAGGTCTGGGGCCTCAGTGAA | 23 |
| | GATGTCCTGCAAGGCTTCT | |
| HCDR1 | GGCTACACATTTACCAGTTACAAT | 24 |
| HC FR2 | ATGCACTGGGTAAAGCAGACACCTGGACAGGGCCTGGAATGGATTGGATAT | 25 |
| HCDR2 | ATTTATCCTGGAAATGGTGATATT | 26 |
| HC FR3 | AATTACAATCAGAAGTTTAAGGGCAAGGCCACATTGACTGCAGACACATCCTCCAACA | 27 |
| | CAGCCTACATGCAGATCAGCAGCCTGACATCTGAAGACTCTGCGGTCTATTTCTGT | |
| HCDR3 | GCAAGAGGGGGGGTCTTTGACTAC | 28 |
| HC FR4 | TGGGGCCAAGGCACCACTCTCATAGTCTCCTCA | 29 |
| HCCR | GCCAAAACGACACCCCCATCTGTCTATCCACTGGCCCCTGGATCTGCTGCCCAAACT | 30 |
| | AACTCCATGGTGACCCTGGGATGCCTGGTCAAGGGCTATTTCCCTGAGCCAGTGACA | |
| | GTGACCTGGAACTCTGGATCCCTGTCCAGCGGTGTGCACACCTTCCCAGCTGTCCTG | |
| | CAGTCTGACCTCTACACTCTGAGCAGCTCAGTGACTGTCCCCTCCAGCACCTGGCCC | |
| | AGCGAGACCGTCACCTGCAACGTTGCCCACCCGGCCAGCAGCACCAAGGTGGACAA | |
| | GAAAATTGTGCCCAGGGATTGTGGTTGTAAGCCTTGCATATGTACAGTCCCAGAAGTA | |
| | TCATCTGTCTTCATCTTCCCCCCAAAGCCCAAGGATGTGCTCACCATTACTCTGACTC | |
| | CTAAGGTCACGTGTGTTGTGGTAGACATCAGCAAGGATGATCCCGAGGTCCAGTTCA | |
| | GCTGGTTTGTAGATGATGTGGAGGTGCACACAGCTCAGACGCAACCCCGGGAGGAG | |
| | CAGTTCAACAGCACTTTCCGCTCAGTCAGTGAACTTCCCATCATGCACCAGGACTGG | |
| | CTCAATGGCAAGGAGTTCAAATGCAGGGTCAACAGTGCAGCTTTCCCTGCCCCCATC | |
| | GAGAAAACCATCTCCAAAACCAAAGGCAGACCGAAGGCTCCACAGGTGTACACCATT | |
| | CCACCTCCCAAGGAGCAGATGGCCAAGGATAAAGTCAGTCTGACCTGCATGATAACA | |
| | GACTTCTTCCCTGAAGACATTACTGTGGAGTGGCAGTGGAATGGGCAGCCAGCGGAG | |
| | AACTACAAGAACACTCAGCCCATCATGGACACAGATGGCTCTTACTTCGTCTACAGCA | |
| | AGCTCAATGTGCAGAAGAGCAACTGGGAGGCAGGAAATACTTTCACCTGCTCTGTGT | |
| | TACATGAGGGCCTGCACAACCACCATACTGAGAAGAGCCTCTCCCACTCTCCTGGTA | |
| | AA | |
| Light | CAAATTGTTCTCACCCAGTCTCCAGCAATCGTGTCTGCATCTCCAGGGGAGAAGGTC | 31 |
| chain | ACCATAACCTGCAGTGGCAGCTCAAGTGTAAATTACATGCACTGGTTCCAGCAGAAG | |
| | CCAGGCACTTCTCCCAAACTCTGGATTTATAGCACATCCAACCTGGCTTCTGGAGTCC | |
| | CTGGTCGCTTCAGTGGCAGTGGATCTGGGACCTCTTACTCTCTCACAATCAGCCGAA | |
| | TGGAGGCTGAAGATGCTGCCACTTATTACTGCCAACAAAGGACTAGTTACCCGTACAC | |
| | GTTCGGAGGGGGGACCAAGCTGGAAATAAAACGGGCTGATGCTGCACCAACTGTATC | |
| | CATCTTCCCACCATCCAGTGAGCAGTTAACATCTGGAGGTGCCTCAGTCGTGTGCTTC | |
| | TTGAACAACTTCTACCCCAAAGACATCAATGTCAAGTGGAAGATTGATGGCAGTGAAC | |
| | GACAAAATGGCGTCCTGAACAGTTGGACTGATCAGGACAGCAAAGACAGCACCTACA | |
| | GCATGAGCAGCACCCTCACGTTGACCAAGGACGAGTATGAACGACATAACAGCTATA | |
| | CCTGTGAGGCCACTCACAAGACATCAACTTCACCCATTGTCAAGAGCTTCAACAGGAA | |
| | TGAGTGT | |
| LCVR | CAAATTGTTCTCACCCAGTCTCCAGCAATCGTGTCTGCATCTCCAGGGGAGAAGGTC | 32 |
| | ACCATAACCTGCAGTGGCAGCTCAAGTGTAAATTACATGCACTGGTTCCAGCAGAAG | |
| | CCAGGCACTTCTCCCAAACTCTGGATTTATAGCACATCCAACCTGGCTTCTGGAGTCC | |
| | CTGGTCGCTTCAGTGGCAGTGGATCTGGGACCTCTTACTCTCTCACAATCAGCCGAA | |
| | TGGAGGCTGAAGATGCTGCCACTTATTACTGCCAACAAAGGACTAGTTACCCGTACAC | |
| | GTTCGGAGGGGGGACCAAGCTGGAAATAAAA | |
| LC FR1 | CAAATTGTTCTCACCCAGTCTCCAGCAATCGTGTCTGCATCTCCAGGGGAGAAGGTC | 33 |
| | ACCATAACCTGCAGTGGCAGC | |
| LCDR1 | TCAAGTGTAAATTAC | 34 |
| LC FR2 | ATGCACTGGTTCCAGCAGAAGCCAGGCACTTCTCCCAAACTCTGGATTTAT | 35 |
| LCDR2 | AGCACATCC | 36 |
| LC FR3 | AACCTGGCTTCTGGAGTCCCTGGTCGCTTCAGTGGCAGTGGATCTGGGACCTCTTAC | 37 |
| | TCTCTCACAATCAGCCGAATGGAGGCTGAAGATGCTGCCACTTATTACTGC | |
| LCDR3 | CAACAAAGGACTAGTTACCCGTACACG | 38 |
| LC FR4 | TTCGGAGGGGGGACCAAGCTGGAAATAAAA | 39 |
| LCCR | CGGGCTGATGCTGCACCAACTGTATCCATCTTCCCACCATCCAGTGAGCAGTTAACAT | 40 |
| | CTGGAGGTGCCTCAGTCGTGTGCTTCTTGAACAACTTCTACCCCAAAGACATCAATGT | |
| | CAAGTGGAAGATTGATGGCAGTGAACGACAAAATGGCGTCCTGAACAGTTGGACTGA | |
| | TCAGGACAGCAAAGACAGCACCTACAGCATGAGCAGCACCCTCACGTTGACCAAGGA | |
| | CGAGTATGAACGACATAACAGCTATACCTGTGAGGCCACTCACAAGACATCAACTTCA | |
| | CCCATTGTCAAGAGCTTCAACAGGAATGAGTGT | |

TABLE 4
Porcine IgG & IgA amino acid sequences

| | | SED IQ |
|---|---|---|
| Region | Amino acid sequence | NO |
| IgG Constant | RADAKPSVFIFPPSKEQLETQTVSVVCLLNSFFPREVNVKWKVDGVVQSSGILDSVT | 58 |
| light chain | EQDSKDSTYSLSSTLSLPTSQYLSHNLYSCEVTHKTLASPLVKSFNRNECEA | |
| IgG1a | APKTAPSVYPLAPCGRDTSGPNVALGCLASSYFPEPVTMTWNSGALTSGVHTFPSV | 59 |
| constant | LQPSGLYSLSSMVTVPASSLSSKSYTCNVNHPATTTKVDKRVGTKTKPPCPICPGCE | |
| heavy chain | VAGPSVFIFPPKPKDTLMISQTPEVTCVVVDVSKEHAEVQFSWYVDGVEVHTAETRP | |
| | KEEQFNSTYRVVSVLPIQHQDWLKGKEFKCKVNNVDLPAPITRTISKAIGQSREPQVY | |
| | TLPPPAEELSRSKVTVTCLVIGFYPPDIHVEWKSNGQPEPEGNYRTTPPQQDVDGTF | |
| | FLYSKLAVDKARWDHGETFECAVMHEALHNHYTQKSISKTQGK | |
| IgG1b | APKTAPSVYPLAPCGRDVSGPNVALGCLASSYFPEPVTVTWNSGALTSGVHTFPSV | 60 |
| constant | LQPSGLYSLSSMVTVPASSLSSKSYTCNVNHPATTTKVDKRVGIHQPQTCPICPGCE | |
| heavy chain | VAGPSVFIFPPKPKDTLMISQTPEVTCVVVDVSKEHAEVQFSWYVDGVEVHTAETRP | |
| | KEEQFNSTYRVVSVLPIQHQDWLKGKEFKCKVNNVDLPAPITRTISKAIGQSREPQVY | |
| | TLPPPAEELSRSKVTLTCLVIGFYPPDIHVEWKSNGQPEPENTYRTTPPQQDVDGTF | |
| | FLYSKLAVDKARWDHGDKFECAVMHEALHNHYTQKSISKTQGK | |
| IgG2a | APKTAPSVYPLAPCSRDTSGPNVALGCLASSYFPEPVTVTWNSGALSSGVHTFPSV | 61 |
| constant | LQPSGLYSLSSMVTVPASSLSSKSYTCNVNHPATTTKVDKRVGTKTKPPCPICPACE | |
| heavy chain | SPGPSVFIFPPKPKDTLMISRTPQVTCVVVDVSQENPEVQFSWYVDGVEVHTAQTR | |
| | PKEEQFNSTYRVVSVLPIQHQDWLNGKEFKCKVNNKDLPAPITRIISKAKGQTREPQ | |
| | VYTLPPHAEELSRSKVSITCLVIGFYPPDIDVEWQRNGQPEPEGNYRTTPPQQDVDG | |
| | TYFLYSKFSVDKASWQGGGIFQCAVMHEALHNHYTQKSISKTPGK | |
| IgG2b | APKTAPLVYPLAPCGRDTSGPNVALGCLASSYFPEPVTVTWNSGALTSGVHTFPSVL | 62 |
| constant | QPSGLYSLSSMVTVPASSLSSKSYTCNVNHPATTTKVDKRVGTKTKPPCPICPACES | |
| heavy chain | PGPSVFIFPPKPKDTLMISRTPQVTCVVVDVSQENPEVQFSWYVDGVEVHTAQTRP | |
| | KEEQFNSTYRVVSVLPIQHQDWLNGKEFKCKVNNKDLPAPITRIISKAKGQTREPQV | |
| | YTLPPHAEELSRSKVSITCLVIGFYPPDIDVEWQRNGQPEPEGNYRTTPPQQDVDGT | |
| | YFLYSKFSVDKASWQGGGIFQCAVMHEALHNHYTQKSISKTPGK | |
| IgG3 | AYNTAPSVYPLAPCGRDVSDHNVALGCLVSSYFPEPVTVTWNSGALSRVVHTFPSV | 63 |
| constant | LQPSGLYSLSSMVIVAASSLSTLSYTCNVYHPATNTKVDKRVDIEPPTPICPEICSCPA | |
| heavy chain | AEVLGAPSVFLFPPKPKDILMISRTPKVTCVVVDVSQEEAEVQFSWYVDGVQLYTAQ | |
| | TRPMEEQFNSTYRVVSVLPIQHQDWLKGKEFKCKVNNKDLLSPITRTISKATGPSRV | |
| | PQVYTLPPAWEELSKSKVSITCLVTGFYPPDIDVEWQSNGQQEPEGNYRTTPPQQD | |
| | VDGTYFLYSKLAVDKVRWQRGDLFQCAVMHEALHNHYTQKSISKTQGK | |
| IgG4a | TFPSVLQPSGLYSLSSMVTVPASSLSSKSYTCNVNHPATTTKVDKRVGTKTKPPCPI | 64 |
| constant | CPACEGPGPSAFIFPPKPKDTLMISRTPKVTCVVVDVSQENPEVQFSWYVDGVEVH | |
| heavy chain | TAQTRPKEEQFNSTYRVVSVLPIQHQDWLNGKEFKCKVNNKDLPAPITRIISKAKGQT | |
| | REPQVYTLPPPTEELSRSKVTLTCLVTGFYPPDIDVEWQRNGQPEPEGNYRTTPPQ | |
| | QDVDGTYFLYSKLAVDKASWQRGDTFQCAVMHEALHNHYTQKSIFKTPGK | |
| IgG4b | APKTAPSVYPLAPCGRDVSGPNVALGCLASSYFPEPVTVTWNSGALTSGVHTFPSV | 65 |
| constant | LQPSGLYSLSSMVTVPASSLSSKSYTCNVNHPATTTKVDKRVGIHQPQTCPICPACE | |
| heavy chain | GPGPSAFIFPPKPKDTLMISRTPKVTCVVVDVSQENPEVQFSWYVDGVEVHTAQTR | |
| | PKEEQFNSTYRVVSVLLIQHQDWLNGKEFKCKVNNKDLPAPITRIISKAKGQTREPQV | |
| | YTLPPPTEELSRSKVTLTCLVTGFYPPDIDVEWQRNGQPEPEGNYRTTPPQQDVDG | |
| | TYFLYSKLAVDKASWQRGDTFQCAVMHEALHNHYT | |
| IgG5a | APKTAPSVYPLAPCSRDTSGPNVALGCLVSSYFPEPVTVTWNSGALTSGVHTFPSVL | 66 |
| constant | QPSGLYSLSSMVTVPAHSLSSKRYTCNVNHPATKTKVDLCVGRPCPICPGCEVAGP | |
| heavy chain | SVFIFPPKPKDILMISRTPEVTCVVVDVSKEHAEVQFSWYVDGEEVHTAETRPKEEQ | |
| | FNSTYRVVSVLPIQHEDWLKGKEFECKVNNEDLPGPITRTISKAKGVVRSPEVYTLPP | |
| | PAEELSKSIVTLTCLVKSIFPXFIHVEWKINGKPEPENAYRTTPPQEDEDRTYFLYSKL | |
| | AVDKARWDHGETFECAVMHEALHNHYTQKSISKTQGK | |
| IgG5b | AYNTAPSVYPLAPCGRDVSDHNVALGCLVSSYFPEPVTVTWNWGAQTSGVHTFPS | 67 |
| constant | VLQPSGLYSLSSTVTVPAHSLSSKCFTCNVNHPATTTKVDLCVGKKTKPRCPICPGC | |
| heavy chain | EVAGPSVFIFPPKPKDILMISRTPEVTCVVVDVSKEHAEVQFSWYVDGEEVHTAETR | |
| | PKEEQFNSTYRVVSVLPIQHEDWLKGKEFECKVNNEDLPGPITRTISKAKGVVRSPE | |
| | VYTLPPPAEELSKSIVTLTCLVKSFFPPFIHVEWKINGKPEPENAYRTTPPQEDEDGT | |
| | YFLYSKFSVEKFRWHSGGIHCAVMHEALHNHYT | |
| IgG6a | APKTAPSVYPLAPCGRDTSGPNVALGCLASSYFPEPVTLTWNSGALTSGVHTFPSVL | 68 |
| constant | QPSGLYSLSSMVTVPASSLSSKSYTCNVNHPATTTKVDLCVGRPCPICPACEGPGP | |
| heavy chain | SVFIFPPKPKDTLMISRTPQVTCVVVDVSQENPEVQFSWYVDGVEVHTAQTRPKEA | |
| | QFNSTYRVVSVLPIQHEDWLKGKEFECKVNNKDLPAPITRIISKAKGPSREPQVYTLS | |
| | PSAEELSRSKVSITCLVTGFYPPDIDVEWKSNGQPEPEGNYRTTPPQQDVDGTYFLY | |
| | SKLAVDKASWQRGDPFQCAVMHEALHNHYT | |
| IgG6b | APKTAPSVYPLAPCGRDTSGPNVALGCLASSYFPEPVTVTWNSGALTSGVHTFPSV | 69 |
| constant | LQPSGLYSLSSTVTVPARSSSRKCFTCNVNHPATTTKVDLCVGRPCPICPACEGNGP | |
| heavy chain | SVFIFPPKPKDTLMISRTPEVTCVVVDVSQENPEVQFSWYVDGEEVHTAETRPKEEQ | |
| | FNSTYRVVSVLPIQHQDWLKGKEFECKVNNKDLPAPITRIISKAKGPSREPQVYTLSP | |
| | SAEELSRSKVSITCLVTGFYPPDIDVEWKSNGQPEPEGNYRSTPPQEDEDGTYFLYS | |
| | KLAVDKARLQSGGIHCAVMHEALHNHYTQKSISKT | |
| IgA constant | RADAKPSVFIFPPSKEQLETQTVSVVCLLNSFFPREVNVKWKVDGVVQSSGILDSVT | 58 |
| light chain | EQDSKDSTYSLSSTLSLPTSQYLSHNLYSCEVTHKTLASPLVKSFNRNECEA | |
| IgA Constant | SETSPKIFPLTLGSSEPAGYVVIACLVRDFFPSEPLTVTWSPSREGVIVRNFPPAQAG | 70 |
| heavy chain | GLYTMSSQLTLPVEQCPADQILKCQVQHLSKSSQSVNVPCKVLPSDPCPQCCKPSL | |
| | SLQPPALADLLLGSNASLTCTLSGLKKSEGVSFTWQPSGGKDAVQASPTRDSCGCY | |
| | SVSSILPGCADPWNKGETFSCTAAHSELKSALTATITKPKVNTFRPQVHLLPPPSEEL | |
| | ALNELVTLTCLVRGFSPKDVLVRWLQGGQELPRDKYLVWESLPEPGQAIPTYAVTSV | |
| | LRVDAEDWKQGDTFSCMVGHEALPLAFTQKTIDRLAGKPTHVNVSVVMAEAEGICY | |

TABLE 5
Porcine IgG & IgA nucleic acid sequences

| | | SED IQ |
|---|---|---|
| Region | nucleic acid sequence | NO |
| IgG Constant | CGGGCTGATGCCAAGCCATCCGTCTTCATCTTCCCGCCATCGAAGGAGCAGTT | 71 |
| light chain | AGAGACCCAAACTGTCTCTGTGGTGTGCTTGCTCAATAGCTTCTTCCCCAGAGA | |
| | AGTCAATGTCAAGTGGAAAGTGGATGGGGTGGTCCAAAGCAGTGGCATCTTGG | |
| | ATAGTGTCACAGAGCAGGACAGCAAGGACAGCACCTACAGCCTCAGCAGCAC | |
| | CCTCTCGCTGCCCACGTCACAGTACCTAAGTCATAATTTATATTCCTGTGAGGT | |
| | CACCCACAAGACCCTGGCCTCCCCTCTGGTCAAAAGCTTCAACAGGAACGAGT | |
| | GTGAGGCT | |
| IgG1a | GCCCCCAAGACGGCCCCATCGGTCTACCCTCTGGCCCCCTGCGGCAGGGACA | 72 |
| constant | CGTCTGGCCCTAACGTGGCCTTGGGCTGCCTGGCCTCAAGCTACTTCCCCGA | |
| heavy chain | GCCAGTGACCATGACCTGGAACTCGGGCGCCCTGACCAGTGGCGTGCATACC | |
| | TTCCCATCCGTCCTGCAGCCGTCAGGGCTCTACTCCCTCAGCAGCATGGTGAC | |
| | CGTGCCGGCCAGCAGCCTGTCCAGCAAGAGCTACACCTGCAATGTCAACCAC | |
| | CCGGCCACCACCACCAAGGTGGACAAGCGTGTTGGAACAAAGACCAAACCAC | |
| | CATGTCCCATATGCCCAGGCTGTGAAGTGGCCGGGCCCTCGGTCTTCATCTTC | |
| | CCTCCAAAACCCAAGGACACCCTCATGATCTCCCAGACCCCCGAGGTCACGTG | |
| | CGTGGTGGTGGACGTCAGCAAGGAGCACGCCGAGGTCCAGTTCTCCTGGTAC | |
| | GTGGACGGCGTAGAGGTGCACACGGCCGAGACGAGACCAAAGGAGGAGCAG | |
| | TTCAACAGCACCTACCGTGTGGTCAGCGTCCTGCCCATCCAGCACCAGGACTG | |
| | GCTGAAGGGGAAGGAGTTCAAGTGCAAGGTCAACAACGTAGACCTCCCAGCC | |
| | CCCATCACGAGGACCATCTCCAAGGCTATAGGGCAGAGCCGGGAGCCGCAGG | |
| | TGTACACCCTGCCCCCACCCGCCGAGGAGCTGTCCAGGAGCAAAGTCACCGT | |
| | AACCTGCCTGGTCATTGGCTTCTACCCACCTGACATCCATGTTGAGTGGAAGA | |
| | GCAACGGACAGCCGGAGCCAGAGGGCAATTACCGCACCACCCCGCCCCAGCA | |
| | GGACGTGGACGGGACCTTCTTCCTGTACAGCAAGCTCGCGGTGGACAAGGCA | |
| | AGATGGGACCATGGAGAAACATTTGAGTGTGCGGTGATGCACGAGGCTCTGCA | |
| | CAACCACTACACCCAGAAGTCCATCTCCAAGACTCAGGGTAAATGAGCCACCC | |
| | GCTGCACCCCACGTGCTCTCGGGTCCCAAGAGCTCGCCTAAGCCCCAGCGCT | |
| | GTGTACATACGTCCCGGGCCAGCATGAAATAAA | |
| IgG1b | GCCCCCAAGACGGCCCCATCGGTCTACCCTCTGGCCCCCTGCGGCAGGGACG | 73 |
| constant | TGTCTGGCCCTAACGTGGCCTTGGGCTGCCTGGCCTCAAGCTACTTCCCCGAG | |
| heavy chain | CCAGTGACCGTGACCTGGAACTCGGGCGCCCTGACCAGTGGCGTGCACACCT | |
| | TCCCATCCGTCCTGCAGCCGTCAGGGCTCTACTCCCTCAGCAGCATGGTGACC | |
| | GTGCCGGCCAGCAGCCTGTCCAGCAAGAGCTACACCTGCAATGTCAACCACC | |
| | CGGCCACCACCACCAAGGTGGACAAGCGTGTTGGAATACACCAGCCGCAAAC | |
| | ATGTCCCATATGCCCAGGCTGTGAAGTGGCCGGGCCCTCGGTCTTCATCTTCC | |
| | CTCCAAAACCCAAGGACACCCTCATGATCTCCCAGACCCCCGAGGTCACGTGC | |
| | GTGGTGGTGGACGTCAGCAAGGAGCACGCCGAGGTCCAGTTCTCCTGGTACG | |
| | TGGACGGCGTAGAGGTGCACACGGCCGAGACGAGACCAAAGGAGGAGCAGTT | |
| | CAACAGCACCTACCGTGTGGTCAGCGTCCTGCCCATCCAGCACCAGGACTGG | |
| | CTGAAGGGGAAGGAGTTCAAGTGCAAGGTCAACAACGTAGACCTCCCAGCCC | |
| | CCATCACGAGGACCATCTCCAAGGCTATAGGGCAGAGCCGGGAGCCGCAGGT | |
| | GTACACCCTGCCCCCACCCGCCGAGGAGCTGTCCAGGAGCAAAGTCACGCTA | |
| | ACCTGCCTGGTCATTGGCTTCTACCCACCTGACATCCATGTTGAGTGGAAGAG | |
| | CAACGGACAGCCGGAGCCAGAGAACACATACCGCACCACCCCGCCCCAGCAG | |
| | GACGTGGACGGGACCTTCTTCCTGTACAGCAAACTCGCGGTGGACAAGGCAA | |
| | GATGGGACCATGGAGACAAATTTGAGTGTGCGGTGATGCACGAGGCTCTGCA | |
| | CAACCACTACACCCAGAAGTCCATCTCCAAGACTCAGGGTAAATGAGCCACCC | |
| | GCTGCACCCCACGTGCTCTCGGGTCCCGCGAGCTCGCCTGAGCCCCAGCGCT | |
| | GTGTACATACGTCCCGGGCCAGCATGAAATAAA | |
| IgG2a | GCCCCCAAGACGGCCCCATCGGTCTACCCTCTGGCCCCCTGCAGCAGGGACA | 74 |
| constant | CGTCTGGCCCTAACGTGGCCTTGGGCTGCCTGGCCTCAAGCTACTTCCCCGA | |
| heavy chain | GCCAGTGACCGTGACCTGGAACTCGGGCGCCCTGTCCAGTGGCGTGCATACC | |
| | TTCCCATCCGTCCTGCAGCCGTCAGGGCTCTACTCCCTCAGCAGCATGGTGAC | |
| | CGTGCCGGCCAGCAGCCTGTCCAGCAAGAGCTACACCTGCAATGTCAACCAC | |
| | CCGGCCACCACCACCAAGGTGGACAAGCGTGTTGGAACAAAGACCAAACCAC | |
| | CATGTCCCATATGCCCAGCCTGTGAATCACCAGGGCCCTCGGTCTTCATCTTC | |
| | CCTCCAAAACCCAAGGACACCCTCATGATCTCCCGGACACCCCAGGTCACGTG | |
| | CGTGGTGGTTGATGTGAGCCAGGAGAACCCGGAGGTCCAGTTCTCCTGGTAC | |
| | GTGGACGGCGTAGAGGTGCACACGGCCCAGACGAGGCCAAAGGAGGAGCAG | |
| | TTCAACAGCACCTACCGCGTGGTCAGCGTCCTACCCATCCAGCACCAGGACTG | |
| | GCTGAACGGGAAGGAGTTCAAGTGCAAGGTCAACAACAAAGACCTCCCAGCC | |
| | CCCATCACAAGGATCATCTCCAAGGCCAAAGGGCAGACCCGGGAGCCGCAGG | |
| | TGTACACCCTGCCCCCACACGCCGAGGAGCTGTCCAGGAGCAAAGTCAGCAT | |
| | AACCTGCCTGGTCATTGGCTTCTACCCACCTGACATCGATGTCGAGTGGCAAA | |
| | GAAACGGACAGCCGGAGCCAGAGGGCAATTACCGCACCACCCCGCCCCAGCA | |
| | GGACGTGGACGGGACCTACTTCCTGTACAGCAAGTTCTCGGTGGACAAGGCC | |
| | AGCTGGCAGGGTGGAGGCATATTCCAGTGTGCGGTGATGCACGAGGCTCTGC | |
| | ACAACCACTACACCCAGAAGTCTATCTCCAAGACTCCGGGTAAATGAGCCACT | |
| | CGCTGCACCCCTCAAGCTCTTGGGTCCCAAGAGCTCACCTGAGCCCCAGCCCT | |
| | GTGTACATATCGCCCGGGCCAGCATGAAATAAA | |
| IgG2b | GCCCCCAAGACGGCCCCATTGGTCTACCCTCTGGCCCCCTGCGGCAGGGACA | 75 |
| constant | CGTCTGGCCCTAACGTGGCCTTGGGCTGCCTGGCCTCAAGCTACTTCCCCGA | |
| heavy chain | GCCAGTGACCGTGACCTGGAACTCGGGCGCCCTGACCAGTGGCGTGCATACC | |
| | TTCCCATCCGTCCTGCAGCCGTCAGGGCTCTACTCCCTCAGCAGCATGGTGAC | |
| | CGTGCCGGCCAGCAGCCTGTCCAGCAAGAGCTACACCTGCAATGTCAACCAC | |
| | CCGGCCACCACCACCAAGGTGGACAAGCGTGTTGGAACAAAGACCAAACCAC | |
| | CATGTCCCATATGCCCAGCCTGTGAATCGCCAGGGCCCTCGGTCTTCATCTTC | |
| | CCTCCAAAACCCAAGGACACCCTCATGATCTCCCGGACACCCCAGGTCACGTG | |
| | CGTGGTAGTTGATGTGAGCCAGGAGAACCCGGAGGTCCAGTTCTCCTGGTAC | |
| | GTGGACGGCGTAGAGGTGCACACGGCCCAGACGAGGCCAAAGGAGGAGCAG | |
| | TTCAACAGCACCTACCGCGTGGTCAGCGTCCTGCCCATCCAGCACCAGGACTG | |
| | GCTGAACGGGAAGGAGTTCAAGTGCAAGGTCAACAACAAAGACCTCCCAGCC | |
| | CCCATCACAAGGATCATCTCCAAGGCCAAAGGGCAGACCCGGGAGCCGCAGG | |
| | TGTACACCCTGCCCCCACACGCCGAGGAGCTGTCCAGGAGCAAAGTCAGCAT | |
| | AACCTGCCTGGTCATTGGCTTCTACCCACCTGACATCGATGTCGAGTGGCAAA | |
| | GAAACGGACAGCCGGAGCCAGAGGGCAATTACCGCACCACCCCGCCCCAGCA | |
| | GGACGTGGACGGGACCTACTTCCTGTACAGCAAGTTCTCGGTGGACAAGGCC | |
| | AGCTGGCAGGGTGGAGGCATATTCCAGTGTGCGGTGATGCACGAGGCTCTGC | |
| | ACAACCACTACACCCAGAAGTCTATCTCCAAGACTCCGGGTAAATGAGCCACT | |
| | CGCTGCACCCCTCATGCTCTTGGGTCCCAAGAGCTCACCTGAGCCCCAGCGCT | |
| | GTGTACATACGTCCCGGGCCAGCATGAAATAAA | |
| IgG3 constant | GCCTACAACACAGCTCCATCGGTCTACCCTCTGGCCCCCTGTGGCAGGGACGT | 76 |
| heavy chain | GTCTGATCATAACGTGGCCTTGGGCTGCCTTGTCTCAAGCTACTTCCCCGAGC | |
| | CAGTGACCGTGACCTGGAACTCGGGTGCCCTGTCCAGAGTCGTGCATACCTTC | |
| | CCATCCGTCCTGCAGCCGTCAGGGCTCTACTCCCTCAGCAGCATGGTGATCGT | |
| | GGCGGCCAGCAGCCTGTCCACCCTGAGCTACACGTGCAACGTCTACCACCCG | |
| | GCCACCAACACCAAGGTGGACAAGCGTGTTGGTGAGCGCCTGCGCTGGGGGC | |
| | GGGAGTGCACGTCAAGACAGGCCGGGGTGAGCCCCCTGCCCGCAGGGACCA | |
| | GTTCCCAGGATGGAGAGTCCCACCCAGGGTGTCCCCCTCACCTGCAGGCCCA | |
| | GGCTCAGGGAGGGGTCAACTCGGCACTTCGGAGAGGCCAGGGTGGGCACAG | |
| | GCGGGACCCCTCCCCCGGCCCTGGGCCCCCAGCAGCACGTGATGCTCTGGC | |
| | ATGTACCAAGACCTGACCCTGACCTGAGCCCAGCCCTGGGCCCTCCCCACCC | |
| | CCAGTGACCCTGTGTGTTCTCTCTGCAGACATCGAACCCCCCACACCCATCTG | |
| | TCCCGAAATTTGCTCATGCCCAGGTGAGTCAGTCGGGCGTGACCCTCCTCCTG | |
| | AAGAGGTGGCCCGAGCTGGGTCTCAACACGAGGGTGCACGGGTGCCCAGGA | |
| | GCACCCCAGGACAGGTGCTGACCCCACACTGTGTCTCCTCCACCAGCTGCAG | |
| | AGGTCCTGGGAGCACCGTCGGTCTTCCTCTTCCCTCCAAAACCCAAGGACATC | |
| | CTCATGATCTCCCGGACACCCAAGGTCACGTGCGTGGTGGTGGACGTGAGCC | |
| | AGGAGGAGGCTGAAGTCCAGTTCTCCTGGTACGTGGACGGCGTACAGTTGTA | |
| | CACGGCCCAGACGAGGCCAATGGAGGAGCAGTTCAACAGCACCTACCGCGTG | |
| | GTCAGCGTCCTGCCCATCCAGCACCAGGACTGGCTGAAGGGGAAGGAGTTCA | |
| | AGTGCAAGGTCAACAACAAAGACCTCCTTTCCCCCATCACGAGGACCATCTCC | |
| | AAGGCTACAGGTGCATGGGGTGAGAGGCAGGGAGGGTCCCGTGGGGCCACC | |
| | TGGGGGGACCATCATGCTAACAGAGGTGTCTGGCCTCACAGGGCCGAGCCGG | |
| | GTGCCGCAGGTGTACACCCTGCCCCCAGCCTGGGAAGAGCTGTCCAAGAGCA | |
| | AAGTCAGCATAACCTGCCTGGTCACTGGCTTCTACCCACCTGACATCGATGTC | |
| | GAGTGGCAGAGCAACGGACAACAAGAGCCAGAGGGCAATTACCGCACCACCC | |
| | CGCCCCAGCAGGACGTGGATGGGACCTACTTCCTGTACAGCAAGCTCGCGGT | |
| | GGACAAGGTCAGGTGGCAGCGTGGAGACCTATTCCAGTGTGCGGTGATGCAC | |
| | GAGGCTCTGCACAACCACTACACCCAGAAGTCCATCTCCAAGACTCAGGGTAA | |
| | ATGA | |
| IgG4a | ATACCTTCCCATCCGTCCTGCAGCCGTCAGGGCTCTACTCCCTCAGCAGCATG | 77 |
| constant | GTGACCGTGCCGGCCAGCAGCCTGTCCAGCAAGAGCTACACCTGCAATGTCA | |
| heavy chain | ACCACCCGGCCACCACCACCAAGGTGGACAAGCGTGTTGGAACAAAGACCAA | |
| | ACCACCATGTCCCATATGCCCAGCCTGTGAAGGGCCCGGGCCCTCGGCCTTC | |
| | ATCTTCCCTCCAAAACCCAAGGACACCCTCATGATCTCCCGGACCCCCAAGGT | |
| | CACGTGCGTGGTGGTAGATGTGAGCCAGGAGAACCCGGAGGTCCAGTTCTCC | |
| | TGGTACGTGGACGGCGTAGAGGTGCACACGGCCCAGACGAGGCCAAAGGAG | |
| | GAGCAGTTCAACAGCACCTACCGCGTGGTCAGCGTCCTGCCCATCCAGCACC | |
| | AGGACTGGCTGAACGGGAAGGAGTTCAAGTGCAAGGTCAACAACAAAGACCTC | |
| | CCAGCCCCCATCACAAGGATCATCTCCAAGGCCAAAGGGCAGACCCGGGAGC | |
| | CGCAGGTGTACACCCTGCCCCCACCCACCGAGGAGCTGTCCAGGAGCAAAGT | |
| | CACGCTAACCTGCCTGGTCACTGGCTTCTACCCACCTGACATCGATGTCGAGT | |
| | GGCAAAGAAACGGACAGCCGGAGCCAGAGGGCAATTACCGCACCACCCCGCC | |
| | CCAGCAGGACGTGGACGGGACCTACTTCCTGTACAGCAAGCTCGCGGTGGAC | |
| | AAGGCCAGCTGGCAGCGTGGAGACACATTCCAGTGTGCGGTGATGCACGAGG | |
| | CTCTGCACAACCACTACACCCAGAAGTCCATCTTCAAGACTCCGGGTAAATGA | |
| | GCCACTTGCTGCACCCCACGTGCTCTCGGGTCCCACGAGCTCGCCTGAGCCC | |
| | CAGCCCTGTGTACATACGTCCCGGGCCAGCATGAAATAAA | |
| IgG4b | GCCCCCAAGACGGCCCCATCGGTCTACCCTCTGGCCCCCTGCGGCAGGGACG | 78 |
| constant | TGTCTGGCCCTAACGTGGCCTTGGGCTGCCTGGCCTCAAGCTACTTCCCCGAG | |
| heavy chain | CCAGTGACCGTGACCTGGAACTCGGGCGCCCTGACCAGTGGCGTGCACACCT | |
| | TCCCATCCGTCCTGCAGCCGTCAGGGCTCTACTCCCTCAGCAGCATGGTGACC | |
| | GTGCCGGCCAGCAGCCTGTCCAGCAAGAGCTACACCTGCAATGTCAACCACC | |
| | CGGCCACCACCACCAAGGTGGACAAGCGTGTTGGTGAGCGCCTGCGCTGGG | |
| | GGCGGGAGTGCACGTCGAGACAGGCCGGGGTGAGCCCCCTGCCCGCAGGGA | |
| | CCAGTTCCCAGGATGGAGAGTCCCGCCCAGGGTGTCAGGGCCTCTCACCTCT | |
| | GTCCCCTTCACCTGCAGGCCCAGGCTCAGGGAGGGGTCAACTCGGCACTTCG | |
| | GAGAGGCCAGGGTGGGCACAGGCGGGACCCCTCCCCCGGCCCTGGGCCCCC | |
| | AGCAGCACGAGCTGCTCTGGGATGTACCAAGACCTGACCCTGACCTGAGCCC | |
| | AGCCCTGGGCCCTCCCCACCCCCAGTGACTCTGAGTGTTCTCTCCTCAGGAAT | |
| | ACACCAGCCGCAAACATGTCCCATATGCCCAGGTAAGAAACTCGGGCCTCGCC | |
| | CTCCTCCCCAAGGAGGTAGCCTGAGCCCAAGTTCGGCCCGAGGGGGACAGGT | |
| | GCCCAGGAGGACCCCAGGCCACGAGCTGACCCGCACTCTGTCTCCCCCACCA | |
| | GCCTGTGAAGGGCCCGGGCCCTCGGCCTTCATCTTCCCTCCAAAACCCAAGG | |
| | ACACCCTCATGATCTCCCGGACCCCCAAGGTCACGTGCGTGGTGGTTGATGTG | |
| | AGCCAGGAGAACCCGGAGGTCCAGTTCTCCTGGTACGTGGACGGCGTAGAGG | |
| | TGCACACGGCCCAGACGAGGCCAAAGGAGGAGCAGTTCAACAGCACCTACCG | |
| | CGTGGTCAGCGTCCTGCTCATCCAGCACCAGGACTGGCTGAACGGGAAGGAG | |
| | TTCAAGTGCAAGGTCAACAACAAAGACCTCCCAGCCCCCATCACAAGGATCAT | |
| | CTCCAAGGCCAAAGGTGGGCAAGGGGGGCAGGCGGAGAGGGACCTGTGGGG | |
| | CCACCTGGGGGGACCATCATGCTAACTGAGGTGCCTGGCCTCACAGGGCAGA | |
| | CCCGGGAGCCGCAGGTGTACACCCTGCCCCCACCCACCGAGGAGCTGTCCAG | |
| | GAGCAAAGTCACGCTAACCTGCCTGGTCACTGGCTTCTACCCACCTGACATCG | |
| | ATGTCGAGTGGCAAAGAAACGGACAGCCGGAGCCAGAGGGCAATTACCGCAC | |
| | CACCCCGCCCCAGCAGGACGTGGACGGGACCTACTTCCTGTACAGCAAGCTC | |
| | GCGGTGGACAAGGCCAGCTGGCAGCGTGGAGACACATTCCAGTGTGCGGTGA | |
| | TGCACGAGGCTCTGCACAACCACTACACCC | |
| IgG5a | GCCCCCAAGACGGCCCCATCGGTCTACCCTCTGGCCCCCTGCAGCAGGGACA | 79 |
| constant | CGTCTGGCCCTAACGTGGCCTTGGGCTGCCTGGTCTCAAGCTACTTCCCCGAG | |
| heavy chain | CCAGTGACCGTGACCTGGAACTCGGGCGCCCTGACCAGTGGCGTGCACACCT | |
| | TCCCATCCGTCCTGCAGCCGTCAGGGCTCTACTCCCTCAGCAGCATGGTGACC | |
| | GTGCCGGCCCACAGCTTGTCCAGCAAGCGCTATACGTGCAATGTCAACCACCC | |
| | AGCCACCAAAACCAAGGTGGACCTGTGTGTTGGTGAGTGCCTGGGCTGGGGG | |
| | CGGGTGTGCAGACGGGCTGAGGTCAGCCTTCCTGCCGAGAGACCACCCCACC | |
| | AGGGTGTCAGGGCCGCTCACCTCTGTCCCCTTCACCTGCAGGCCCAGGCTCA | |
| | GGGAGGGGTCAACTCGGCACTTCGGAGAGGCCAGGGTGGGCACAGGCGGGA | |
| | CCCCTCCCCCGGCCCTGGGCCCCCAGCAGCACGAGCTGCTCTGGGATGTACC | |
| | AAGACCTGACCCTGACCTGAGCCCAGCCCTGGACCCTCCCCACCCCCAGTGA | |
| | CTCTGTGTGTTCTCTCCTCAGGACGACCATGTCCCATATGCCCAGGTAAGAAAC | |
| | TCGGGCCTCGCCCTCCTCCCCAAGGAGGTAGCCTGAGCCCAGGTTCGGCCCG | |
| | AGGGGGGACAGGTGCCCAGGAGGACCCCAGGCCACGAGCTGACCCGCACTC | |
| | TGTCTCCCCCACCAGGCTGTGAAGTGGCCGGGCCCTCGGTCTTCATCTTCCCT | |
| | CCAAAACCCAAGGACATCCTCATGATCTCCCGGACCCCCGAGGTCACGTGCGT | |
| | GGTGGTGGACGTCAGCAAGGAGCACGCCGAGGTCCAGTTCTCCTGGTACGTG | |
| | GACGGCGAAGAGGTGCACACGGCCGAGACGAGGCCAAAGGAGGAGCAGTTC | |
| | AACAGCACCTACCGCGTGGTCAGCGTCCTGCCCATCCAGCACGAGGACTGGC | |
| | TGAAGGGGAAGGAGTTCGAGTGCAAGGTCAACAACGAAGACCTCCCAGGCCC | |
| | CATCACGAGGACCATCTCCAAGGCCAAAGGTGGGCAAGGTGGGCAGGCGGAG | |
| | AGGGTCCCGTGCGGCCACCTGGGGGGACCATCATGCTAACTGAGGTGCCTGG | |
| | CTTCACAGGGGTGGTACGGAGCCCGGAGGTGTACACCCTGCCCCCACCCGCC | |
| | GAGGAGCTGTCCAAGAGCATAGTCACGCTAACCTGCCTGGTCAAAAGCATCTT | |
| | CCCGNCTTTCATCCATGTTGAGTGGAAAATCAACGGAAAACCAGAGCCAGAGA | |
| | ACGCATATCGCACCACCCCGCCTCAGGAGGACGAGGACAGGACCTACTTCCT | |
| | GTACAGCAAGCTCGCGGTGGACAAGGCAAGATGGGACCATGGAGAAACATTT | |
| | GAGTGTGCGGTGATGCACGAGGCTCTGCACAACCACTACACCCAGAAGTCCAT | |
| | CTCCAAGACTCAGGGTAAATGA | |
| IgG5b | GCCTACAACACAGCTCCATCGGTCTACCCTCTGGCCCCCTGTGGCAGGGACGT | 80 |
| constant | GTCTGATCATAACGTGGCCTTGGGCTGCCTGGTCTCAAGCTACTTCCCCGAGC | |
| heavy chain | CAGTGACCGTGACCTGGAACTGGGGCGCCCAGACCAGTGGCGTGCACACCTT | |
| | CCCATCCGTCCTGCAGCCGTCAGGGCTCTACTCCCTCAGCAGCACGGTGACC | |
| | GTGCCGGCCCACAGCTTGTCCAGCAAGTGCTTCACGTGCAATGTCAACCACCC | |
| | GGCCACCACCACCAAGGTGGACCTGTGTGTTGGTGAGTGCCTGGACTGGGGG | |
| | CGGGTGTGCAGACGGGCTGAGGTCAGCCTTCCTGCCGAGAGACCACCCCCCC | |
| | AGGGTGTCAGGGCCGCTCACCTCTGTCCCCCTCACCTGTAGGCCCAGGCTCA | |
| | GGGAGGGGTCAACTCGGCACTTCGGAGAGGCCAGGGTGGGCACAGGCGGGA | |
| | CCCCTCCCCCGGCCCTGGGCCCCCAGCAGCACGAGCTGCTCTGGGATGTACC | |
| | AAGACCTGACCCTGACCTGAGCCCAGCCCTGGGCCCTCCCCACCCCCAGTGA | |
| | CTCTGTGTGTTCTCTCCTCAGGAAAAAAGACCAAGCCTCGATGTCCCATATGCC | |
| | CAGGTAAGAAACTCGGGCCTCGCCCTCCTCCCCAAGGAGGTAGCCTGAGCCC | |
| | AGGTTCGGCCCGAGGGGGGACAGGTGCCCAGGAGGACCCCAGGCCACGAGC | |
| | TGACCCGCACTCTGTCTCCCCCACCAGGCTGTGAAGTGGCCGGGCCCTCGGT | |
| | CTTCATCTTCCCTCCAAAACCCAAGGACATCCTCATGATCTCCCGGACCCCCG | |
| | AGGTCACGTGCGTGGTGGTGGACGTCAGCAAGGAGCACGCCGAGGTCCAGTT | |
| | CTCCTGGTACGTGGACGGCGAAGAGGTGCACACGGCCGAGACGAGACCAAAG | |
| | GAGGAGCAGTTCAACAGCACTTACCGCGTGGTCAGCGTCCTGCCCATCCAGC | |
| | ACGAGGACTGGCTGAAGGGGAAGGAGTTCGAGTGCAAGGTCAACAACGAAGA | |
| | CCTCCCAGGCCCCATCACGAGGACCATCTCCAAGGCCAAAGGTGGGCAAGGT | |
| | GGGCAGGCGGAGAGGGTCCCGTGCGGCCACCTGGGGGGACCATCATGCTAA | |
| | CTGAGGTGCCTGGCTTCACAGGGGTGGTACGGAGCCCGGAGGTGTACACCCT | |
| | GCCCCCACCCGCCGAGGAGCTGTCCAAGAGCATAGTCACGCTAACCTGCCTG | |
| | GTCAAAAGCTTCTTCCCGCCTTTCATCCATGTTGAGTGGAAAATCAACGGAAAA | |
| | CCAGAGCCAGAGAACGCATACCGCACCACCCCGCCCCAGGAGGACGAGGAC | |
| | GGGACCTACTTCCTGTACAGCAAGTTCTCGGTGGAAAAGTTCAGGTGGCACAG | |
| | TGGAGGCATCCACTGTGCGGTGATGCACGAGGCTCTGCACAACCACTACACC | |
| | C | |
| IgG6a | GCCCCCAAGACGGCCCCATCGGTCTACCCTCTGGCCCCCTGCGGCAGGGACA | 81 |
| constant | CGTCTGGCCCTAACGTGGCCTTGGGCTGCCTGGCCTCAAGCTACTTCCCCGA | |
| heavy chain | GCCAGTGACCCTGACCTGGAACTCGGGCGCCCTGACCAGTGGCGTGCATACC | |
| | TTCCCATCCGTCCTGCAGCCGTCAGGGCTCTACTCCCTCAGCAGCATGGTGAC | |
| | CGTGCCGGCCAGCAGCCTGTCCAGCAAGAGCTACACCTGCAATGTCAACCAC | |
| | CCGGCCACCACCACCAAGGTGGACCTGTGTGTTGGTGAGTGCCTGGGCTGGG | |
| | GGCAGGTGTGTAGACGGGCTGAGGTCAGCCTTCCTGCCGAGAGACCACCCCA | |
| | CCAGGGTGTCAGGGCCGCTCACCTCTGTCCCCCTCACCTGCAGGCCCAGGCT | |
| | CAGGGAGGGGTCAACTCGGCACTTCGGAGAGGCCAGGGGGGCACAGGCGG | |
| | GACCCCTCCCCCGGCCCTGGGCCCCCAGCAGCACGAGCTGCTCTGGGATGTA | |
| | CCAAGACCTGACCCTGACCTGAGCCCAACCCTGGACCCTCCCTACCCCCAGT | |
| | GACTCTGTGTGTTCTCTCCTCAGGACGACCATGTCCCATATGCCCAGGTAAGA | |
| | AACTCGGGCTTCGCCCTCCTCCCCAAGGAGGTAGCCTGAGCCCAGGTTCGGC | |
| | CCGAGGGGGGACAGGTGCCCAGGAGGACCCCAGGCCACGAGCTGACCCGCA | |
| | CTCTGTCTCCCCCACCAGCCTGTGAAGGGCCCGGGCCCTCGGTCTTCATCTTC | |
| | CCTCCAAAACCCAAGGACACCCTCATGATCTCCCGGACACCCCAGGTCACGTG | |
| | CGTGGTGGTAGATGTGAGCCAGGAAAACCCGGAGGTCCAGTTCTCCTGGTAT | |
| | GTGGACGGTGTAGAGGTGCACACGGCCCAGACGAGGCCAAAGGAGGCGCAG | |
| | TTCAACAGCACCTACCGTGTGGTCAGCGTCCTGCCCATCCAGCACGAGGACTG | |
| | GCTGAAGGGGAAGGAGTTCGAGTGCAAGGTCAACAACAAAGACCTCCCAGCC | |
| | CCCATCACAAGGATCATCTCCAAGGCCAAAGGTGGGCAAGGGGGGCAAGGGG | |
| | GGCAGGCGGAGAGGGACCTGTGGGGCCACCTGGGGGGACCATCATGCTAAC | |
| | AGAGGTGCCTGGCTTCACAGGGCCGAGCCGGGAGCCGCAGGTGTACACCCT | |
| | GTCCCCATCCGCCGAGGAGCTGTCCAGGAGCAAAGTCAGCATAACCTGCCTG | |
| | GTCACTGGCTTCTACCCACCTGACATCGATGTCGAGTGGAAGAGCAACGGACA | |
| | GCCGGAGCCAGAGGGCAATTACCGCACCACCCCGCCCCAGCAGGACGTGGA | |
| | CGGGACCTACTTCCTGTACAGCAAGCTCGCGGTGGACAAGGCCAGCTGGCAG | |
| | CGTGGAGACCCATTCCAGTGTGCGGTGATGCACGAGGCTCTGCACAACCACTA | |
| | CACCCAGAAGTCCATCTTCAAGACTCCGGGTAAATGAGCCACTTGCACCCCAC | |
| | GTGCTCTCGGGTCCCACGAGCTCGCCTGAGCCCCAGCCCTGTGTACATACGT | |
| | CCCGGGCCAGCATGAAATAAA | |
| IgG6b | GCCCCCAAGACGGCCCCATCGGTCTACCCTCTGGCCCCCTGCGGCAGGGACA | 82 |
| constant | CGTCTGGCCCTAACGTGGCCTTGGGCTGCCTGGCCTCAAGCTACTTCCCCGA | |
| heavy chain | GCCAGTGACCGTGACCTGGAACTCGGGCGCCCTGACCAGTGGCGTGCACACC | |
| | TTCCCATCCGTCCTGCAGCCGTCAGGGCTCTACTCCCTCAGCAGCACGGTGAC | |
| | CGTGCCGGCCAGGAGCTCGTCCAGAAAGTGCTTCACGTGCAATGTCAACCAC | |
| | CCGGCCACCACCACCAAGGTGGACCTGTGTGTTGGTGAGTGCCTGGGCTGGG | |
| | GGCAGGTGTGTAGACGGGCTGAGGTCAGCCTTCCTGCCGAGAGACCACCCCA | |
| | CCAGGGTGTCAGGGCCGCTCACCTCTGTCCCCCTCACCTGCAGGCCCAGGCT | |
| | CAGGGAGGGGTCAACTCGGCACTTCGGAGAGGCCAGGGTGGGCACAGGCGG | |
| | GACCCCTCCCCCGGCCCTGGGCCCCCAGCAGCACGAGCTGCTCTGGGATGTA | |
| | CCAAGACCTGACCCTGACCTGAGCCCAACCCTGGACCCTCCCTACCCCCAGT | |
| | GACTCTGTGTGTTCTCTCCTCAGGACGACCATGTCCCATATGCCCAGGTAAGA | |
| | AACTCGGGCTTCGCCCTCCTCCCCAAGGAGGTAGCCTGAGCCCAGGTTCGGC | |
| | CCGAGGGGGGACAGGTGCCCAGGAGGACCCCAGGCCACGAGCTGACCCGCA | |
| | CTCTGTCTCCCCCACCAGCCTGTGAAGGGAACGGGCCCTCGGTCTTCATCTTC | |
| | CCTCCAAAACCCAAGGACACCCTCATGATCTCCCGGACCCCCGAGGTCACGTG | |
| | CGTGGTGGTAGATGTGAGCCAGGAAAACCCGGAGGTCCAGTTCTCCTGGTAC | |
| | GTGGACGGCGAAGAGGTGCACACGGCCGAGACGAGGCCAAAGGAGGAGCAG | |
| | TTCAACAGCACCTACCGTGTGGTCAGCGTCCTGCCCATCCAGCACCAGGACTG | |
| | GCTGAAGGGAAAGGAGTTCGAGTGCAAGGTCAACAACAAAGACCTCCCAGCC | |
| | CCCATCACAAGGATCATCTCCAAGGCCAAAGGTGGGCAAGGGGGGCAAGGGG | |
| | GGCAGGCGGAGAGGGACCTGTGGGGCCACCTGGGGGGACCATCATGCTAAC | |
| | AGAGGTGCCTGGCTTCACAGGGCCGAGCCGGGAGCCGCAGGTGTACACCCT | |
| | GTCCCCATCCGCCGAGGAGCTGTCCAGGAGCAAAGTCAGCATAACCTGCCTG | |
| | GTCACTGGCTTCTACCCACCTGACATCGATGTCGAGTGGAAGAGCAACGGACA | |
| | GCCGGAGCCAGAGGGCAATTACCGCTCCACCCCGCCCCAGGAGGACGAGGA | |
| | CGGGACCTACTTCCTGTACAGCAAACTCGCGGTGGACAAGGCGAGGTTGCAG | |
| | AGTGGAGGCATCCACTGTGCGGTGATGCACGAGGCTCTGCACAACCACTACA | |
| | CCCAGAAGTCCATCTCCAAGACT | |
| IgA constant | CGGGCTGATGCCAAGCCATCCGTCTTCATCTTCCCGCCATCGAAGGAGCAGTT | 71 |
| light chain | AGAGACCCAAACTGTCTCTGTGGTGTGCTTGCTCAATAGCTTCTTCCCCAGAGA | |
| | AGTCAATGTCAAGTGGAAAGTGGATGGGGTGGTCCAAAGCAGTGGCATCTTGG | |
| | ATAGTGTCACAGAGCAGGACAGCAAGGACAGCACCTACAGCCTCAGCAGCAC | |
| | CCTCTCGCTGCCCACGTCACAGTACCTAAGTCATAATTTATATTCCTGTGAGGT | |
| | CACCCACAAGACCCTGGCCTCCCCTCTGGTCAAAAGCTTCAACAGGAACGAGT | |
| | GTGAGGCT | |
| IgA constant | AGCGAGACCTCTCCAAAGATCTTCCCACTGACACTGGGATCTTCCGAGCCAGC | 83 |
| heavy chain | TGGCTACGTGGTCATCGCCTGTCTGGTGAGGGACTTCTTTCCTTCCGAGCCAC | |
| | TGACCGTGACATGGTCCCCTAGCAGGGAGGGCGTGATCGTGCGGAATTTTCC | |
| | ACCAGCTCAGGCTGGAGGACTGTATACCATGAGCTCTCAGCTGACACTGCCTG | |
| | TGGAGCAGTGCCCAGCCGATCAGATCCTGAAGTGTCAGGTGCAGCATCTGAG | |
| | CAAGTCCAGCCAGTCTGTGAATGTGCCCTGCAAGGTGCTGCCTTCTGACCCCT | |
| | GTCCTCAGTGCTGTAAGCCTTCTCTGTCCCTGCAGCCTCCAGCTCTGGCCGAT | |
| | CTGCTGCTGGGCAGCAACGCTTCTCTGACCTGCACACTGAGCGGCCTGAAGA | |
| | AGTCTGAGGGCGTGTCCTTCACCTGGCAGCCATCTGGAGGCAAGGACGCTGT | |
| | GCAGGCTTCCCCAACAAGGGATAGCTGCGGATGTTACTCCGTGTCCTCCATCC | |
| | TGCCAGGATGCGCTGACCCATGGAACAAGGGAGAGACATTCTCCTGTACCGCT | |
| | GCCCACAGCGAGCTGAAGTCTGCTCTGACCGCCACAATCACCAAGCCCAAGG | |
| | TGAACACCTTTAGACCTCAGGTGCATCTGCTGCCCCCTCCAAGCGAGGAGCTG | |
| | GCTCTGAATGAGCTGGTGACACTGACCTGTCTGGTGAGAGGCTTTTCTCCTAA | |
| | GGACGTGCTGGTGCGCTGGCTGCAGGGAGGACAGGAGCTGCCAAGGGATAA | |
| | GTACCTGGTGTGGGAGTCCCTGCCAGAGCCAGGACAGGCTATCCCAACATAT | |
| | GCTGTGACCAGCGTGCTGAGGGTGGACGCTGAGGATTGGAAGCAGGGCGACA | |
| | CCTTCAGCTGCATGGTGGGACACGAGGCTCTGCCACTGGCCTTTACACAGAAG | |
| | ACCATCGATAGGCTGGCTGGCAAGCCTACACATGTGAACGTGTCTGTGGTCAT | |
| | GGCTGAGGCCGAGGGCATCTGTTAC | |

### Examples

The above aspects and embodiments are further supported by the following non-limiting examples.

### Material and Methods

### Generation of Monoclonal Antibodies

Immunizations with porcine kidney APN (Sigma) and hybridoma generation were carried out by Monash University. Mother clones were subcloned and 6 different clones were selected and further expanded. Secreted antibodies were subsequently purified from the culture supernatant by protein G affinity chromatography (GE healthcare). Monoclonal antibody isotypes were determined using the mouse IgG isotyping ELISA kit (Iso-2, Sigma).

A vector coding for the α-APN-mIgG1-FedF and the α-APN-mIgG2a-FedF fusion antibody was generated by Genscript. Briefly, the heavy chain of an APN-specific mouse monoclonal antibody (clone IMM013 or clone C5C8 as a comparative example) was fused to the tip adhesin FedF₁₅₋₁₆₅ of F18 fimbriae (PDB entry: 4B4P) using a (G4S)3-flexible linker and cloned into MCS2 of the pVITRO1-neo-mcs vector using CloneEZ® seamless cloning technology. Then, the light chain of the same clone (IMM013 or C5C8) was cloned into MCS1 of the same vector to get the final α-APN-mIgG1-FedF or α-APN-mIgG2a-FedF expression vector. After stable transfection into CHO cells, the best producing clones were selected by serial dilution and further expanded. Secreted antibodies were purified from cell culture supernatant using protein A affinity chromatography (GE Healthcare). The chimeric α-APN-pIgA-FedF and pIgA-FedF control construct were generated as described previously, using the variable regions of the IMM013 clone and the porcine constant light (Acc. No. AAA03520.1) and porcine IgA heavy (Acc. No. AAA65943.1) chains (incorporated by reference Van der Weken H. 2019). The pIgA-FedF control construct was derived from the IMM013 clone, but contained a single mutation (G100D; MUT7) in the CDR3H region, resulting in loss of binding towards APN (Fig. S1b). Secreted antibody was purified using ammonium sulphate precipitation between 40 and 46% saturation and dialyzed against PBS.

### Affinity Measurements

Affinity measurements were performed using bio-layer interferometry (BLI; Octet RED96). Here, 10 μg/ml of the ligand (biotinylated porcine APN; 1:3 ratio) was first bound on a high precision streptavidin (SAX) biosensor soaked in PBS, followed by the addition of the analyte (mAbs) at 100 nM in PBS+0.2% Tween-20+1% BSA (PBST+BSA). Analyzed data was fitted with a 1:1 local full fit. For epitope binning, ligand (biotinylated APN) was first bound on a SAX biosensor soaked in PBS, followed by binding of primary Abs at 500 nM in PBST+BSA. Next, secondary Abs were added at 250 nM in PBST+BSA. Data were analyzed with high-throughput epitope binning software.

### APN-Specific Binding Assays

Binding of mAbs towards purified APN was performed with ELISA as described (Bakshi S. 2020). Binding of mAbs towards membrane-bound APN on BHK-APN cells was analyzed by flow cytometry (Cytoflex, Beckman Coulter) as described (Bakshi S. 2020), with slight modifications. Briefly, cells were incubated with mAbs (10 μg/ml) and detected with a fluorescein isothiocyanate (FITC)-conjugated sheep α-mouse IgG (whole molecule) F(ab′)2 fragment (1:100 dilution) (Merck, F2883). Isotype control mouse IgG1 and IgG2a antibodies (in-house) were used as controls.

### Porcine Small Intestinal Explants

Tissue explants from porcine ileum were obtained as described (Baert K. 2015). Antibodies (40 μg) were added to the explants for 30 minutes at 37° C. and 5% CO₂. Upon this incubation period, the explants were washed with PBS, placed in methocel, snap frozen in liquid nitrogen and stored at −80° C. until use.

### Gut Ligated Loop Experiments

In total, six female, 5-week-old piglets were used to assess the uptake of α-APN-mIgG1 (clone IMM013) in gut ligated loops as described (Loos M. 2013). Three of these animals were used in a preliminary study to locate the mesenteric lymph nodes draining each area of the gut and to study the kinetics of the uptake in the gut ligated loops after different incubation times. Briefly, following anesthesia and laparotomy, the jejunum was localized and three 3 cm loops with 20 cm intervals between each loop were made avoiding Peyer's patches. Blood supply was assured by placing the ligatures between the mesenteric arcades. For the location of the draining MLN, 5% Evans Blue was injected subserosally between the ligatures of each loop of the small intestine. One milligram of fluorescently labelled (DyLight™ 755, Thermo Fisher Scientific) α-APN mAb (clone IMM013) or an IgG1 isotype control (in house; clone 19C9) (Cliquet P 2001) were diluted in 3 ml PBS and injected in the lumen of the loops. A loop injected with 3 ml PBS was used as a negative control. Upon injection, each loop was returned to the abdominal cavity and the abdomen was closed. After a 5 h incubation, the animals were euthanized with an overdose of sodium pentobarbital 20% (60 mg/2.5 kg; Kela) and tissue samples were collected. Loops and draining MLN were imaged using an IVIS Lumina II fluorescent imaging system. Tissues were kept on ice protected from light until imaging. Following, tissue samples were embedded in 2% Methocel® MC (Fluka), snap frozen in liquid nitrogen and stored at −80° C. until use.

### Immunohistochemistry

For the endocytosis experiments using the BHK-APN cell line, cells (1.0×105 cells/well in 1 ml culture medium) were seeded in 24-well plates on top of a sterile cover slip and incubated until a monolayer was formed. Cells were washed twice with ice-cold PBS and stored on ice before the α-APN-mIgG1 (40 μg/ml) was added. After 60 min incubation at 4° C., cells were washed 3 times with ice-cold PBS+1% FCS and incubated for 30 min at 37° C., 5% CO2 in warm culture medium. Before or after incubation at 37° C., cells were washed twice with ice-cold PBS and fixated for 10 min with 500 μl 4% paraformaldehyde. Next, presence of the antibody on the cell membrane was detected with an AF568-conjugated α-mouse IgG(H+L) (2 μg/ml; Invitrogen, A-11004) for 30 min at room temperature (RT). After three washes with PBS+1% FCS, cells were permeabilized with 250 μl 0.2% Triton-X100 for 2 min and washed 3 times with PBS+1% FCS. Intracellular α-APN-mIgG1 was then detected using a FITC-conjugated sheep α-mouse IgG F(ab′)2 fragment (1:100 dilution; Merck, F2883) for 1 h at RT. The nucleus was counterstained with Hoechst (10 μg/ml) for 2 min. After three washes, the cover slip was mounted on a microscope slide in mounting solution (Dabco).

For staining of tissue sections, cryosections (10 μm) were cut with a cryotome (Leica CM3050 S), placed on APES-coated glass slides and fixated in aceton for 10 minutes at −20° C. Tissue sections were then washed with 50 mM ammonium chloride (pH 8.0) for 30 min followed by a short PBS wash. Next, tissue sections were blocked with PBS+10% sheep serum or goat serum for 30 minutes in a humid cell at 37° C. To assess binding of the different mAbs, sections were incubated for 1 h at 37° C. with these mAbs (10 μg/ml). After incubation, a secondary FITC-conjugated sheep α-mouse IgG F(ab′)2 fragment (1:100 dilution; Merck, F2883) was added for 1 h at 37° C. For staining and the α-APN-mIgG1 uptake experiment with explant tissue, a rabbit pAb to wide-spectrum cytokeratin (1:100 dilution; Abcam, ab9377) was added for 1 h at 37° C., followed by a secondary FITC-conjugated sheep α-mouse IgG F(ab′)2 fragment (1:100 dilution; Merck, F2883) and a Texas Red-conjugated goat α-rabbit IgG(H+L) (1:100 dilution, Invitrogen). To stain immune cells, mAbs to MHC-II (clone MSA3, IgG2a, 15 μg/ml, in house), CD11R1 (biotinylated, clone MIL4, IgG1, 15 μg/ml, Bio-Rad) and CD172a (biotinylated, clone 74-22-15a, IgG1, 10 μg/ml, in house) were added and incubated for 1 h at 37° C., followed by another incubation for 1 h at 37° C. with FITC-conjugated sheep α-mouse IgG2a (Invitrogen, Catalog #31634, 1/100 dilution) or streptavidin-Texas Red (Invitrogen, S872, 1/50 dilution).

Slides were washed with PBS between each step, counterstained with Hoechst (10 μg/ml) for 2 min and mounted on a microscope slide in mounting solution (Dabco). Images of explants were taken with a confocal microscope (Leica). Other images were taken with a fluorescent microscope (Leica). Images were analyzed and processed using Fiji.

### Animals and Immunization Procedures

### C5C8

C5C8 is used as a comparative example for IMMO13. Nine conventionally reared piglets (Belgian Landrace×Pietrain) from a Belgian farm were weaned at 3 weeks and transported to our facilities. These animals were screened to be F18 fimbriae and cholera toxin seronegative. All piglets were F18-receptor positive as determined by FUT1 PCR. The piglets were housed in isolation units and treated with colistin (Colivet quick Pump®, 6.4 mg/kg bodyweight) for 5 days before the start of the experiment.

Animals were randomly divided in 3 groups of three animals:
1) a mouse IgG2a (mIgG2a) isotype control antibody
2) an APN-targeted mouse IgG2a mAb (α-APN-mIgG2a)
3) an α-APN-mIgG2a-FedF fusion construct

The piglets orally immunized on three consecutive days followed by a booster immunization 14 days post primary immunization (dppi) with either a control antibody (IgG2a), an APN-targeted antibody (C5C8) or an APN-targeted antibody-FedF fusion construct (C5C8-FedF). All oral immunizations were adjuvanted with 50 μg cholera toxin (Merck, C8052). The gastric pH was neutralized by administration of Omeprazole (20 mg) 24 hours before each immunization and animals were deprived of feed and water 3 hours before the immunizations. Animals were immunized by oral administration with a conventional syringe containing 1 mg antibody in 10 ml PBS. Blood was collected at 0, 9, 14, 21 and 28 dppi to analyse serum antibody responses by ELISA. At 28 dppi animals were euthanized by intravenous injection of sodium pentobarbital 20% (60 mg/2.5 kg; Kela).

### IMM013

Twenty-five conventionally reared piglets (Belgian Landrace×Pietrain) from a Belgian farm were weaned at 3 weeks and transported to our facilities. These animals were screened to be mouse IgG1, cholera toxin and F18 fimbriae seronegative. Piglets receiving the FedF constructs were also screened to be F18 receptor positive using FUT1 genotyping (Meijerink E. 1997). The piglets were housed in isolation units and treated with colistin (Colivet quick Pump®, 6.4 mg/kg bodyweight) for 5 days before the start of the experiment. Animals were randomly divided in five groups of 5 animals:
1) a mouse IgG1 (migG1) isotype control mAb (clone 19C9),
2) an APN-targeted mouse IgG1 mAb (α-APN-mIgG1),
3) an α-APN-mIgG1-FedF fusion construct and
4) the α-APN-pIgA-FedF or
5) pIgA-FedF chimeric mouse-porcine IgA fusion constructs.

The piglets were orally immunized on three consecutive days followed by a booster immunization 14 days post primary immunization (dppi). All immunizations were adjuvanted with 50 μg cholera toxin (Merck, C8052). The gastric pH was neutralized by administration of Omeprazole (20 mg) 24 hours before each immunization and animals were deprived of feed and water 3 hours before the immunizations. Animals were immunized by oral administration with a syringe with 1 mg migG1 isotype control or α-APN-mIgG1 and 1.2 mg α-APN-mIgG1-FedF, α-APN-pIgA-FedF or pIgA-FedF in 10 ml PBS to account for equimolar ratios. Blood was collected at 0, 9, 14, 21 and 28 dppi to analyze serum antibody responses by ELISA and assess the presence of antigen-specific IgA+ antibody secreting cells (ASC) in the peripheral blood mononuclear cell (PBMC) population. At 28 dppi animals were euthanized by intravenous injection of sodium pentobarbital 20% (60 mg/2.5 kg; Kela) and upon exsanguination intestinal tissues were collected.

Antigen-Specific Serum Antibody Responses Blood was taken from the jugular vein into a gel and clot activator tube (Vacutest, Kima). After 1 h incubation at RT, tubes were centrifuged and serum was collected, inactivated at 56° C. for 30 minutes and kaolin treated. Serum samples were stored at −20° C. until use. Maxisorp microtiter plates (96-well, Life Technologies) were coated with mouse IgG1 monoclonal antibody (19C9 or IMM013, 6 μg/ml) or FedF (in house, 5 μg/ml) in PBS for 2 h at 37° C. FedF was purified as described previously (Baert K et al. 2015). Upon overnight blocking at 4° C. in PBS supplemented with 0.2% Tween80 and 3% BSA, the serially diluted serum samples were added in dilution buffer (PBS+0.2% Tween20+3% BSA) to the wells. Upon incubation for 1 h at 37° C., plates were washed and incubated for 1 h at 37° C. with HRP-conjugated mouse α-pig IgG (1/1000; MabTech; Nacka Strand, Sweden) or IgA (1/10000; Bethyl; Montgomery, Texas, U.S.). Following 3 washes, ABTS was added and the optical density was measured at 405 nm after 60 min incubation at 37° C. using a spectrophotometer (Tecan SpectraFluor). Serum was serially diluted starting at 1/30 for IgG1 and IMM013 responses and 1/10 for FedF serum responses. Titer values were obtained by calculating the non-linear regression curve and using a cut off value 0.2.

### Antigen-Specific Antibody Secreting Cells in the Intestinal Tissues

Mononuclear cells (MCs) were isolated from blood (PBMC), mesenteric lymph nodes (MLN), jejunal Peyer's Patches (JPP), jejunal lamina propria (JLP), ileal Peyer's Patches (IPP) and ileal lamina propria (ILP) and processed as described (Verdonck F. 2002; Devriendt B. 2009). The obtained cell suspensions were filtered through a 70 μm cell strainer and the MCs were isolated by density gradient centrifugation on Lymphoprep (Alere Technologies, Oslo, Norway) for 25 minutes at 800 g and 18° C. Isolated MCs were resuspended at 2.5×106 cells/ml (PBMC and MLN) or 5×106 cells/ml (other tissues) in CTL-Test™ B-medium (Cellular Technology Limited, Cleveland, USA). MultiScreen filter plates (96-well format, MAIPA4510, Millipore) were activated with 70% ethanol for 30 seconds, washed twice with ultrapure (UP) water and coated overnight at 4° C. with 10 μg/ml mouse IgG1 (in house) or 10 μg/ml FedF. Upon washing, the plates were incubated for 2 h at 37° C. with CTL-test B medium. Mononuclear cells (5×105 cells/well) from each tissue were added to the wells and incubated for 18 h at 37° C., 5% CO2 in a humidified atmosphere. Cells were then removed by intensive washing with PBS containing 0.1% Tween20. Upon washing, HRP-conjugated α-pig IgG (1/1000; MabTech) or IgA (1/10000; Bethyl) was added in assay buffer (PBS containing 0.1% Tween20 and 0.1% BSA) and incubated for 1 hour at RT. Finally, 3,3′,5,5′-Tetramethylbenzidine (TMB) substrate for membranes (Sigma) was added to the wells after three washes. The reaction was stopped by intensive washing with UP water and the plates were allowed to dry overnight at 4° C. Images were taken using an immunospot reader (Luminoskan) and spots were counted manually.

### Data Analysis

The data were analyzed using GraphPad Prism software version 7. Differences in the frequency of ASCs between different groups were analyzed using the Kruskal-Wallis test. Serum responses between groups and between days were analyzed using a Two-way ANOVA with repeated measures. Multiple comparisons were corrected using the Two-stage linear step-up procedure of Benjamini, Krieger and Yekutieli. Differences were considered significant when the adjusted p-value<0.05.

### In Vivo Challenge Experiment

Sixteen conventionally reared piglets (Belgian Landrace×Pietrain) from a Belgian farm were weaned at 3 weeks and transported to our facilities. These animals were screened to be F18 fimbriae seronegative and F18 receptor positive using FUT1 genotyping (Meijerink E., 1997). The piglets were housed in isolation units and treated with colistin (Colivet quick Pump®, 6.4 mg/kg bodyweight) for 5 days before the start of the experiment.

Animals were randomly divided in two groups of 8 piglets each.
A) a PBS control group
B) the aAPN-pIgA-FedF vaccine group

The piglets were orally immunized on three consecutive days followed by a booster immunization 14 days post primary immunization (dppi). Oral immunization was performed using a syringe containing 3 mg of the α-APN-pIgA-FedF in 10 ml PBS and adjuvanted with 50 μg cholera toxin (Merck, C8052). The PBS control group received only PBS and no cholera toxin adjuvant. The gastric pH was neutralized by administration of omeprazole (20 mg) 24 hours before each immunization and animals were deprived of feed and water 3 hours before and 2 hours after the immunizations. At 28 dppi, the animals were challenged with an F18-fimbriated STEC strain (F107/86) as described (Coddens et al., 2017). In brief, the piglets were first sedated with Stressnil (2 mg/kg bodyweight) and gastric pH was neutralized by intragastric administration of 60 ml NaHCO₃ (1.4% in distilled water), followed by intragastric administration of 1011 F107/86 in 10 ml sterile PBS. Fecal samples were collected daily from 1 to 9 days post challenge (dpc) to determine the excretion of the F107/86 strain. Hereto, 100 μl of 4 serial 10-fold dilutions in PBS, starting from a 1% (w/v) suspension, was plated onto blood agar plates, supplemented with 1 mg/ml streptomycin sulfate salt (Sigma). After growing overnight, F18+ E. coli were identified using dot blotting and immune detection with IMM02 (anti-FedA mAb; in house) and an HRP-conjugated anti-mouse IgG (Dako) (Tiels P., 2007). Binding of the secondary antibody was visualized with a 3-amino-9-ethylcarbazole solution. Results are represented as the mean log 10 number±the standard deviation of excreted colony forming units (CFU) per gram of feces.

Statistical analysis was performed using GraphPad Prism 9 software (GraphPad Software Inc.). A mixed-effects model with Geisser-Greenhouse correction was performed to analyze the fecal F18+ E. coli excretion between groups, using the treatment and time as variables. To correct for multiple comparisons, the false discovery rate was controlled using the two-stage linear step-up procedure of Benjamini, Krieger and Yekutieli. p<0.05 was considered as statistically significant.

### Results

### Characterization of APN-Specific Monoclonal Antibodies

Using standard techniques, 6 hybridoma clones were obtained and further characterized. Although these clones recognized APN in an initial screening, upon their purification clone H2F2 failed to recognize porcine APN in ELISA. Clone H2B8 showed the strongest binding, while IMM013 showed the weakest binding, with optical density (O.D.) values barely above the detection limit (FIG. 1a). Next, flow cytometry analysis was performed using an APN-expressing cell line (BHK-APN). Surprisingly, all monoclonal antibodies showed a similar binding profile as compared to ELISA, except for IMM013 (FIG. 1b). While the latter was barely detectable in ELISA, it showed the best binding to membrane bound APN, indicating that purified kidney APN might differ from membrane-bound APN in epitope accessibility. As clone H2F2 also did not bind to BHK-APN cells, it was excluded from further analyses. The affinity of the remaining clones was determined using bio-layer interferometry (BLI) (FIG. 1c). These results were similar to flow cytometry with IMM013 having the strongest affinity (KD) value in the low nanomolar range i.e. 1.6 nM (FIG. 1c). In an effort to determine if these mAbs bind different epitopes, an epitope binning experiment was performed using IMM013, C5C8, F1B7 and polyclonal rabbit α-APN antibodies as a positive control (FIG. 1d). As expected, polyclonal α-APN antibodies were able to block binding of all mAbs to APN, while IMM013 blocked binding of C5C8 and F1B7 to APN. Surprisingly, these latter two were not able to block IMM013 binding.

As these monoclonal antibodies might be used for the delivery of vaccine antigens to the small intestinal epithelium, we assessed their ability to recognize APN on small intestinal jejunum and ileum. As shown in Table 4, IMM013 showed the best binding to APN present on the apical side of the small intestinal enterocytes. H2B8, F1B7 and H1H6 showed an intermediate binding, while C5C8 showed a very weak binding (Table 4). These binding profiles were very similar to our flow cytometry data.

TABLE 4
Binding profile of APN-specific monoclonal
antibodies to small intestinal APN of pig.

| Small | Isotype | | | | | | |
|---|---|---|---|---|---|---|---|
| intestine | ctrl | Polyclonal | IMMO13 | H2B8 | F1B7 | H1H6 | C5C8 |
| Jejunum | − | ++ | ++ | + | + | + | +/− |
| Ileum | − | ++++ | +++ | + | + | + | +/− |

### In Vitro and In Vivo Behavior of APN-Targeted mAb

Because only IMM013 showed a strong binding profile to small intestinal APN, this monoclonal antibody was further evaluated for its ability to serve as an antigen delivery system. Using cell lines and gut explants, the uptake of IMM013 was assessed. In contrast to an irrelevant mouse IgG1, IMM013 was clearly taken up by BHK-APN cells and by small intestinal enterocytes in the explants. Some transcytosis of IMM013 occurred as can be seen by the presence of antibodies at the basolateral side of the intestinal epithelial cells. To confirm the behavior of IMM013 in an in vivo setting, gut ligated loop experiments were performed. Since we wanted to assess if APN targeted antibodies can reach the mesenteric lymph nodes (MLN) upon epithelial transcytosis, Evans blue was injected at the edges of the gut loops to identify the draining MLN of each ligated loop. Upon injection of DL755-labelled IMM013, its presence in the gut loop and the draining MLN was confirmed upon 5h incubation. Similar to the explant results, APN targeting resulted in the endocytosis and transcytosis of the antibodies by small intestinal epithelial cells. Moreover, transcytosis of IMM013 by epithelial cells resulted in the presence of mAb positive cells in the subepithelial tissue in the villi, implying that antigen presenting cells (APCs) phagocytosed the antibody released by the epithelial cells upon transcytosis. Furthermore, we also analyzed the distribution of these antibody-positive APCs in the draining MLN, where they were found mainly in the subcapsular and interfollicular regions. To further investigate which cells might phagocytose the antibody upon epithelial transcytosis, tissue sections were stained with three APC markers associated with mononuclear phagocytes in the porcine gut: MHC-II, SIRP-α and CD11R1. The results showed that 98% of the IMM013 positive cells expressed MHC class II, 96% expressed SIRP-α and 93% expressed CD11R1.

Antigen-Specific Intestinal Immune Responses after Oral Administration of APN-Targeted Antibody Constructs

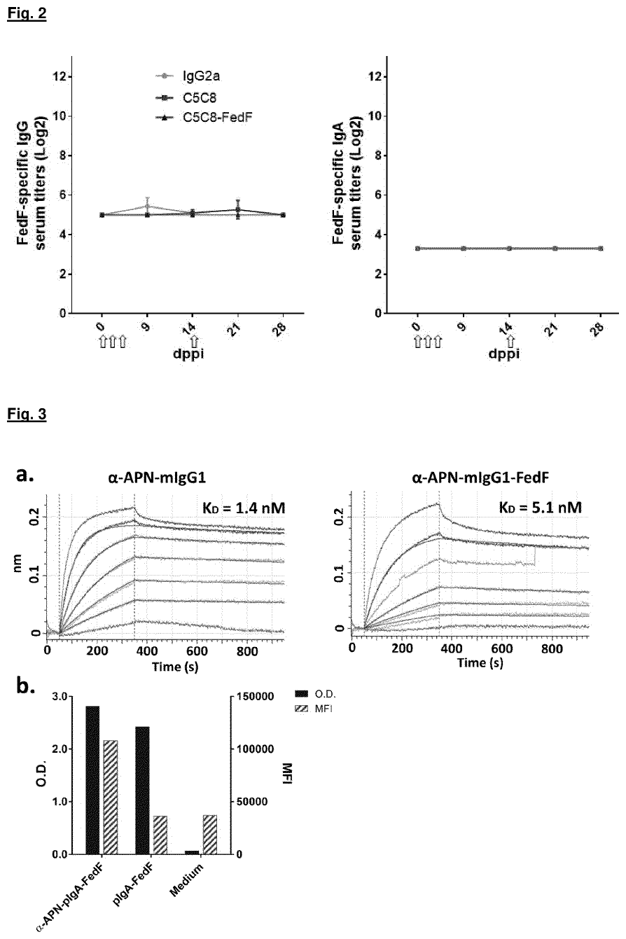

To evaluate the ability of C5C8 to induce systemic immune responses against a linked antigen after oral delivery, a fusion construct was made using the clinically relevant antigen FedF from F18 fimbriated E. coli (α-APN-mIgG2a-FedF). Piglets were then orally immunized with either a mouse IgG2a control antibody, an APN-specific mouse IgG2a antibody (α-APN-mIgG2a) or an α-APN-mIgG2a-FedF fusion construct α-APN-mIgG2a-FedF). No significant increases in FedF-specific IgG or IgA serum titers could be observed as compared to day 0 or between the different groups (FIG. 2).

To evaluate the ability of IMM013 to induce systemic and local immune responses against a linked antigen after oral delivery, several IMM013-based antibody constructs were developed. First, a fusion construct was made using the clinically relevant antigen, FedF from F18 fimbriated E. coli (α-APN-mIgG1-FedF). Next, the mouse IgG1 (migG1) Fc-domain of this construct was changed to a porcine IgA (pIgA) Fc-domain as previously described in Van der Weken H. 2020, in an attempt to increase antibody stability in the intestinal tract and reduce mouse IgG1-specific immune responses (α-APN-pIgA-FedF). To check for the effect of APN-targeting, a pig IgA-FedF control construct (pIgA-FedF) was also derived by rational design. Here, a single amino acid (G100D; MUT7) in the CDRH3 loop was mutated, resulting in the substitution of a small non-polar amino acid into a larger polar amino acid. This single mutation completely abolished APN binding, while maintaining antibody stability. Binding and uptake characteristics of the FedF-linked fusion constructs were confirmed to be similar to IMM013 (FIG. 3).

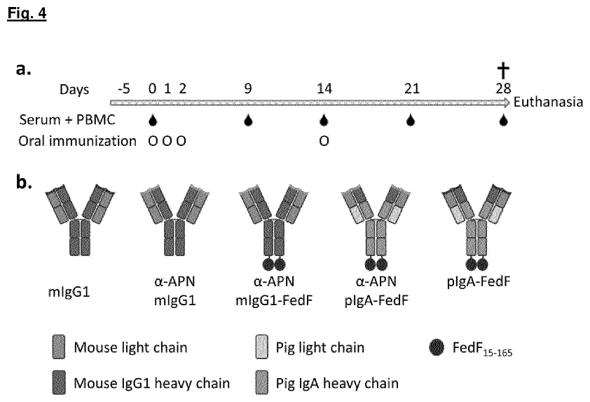

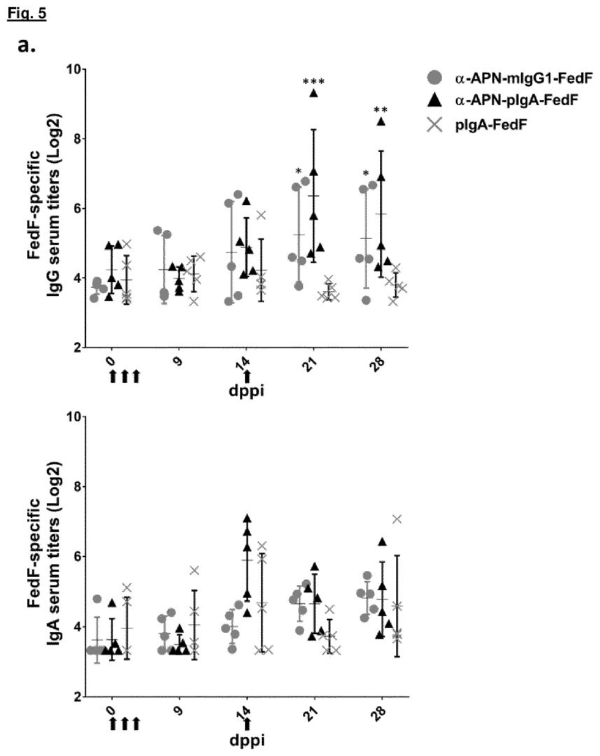

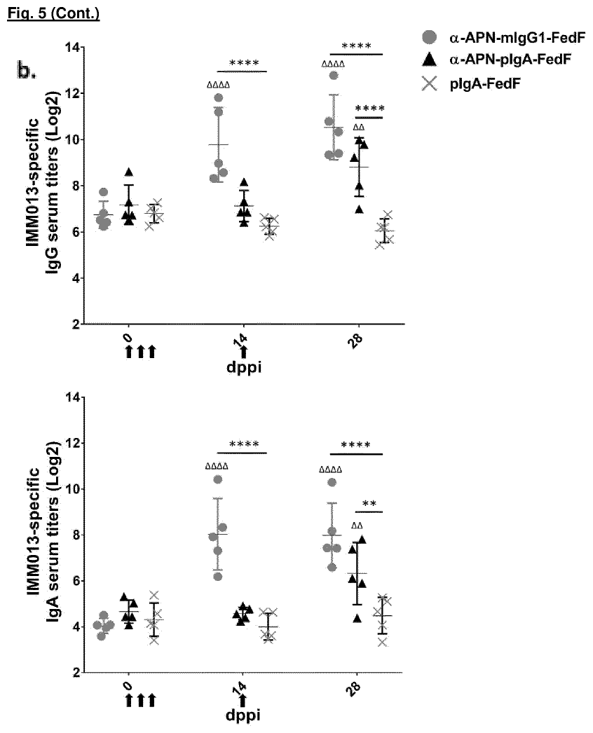

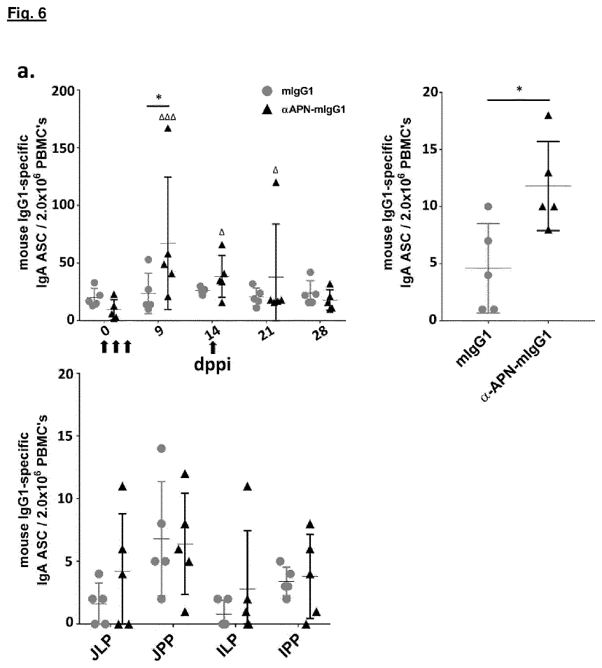

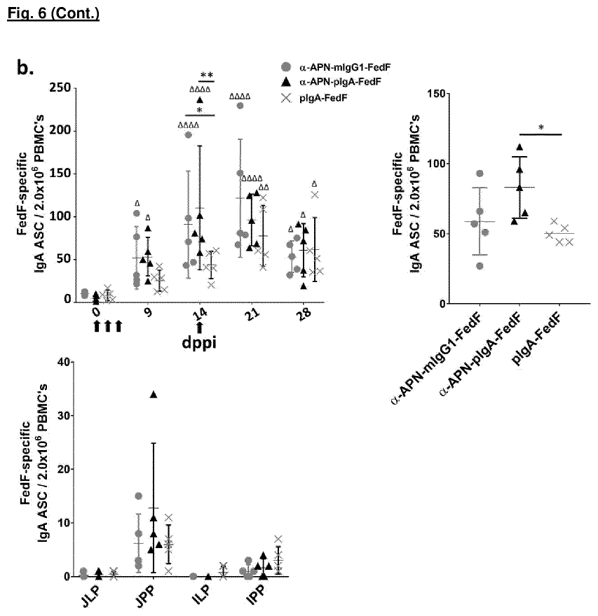

All constructs were subsequently used in an oral immunization experiment in weaned piglets to evaluate the effect of APN-targeting in inducing systemic and local immune responses against the antibody and the fused antigen. To this end, piglets were orally immunized with a mouse IgG1 isotype control, an APN-specific mouse IgG1 (α-APN-mIgG1), an α-APN-mIgG1-FedF fusion construct, a chimeric α-APN-pIgA-FedF fusion construct and a chimeric pIgA-FedF control antibody (FIG. 4). The ability of these different antibody formats to elicit mouse IgG1 and FedF-specific immune responses was evaluated by ELISA and ELispot (FIG. 5-6). Here, we showed a clear increase in mouse IgG1-specific IgG and IgA serum responses at 9, 14, 21 and 28 days post primary immunization (dppi) for the APN-targeted antibodies as compared to the migG1 isotype control. The chimeric α-APN-pIgA-FedF fusion construct did not result in mouse IgG1 serum responses.

The targeting of FedF to APN by the antibody fusion constructs also resulted in significant FedF-specific IgG serum responses at 21 and 28 dppi as compared to the pig IgA-FedF control antibody. Surprisingly, the FedF-specific IgA serum responses did not differ between groups (FIG. 5a). Although the APN-targeted IgA-FedF construct did not result in mouse IgG1-specific immune responses, significant differences in IMM013-specific serum IgG and IgA responses could be observed compared to the pIgA-FedF control antibody (FIG. 5b), indicating that the mouse variable domain is still immunogenic and that the targeting towards APN was effective in promoting immune responses.

To further investigate the mouse IgG1 and FedF-specific immune responses, the amount of circulating antigen-specific IgA+ antibody secreting cells (ASCs) were assessed by ELISpot (FIG. 6). A significant increase in the number of mouse IgG1-specific IgA ASCs was found 9 dppi for the APN targeted antibody as compared to day 0 and the mouse IgG1 isotype control (FIG. 6a). For FedF, significant increases in FedF-specific IgA ASCs as compared to day 0 were found at 9, 14, 21 and 28 dppi for the APN targeted antibody constructs, but also for the pig IgA-FedF control group at 21 and 28 dppi. Significant differences compared to the pig IgA-FedF control group could be found for the APN-targeted groups at 14 dppi (FIG. 6b). To assess local gut immune responses, the number of antigen-specific IgA+ ASCs in small intestinal tissues were enumerated by ELISpot at 28 dppi (FIG. 6). Here, APN targeting elicited both mIgG1− and FedF-specific IgA+ ASCs in the mesenteric lymph nodes.

### In Vivo Challenge Experiment

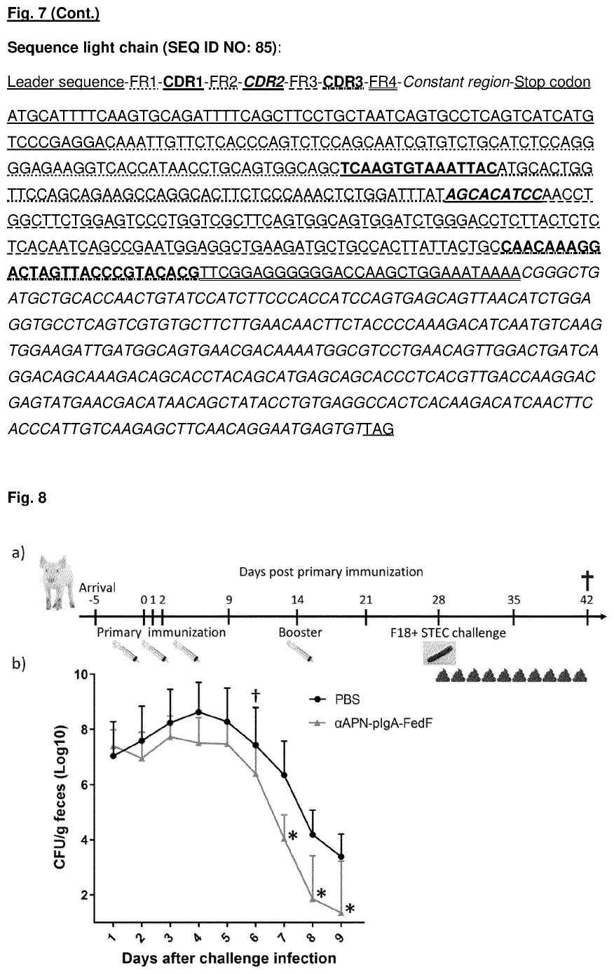

To evaluate if the observed immune responses were sufficient to protect the piglets against F18+E. coli infection, a challenge experiment was performed. First, piglets were orally immunized with the aAPN-pIgA-FedF construct or with PBS, followed by a challenge infection with an F18-fimbriated shiga-toxin producing E. coli strain (STEC) at 28 days post primary immunization (dppi). The mean fecal excretion of the F18+E. coli was subsequently monitored for 9 consecutive days (FIG. 8a). Here, we showed a significant decrease in bacterial excretion at day 7, 8 and 9 post challenge in the aAPN-pIgA-FedF immunized group as compared to the control group (FIG. 8b). One piglet in the control group had to be euthanized due to symptoms of edema disease, while no clinical signs were observed in the immunized piglets. These data demonstrate that oral immunization with an APN-targeted pIgA-FedF fusion construct provides protective immunity against a subsequent challenge infection with a FedF-expressing E. coli strain.

### CONCLUSION

In this study, we evaluated the use of APN-targeted monoclonal antibodies and recombinant antibody constructs as a delivery system for vaccine antigens.

Starting from a panel of different APN-targeting mAbs, the clone IMM013 was identified as the best candidate for further in vivo experiments. This mAb showed the strongest binding towards the membrane-bound form of APN. Affinity measurements also showed the highest values for IMM013. Interestingly, epitope binning between IMM013, F1B7 and C5C8 showed that IMM013 could block binding of the latter two mAbs, but these in turn were not able to block binding of IMM013. These data indicate that IMM013 might be able to induce structural changes upon binding, resulting in the masking of epitopes recognized by the F1B7 and C5C8 mAbs. Targeting APN using IMM013 resulted in endocytosis and transcytosis by intestinal epithelial cells as previously shown for APN-targeted polyclonal antibodies and single-domain nanobodies. Upon transcytosis, the APN-targeted IMM013 mAb was detected in subepithelial cells and in the draining mesenteric lymph nodes. Moreover, these antibody-positive subepithelial cells were also positive for MHCII, SIRP-αand CD11 R1, which are present in mononuclear phagocytes. These markers, especially CD11 R1, have been shown to be present on migratory cells from the lamina propria to the mesenteric lymph nodes in pigs. Thus, upon transcytosis by epithelial cells and phagocytosis of the released antibodies by antigen presenting cells, these cells migrate to the mesenteric lymph nodes to initiate immune responses.

In addition, we wanted to test the ability of antibody-mediated targeting of antigens towards intestinal APN to trigger antigen-specific immunity. Therefore, several APN-targeted recombinant antibody constructs were generated based on the C5C8 and IMM013 mAb and genetically linked to a clinically relevant antigen. The C5C8-based fusion construct was unable to elicit FedF-specific serum antibody responses upon oral administration to piglets. Concerning IMM013, the generated fusion constructs included an α-APN-mlgG1-FedF, a chimeric α-APN-pIgA-FedF and a chimeric pIgA-FedF not binding to APN. These constructs together with an α-APN-mIgG1 (IMM013) and a mouse IgG1 isotype control were subsequently tested in an oral vaccination experiment. As a clinically relevant antigen, the low immunogenic tip adhesin FedF of F18 fimbriated E. coli was chosen, as it previously failed to provoke any immune responses when orally administered to pigs. The fusion construct was partially porcinized with an IgA Fc-tail to minimize immune responses to the antibody itself. We opted for an IgA Fc-domain for its expected higher stability in the gut environment, even in its monomeric format. Both the α-APN-mIgG1 and α-APN-mlgG1-FedF fusion constructs generated strong mouse IgG1-specific serum IgG and IgA responses, with significant differences compared to the non-targeted mIgG1 isotype control antibody, indicating that targeting of the antibodies towards the epithelial membrane promoted immune responses.

We provide evidence that targeting of FedF towards intestinal APN also increased FedF-specific immune responses. Significant differences in FedF-specific IgG serum responses, but not IgA serum responses could be observed for the APN-targeted FedF fusion constructs as compared to the non-targeted pIgA-FedF. Interestingly, mouse IgG1- and IMM013-specific IgG serum responses were already observed 9 or 14 dppi, while significant increases in FedF-specific serum responses were only observed after the boost at 21 and 28 dppi. These data indicate that FedF itself is not a good immunogen and that a booster immunization is required to observe significant responses. Despite the lack of IgA serum responses, significant increases in the number of IgA ASCs in the PBMCs and MLNs were found as compared to the control groups.

Although no mouse IgG1-specific immune responses could be observed for the porcine IgA-FedF constructs, we could still detect significant IMM013-specific IgG and IgA serum responses for the α-APN-pIgA-FedF construct, compared to its pIgA-FedF control. These data indicate that the mouse variable domain is still immunogenic and that the targeting towards APN was effective in promoting immune responses. Although no FedF-specific IgA serum responses could be observed, we did observe significant IMM013-specific IgA serum responses.

This study showed that immunization with APN-targeted mouse IgG1-FedF and pig IgA-FedF antibodies increased FedF-specific IgG serum levels and that ASCs isolated from the draining mesenteric lymph nodes were able to secrete FedF-specific IgA. Although FedF on its own is not immunogenic when given orally, FedF-conjugates with MBP or F4-fimbriae did provide some protection against infection. In these studies, however, no increase in FedF-specific serum titers was observed. In the current study, both serum IgG titers and gut-derived IgA ASCs were increased and these correlate with protection against challenge infection.

In conclusion, we observed the antibody-mediated selective delivery of an antigen, resulted in antigen-specific systemic and local immune responses. Our results demonstrate that targeting of antigens towards the intestinal membrane receptor APN can promote both systemic and mucosal immune responses upon oral administration. We show that targeting of APN promotes uptake by the epithelial barrier and that this provides a promising platform for the delivery of biologicals towards the gut tissues and beyond.

### REFERENCES

Cox E, Deforce D, Goddeeris B, Rasschaert K, Gent University (2009). Mucosal membrane receptor and uses thereof (WO2009103555).
Melkebeek V, Rasschaert K, Bellot P, Tilleman K, Favoreel H, Deforce D, et al. Targeting aminopeptidase N, a newly identified receptor for F4ac fimbriae, enhances the intestinal mucosal immune response. Mucosal immunology. 2012; 5(6):635-45.
Baert K, de Geest B G, de Rycke R, da Fonseca Antunes A B, de Greve H, Cox E, et al. beta-glucan microparticles targeted to epithelial APN as oral antigen delivery system. Journal of controlled release: official journal of the Controlled Release Society. 2015; 220(Pt A):149-59.
Coddens A, Diswall M, Angstrom J, Breimer M E, Goddeeris B, Cox E, Teneberg S. Recognition of blood group ABH type 1 determinants by the FedF adhesin of F18-fimbriated Escherichia coli. J Biol Chem. 2009 Apr. 10; 284(15):9713-26.
Coddens A, Loos M, Vanrompay D, Remon J P, Cox E. Cranberry extract inhibits in vitro adhesion of F4 and F18+Escherichia coli to pig intestinal epithelium and reduces in vivo excretion of pigs orally challenged with F18+ verotoxigenic E. coli. Vet Microbiol. 2017 April; 202:64-71.
Haryadi R, Ho S, Kok Y J, Pu H X, Zheng L, et al. (2015) Optimization of Heavy Chain and Light Chain Signal Peptides for High Level Expression of Therapeutic Antibodies in CHO Cells. PLOS ONE 10(2): e0116878.
Van der Weken H, Cox E, Devriendt B. Rapid production of a chimeric antibody-antigen fusion protein based on 2A-peptide cleavage and green fluorescent protein expression in CHO cells. mAbs. 2019; 11(3):559-68.
Bakshi S, Sanz Garcia R, Van der Weken H, Tharad A, Pandey S, Juarez P, et al. Evaluating single-domain antibodies as carriers for targeted vaccine delivery to the small intestinal epithelium. Journal of controlled release: official journal of the Controlled Release Society. 2020; 321:416-29.
Loos M, Hellemans A, Cox E. Optimization of a small intestinal segment perfusion model for heat-stable enterotoxin A induced secretion in pigs. Veterinary immunology and immunopathology. 2013; 152(1-2):82-6.
Cliquet P, Cox E, Van Dorpe C, Schacht E, Goddeeris B M. Generation of class-selective monoclonal antibodies against the penicillin group. Journal of agricultural and food chemistry. 2001; 49(7):3349-55.
Meijerink E, Fries R, Vogeli P, Masabanda J, Wigger G, Stricker C, et al. Two alpha(1,2) fucosyltransferase genes on porcine chromosome 6q11 are closely linked to the blood group inhibitor (S) and Escherichia coli F18 receptor (ECF18R) loci. Mammalian genome: official journal of the International Mammalian Genome Society. 1997; 8(10):736-41.
Tiels P, Verdonck F, Coddens A, Ameloot P, Goddeeris B, Cox E. Monoclonal antibodies reveal a weak interaction between the F18 fimbrial adhesin FedF and the major subunit FedA. Vet Microbiol. 2007 Jan. 31; 119(2-4)
Verdonck F, Cox E, van Gog K, Van der Stede Y, Duchateau L, Deprez P, et al. Different kinetic of antibody responses following infection of newly weaned pigs with an F4 enterotoxigenic Escherichia coli strain or an F18 verotoxigenic Escherichia coli strain. Vaccine. 2002; 20(23-24):2995-3004.
Devriendt B, Gallois M, Verdonck F, Wache Y, Bimczok D, Oswald I P, et al. The food contaminant fumonisin B(1) reduces the maturation of porcine CD11R1(+) intestinal antigen presenting cells and antigen-specific immune responses, leading to a prolonged intestinal ETEC infection. Veterinary research. 2009:40(4):40.

1. An isolated antibody, or antigen-binding fragment thereof, that binds aminopeptidase N, wherein the isolated antibody comprises: (i)three heavy chain complementarity determining regions HCDR1, HCDR2, and HCDR3 as set forth in amino acid sequences SEQ ID NO: 4, SEQ ID NO: 6, and SEQ ID NO: 8, respectively and (ii) three light chain complementarity determining regions LCDR1, LCDR2, and LCDR3 as set forth in amino acid sequences SEQ ID NO: 14, SEQ ID NO: 16, and SEQ ID NO: 18, respectively.

2. The isolated antibody or antigen-binding fragment thereof according to claim 1, further comprising a heavy chain variable region (HCVR) having at least 80% amino acid sequence identity to SEQ ID NO: 2; and, optionally, a light chain variable region (LCVR) having at least 80% amino acid sequence identity to SEQ ID NO: 12.

3. The isolated antibody or antigen-binding fragment thereof according to claim 2, wherein the HCVR comprises SEQ ID NO: 2 and the LCVR comprises SEQ ID NO: 12.

4. The isolated antibody or antigen-binding fragment thereof according to claim 3, further comprising a heavy chain constant region (HCCR) having a sequence selected from SEQ ID NOs: 59-70 and a light chain constant region (LCCR) comprising SEQ ID NO: 58.

5. The isolated antibody or antigen-binding fragment thereof according to claim 1, wherein the antibody is a monoclonal antibody, a purified antibody, or a recombinant antibody, and wherein the antigen-binding fragment is a Fab, Fab′, F(ab′)2, Fv or scFv.

6. The isolated antibody or antigen-binding fragment thereof according to claim 1, wherein the antibody specifically binds an epitope in the aminopeptidase N protein, and wherein said epitope comprises at least one amino acid sequence selected from the group consisting of SEQ ID NO: 52, 53, 54, 55, 56, or 57 or a sequence having at least 92% identity thereto.

7. A chimeric molecule comprising the antibody or antigen-binding fragment thereof according to claim 1, and a compound selected from the group consisting of an antigen, a therapeutic agent, a toxin, a diagnostic marker, a diagnostic label, an imaging agent, and a nanoparticle.

8. The antibody or antigen-binding fragment thereof according to claim 1, wherein said antibody or antigen-binding fragment thereof binds aminopeptidase N with a binding affinity between about 10⁻¹² to 10⁻⁸ M.

9. An isolated nucleic acid encoding the antibody or antigen-binding fragment thereof according to claim 1, wherein said nucleic acid comprises a nucleic acid sequence having at least 95% identity to an HCVR sequence as set forth in SEQ ID NO: 22, and optionally comprises a nucleic acid sequence having at least 95% identity to an LCVR sequence as set forth in SEQ ID NO: 32.

10. A vector comprising the isolated nucleic acid of claim 9.

11. A host cell expressing the antibody or antigen-binding fragment thereof according to claim 1.

12. A pharmaceutical composition comprising the antibody or antigen-binding fragment thereof according to claim 1; a pharmaceutically acceptable diluent or carrier; and, optionally, a further therapeutic agent.

13. (canceled)

14. A method of vaccinating a subject against a disease, the method comprising orally administering to the subject the antibody or antigen-binding fragment thereof according to claim 1.

15. A method for making the antibody or antigen-binding fragment thereof according to claim 1, comprising: (a) introducing into a host cell one or more polynucleotides encoding said antibody or antigen-binding fragment thereof; (b) culturing the host cell under conditions favorable to expression of the one or more polynucleotides; and (c) optionally, isolating the antibody or antigen-binding fragment thereof from the host cell and/or a medium in which the host cell is grown.

16. The method according to claim 14, wherein the vaccination induces an intestinal mucosal immune response.

17. The method according to claim 14, wherein the disease is an intestinal disease.

18. The method according to claim 14, wherein the intestinal disease is selected from the group consisting of inflammatory bowel disease, Crohn's disease, ulcerative colitis, autoimmune disease, and infection.

19. The method according to claim 18, wherein the intestinal disease is an infection caused by a pathogen selected from the group consisting of Escherichia coli, Vibrio, Camylobacter, Clostridium, Salmonella, Yersinia, Lawsonia, Rotavirus, Shigella, PEDV, Cryptosporidium, and Isospora.

20. A method of treating a gut or intestinal infection or inflammation in a subject in need thereof, the method comprising administering to the subject the antibody or antigen-binding fragment thereof according to claim 1.

21. The isolated antibody or antigen-binding fragment thereof according to claim 2, comprising a heavy chain sequence as set forth in SEQ ID: 1 and a light chain sequence as set forth in SEQ ID NO: 11.
